(19) United States
(12) Patent Application Publication
(10) Pub. No.: US 2022/0047711 A1
(43) Pub. Date: Feb. 17, 2022

(54) PHOTOCROSSLINKING PEPTIDES FOR SITE SPECIFIC CONJUGATION TO FC-CONTAINING PROTEINS

(71) Applicant: Genentech, Inc., South San Francisco, CA (US)

(72) Inventors: Jack SADOWSKY, Dublin, CA (US); Neelie Tyana ZACHARIAS, Millbrae, CA (US)

(73) Assignee: Genentech, Inc., South San Francisco, CA (US)

(21) Appl. No.: 17/341,606

(22) Filed: Jun. 8, 2021

Related U.S. Application Data
(63) Continuation of application No. PCT/US2019/064858, filed on Dec. 6, 2019.
(60) Provisional application No. 62/777,375, filed on Dec. 10, 2018.

(51) Int. Cl. A61K 47/68; A61K 45/06; C07K 7/08; A61K 51/10

(52) U.S. Cl. CPC A61K 47/6803; A61K 47/6851; A61K 47/6889; C07K 2319/30; C07K 7/08; A61K 51/1045; A61K 45/06

(57) ABSTRACT

Provided herein are peptides having a photocrosslinking moiety useful for the synthesis of antibody-drug conjugates as well as methods of making and using such conjugates.

### CROSS-REFERENCE TO RELATED APPLICATIONS

This application is a continuation of International Application No. PCT/US2019/064858 having an international filing date of Dec. 6, 2019, which claims benefit under 35 U.S.C. § 119 to U.S. Provisional Patent Application No. 62/777,375 filed Dec. 10, 2018, the entire contents of each of which are incorporated herein by reference and for all purposes.

### SEQUENCE LISTING

This non-provisional patent application incorporates by reference a Sequence Listing submitted with this application as text file entitled P34297US1_SeqList.txt created on Jun. 2, 2021 and having a size of 14,843 kilobytes.

### FIELD OF THE INVENTION

This invention is related to methods of preparing antibody-drug conjugates for therapeutic applications.

### BACKGROUND OF THE INVENTION

Antibody-drug conjugates are an emerging class of targeted prodrug therapeutic agents, with demonstrated in vivo and clinical activity against hyperproliferative disease including cancer, and other indications. (Lambert, J. M.; Berkenblit, A., Antibody-Drug Conjugates for Cancer Treatment. Annual review of medicine 2018, 69, 191-207: Lehar, S. M.; et al., Novel antibody—antibiotic conjugate eliminates intracellular S. aureus. Nature 2015, 527, 323-328: artin, C.; Kizlik-Masson, C.; Pèlegrin, A.; Watier, H.; Viaud-Massuard, M.-C.; Joubert, N., Antibody-drug conjugates: Design and development for therapy and imaging in and beyond cancer, LabEx MAbImprove industrial workshop, Jul. 27-28, 2017, Tours, France. mAbs 2018, 0 (0), 1-12). With the approval of brentuximab vedotin (ADCETRIS®, Seattle Genetics) and ado-trastuzumab emtansine (KADCYLA®, Genentech), the therapeutic potential of antibody drug conjugates (ADCs) providing targeted delivery of pharmaceutically active drug or toxin molecules to specific sites of action has been confirmed, and further research and development has resulted. ADCs are generally composed of an antibody, a pharmaceutically active small molecule drug or toxin (often referred to as the “drug moiety” or “payload”), and an optional linker to connect the two. This protein construct thus joins the small-molecule, a highly potent drug, to the large-molecule antibody, which is selected or engineered to target antigens on a specific cell type, typically a cancer cell. ADCs thus employ the powerful targeting ability of monoclonal antibodies to specifically deliver highly potent, conjugated small molecule therapeutics to a cancer cell (Polakis P. (2005) Current Opinion in Pharmacology 5:382-387).

Successful antibody-drug conjugate development for a given target antigen requires optimization of antibody selection, linker stability, cytotoxic drug potency, and attachment site and mode of linker-drug conjugation to the antibody. (Beck, A.; Goetsch, L.; Dumontet, C.; Corvaia, N., Strategies and challenges for the next generation of antibody—drug conjugates. Nature reviews. Drug discovery 2017, 16 (5), 315-337). More particularly, selective antibody-drug conjugates are characterized by at least one or more of the following: (i) an antibody-drug conjugate formation method wherein the antibody retains sufficient specificity to target antigens and wherein the drug efficacy is maintained; (ii) antibody-drug conjugate stability sufficient to limit drug release in the blood and concomitant damage to non-targeted cells; (iii) sufficient cell membrane transport efficiency (endocytosis) to achieve a therapeutic intracellular antibody-drug conjugate concentration; (iv) sufficient intracellular drug release from the antibody-drug conjugate sufficient to achieve a therapeutic drug concentration; and (v) drug cytotoxicity in nanomolar or sub-nanomolar amounts.

Modification of antibodies with drug moieties (“payloads”) at specific amino acids on the antibody is one goal in the design of effective ADCs. Conjugation of payloads is often to various endogenous amino acids (e.g., lysines or cysteines) present in a wild-type (non-mutated) antibody using chemistry that targets these residues non-specifically (e.g., NHS or other activated esters, maleimides, etc). Such conjugation generates a heterogeneous mixture of products, which in-turn complicates analytical methods required to evaluate and monitor purity, stability, pharmacokinetics and overall in vivo performance of ADCs. By contrast, conjugation strategies that enable site-specific attachment of payloads to specific residues on an antibody enable the generation of more homogeneous products that, in addition to being simpler to analyze, may also display improved safety, stability and pharmacokinetics relative to heterogeneous ADCs (Junutula, J. R. (2008) Nature Biotechnology, 26(8): 925-932).

Site-specific conjugation to antibodies requires the presence of an amino acid residue in the antibody that, among all other amino acids, can be uniquely reacted with chemical functionality on the payload. For such an ADC to have significant in vivo efficacy, the linkage must: (1) not interfere with antigen binding, (2) be stable in circulation and (3) enable release of the payload when the ADC is internalized and degraded in the target cell or tissue. Methods of location-specific derivatization of antibodies have been reported, but most require recombinant engineering of the antibody sequence for introduction of a residue or residues that can be uniquely functionalized with a drug payload to generate a homogeneous ADC (Agarwal, P.; Bertozzi, C. R., Site-specific antibody-drug conjugates: the nexus of bioorthogonal chemistry, protein engineering, and drug development. Bioconjug Chem 2015, 26 (2), 176-92.). In several reported cases where recombinant engineering is not required for site-specific modification, chemical or enzymatic modification of endogenous glycans or disruption of the antibody interchain disulfide bonds is required (For example: Lee, M. T. W.; Maruani, A.; Richards, D. A.; Baker, J. R.; Caddick, S.; Chudasama, V., Enabling the controlled assembly of antibody conjugates with a loading of two modules without antibody engineering. Chem Sci 2017, 8 (3), 2056-2060; van Geel, R.; Wijdeven, M. A.; Heesbeen, R.; Verkade, J. M.; Wasiel, A. A.; van Berkel, S. S.; van Delft, F. L., Chemoenzymatic Conjugation of Toxic Payloads to the Globally Conserved N-Glycan of Native mAbs Provides Homogeneous and Highly Efficacious Antibody-Drug Conjugates. Bioconjug Chem 2015, 26 (11), 2233-42.). These methods introduce one or more steps in, and thereby complicate, the conjugation process and may also affect adversely the biological activity of the final ADC.

Direct methods to prepare antibody-drug conjugates from wild-type antibodies that do not require engineering or modification of the antibody are needed.

### SUMMARY OF THE INVENTION

Provided herein are solutions to these and other problems in the art.

In one embodiment, is a BPA peptide composition comprising a peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11.

In another embodiment is a PhL peptide composition comprising a peptide comprising SEQ ID NO:12, SEQ ID NO:13, SEQ ID NO:14, SEQ ID NO:14, SEQ ID NO:15, SEQ ID NO:16, SEQ ID NO:17, SEQ ID NO:18, or SEQ ID NO:19, SEQ ID NO:20.

In another embodiment is a Tdf peptide composition comprising a peptide comprising SEQ ID NO:21, SEQ ID NO:22, SEQ ID NO:23, SEQ ID NO:24, SEQ ID NO:25, SEQ ID NO:26, SEQ ID NO:27, SEQ ID NO:28, or SEQ ID NO:29.

In still another embodiment is an antibody-drug conjugate comprising an antibody described herein and a BPA peptide described herein covalently attached in the Fc portion of the antibody.

In another embodiment is a method of treating lung cancer, bladder cancer, renal cell cancer (RCC), melanoma, or breast cancer by administering to such a patient an effective amount of an antibody-drug conjugate described herein.

In another embodiment is a method of treating breast cancer, the method comprising administering to a patient having such a breast cancer an effective amount of an antibody-drug conjugate described herein.

In another embodiment is a method of treating lung cancer, the method comprising administering to a patient having such a lung cancer an effective amount of an antibody-drug conjugate described herein.

In another embodiment is a method of treating bladder cancer, the method comprising administering to a patient having such a bladder cancer an effective amount of an antibody-drug conjugate described herein.

In another embodiment is a method of treating kidney cancer, the method comprising administering to a patient having such a kidney cancer an effective amount of an antibody-drug conjugate described herein.

In yet another embodiment is a method of imaging a patient for a tumor, by administering to the patient a composition comprising an ADC described herein and detecting the quantity and location of the label attached to said ADC.

In another embodiment provided herein is a pharmaceutical composition comprising an antibody-drug conjugate composition described herein and a pharmaceutically acceptable excipient

In one embodiment is a method to prepare an antibody-drug conjugate composition described herein by: (i) reacting an antibody under photo-crosslinking conditions with a BPA peptide described herein; (ii) optionally removing a protecting group on the terminal end of the BPA peptide and (iii) reacting the antibody conjugate with a drug (D) as described herein that further comprises a linker to form the antibody-drug conjugate composition having Formula (I), wherein the linker comprises formula (IV) as described herein.

In another embodiment is a method to prepare an antibody-drug conjugate composition as described herein by reacting an antibody described herein under photo-crosslinking conditions with a BPA peptide described herein, wherein the BPA peptide is covalently attached to a drug moiety (D) as described herein through a linker comprising formula (IV) as described herein thereby forming an ADC.

### DESCRIPTION OF THE DRAWINGS

FIG. 1: shows previously reported crystal structure (PDB: 1DN2) of Fc-III peptide bound to human Fc domain.

FIG. 2A and FIG. 2B show photoconjugation of BPA7 described herein to TMab. Conjugated antibody samples were treated with IdeS to create an Fc/2 fragment (FIG. 2A) and a Fab′2 fragment (FIG. 2B). DAR and Fab′2 peak width at half-height (normalized to that of non-irradiated TMab) were monitored throughout optimization. Top row shows Fc/2 and Fab′2 for non-irradiated TMab. Rows A-E show these fragments after photoconjugation to BPA7 to 48 μM (7.2 mg/mL) TMab under various conditions, as follows: Row A shows treatment with 267 μM BPA7, PBS, room temperature, 4 hours; Row B shows treatment with 267 μM BPA7, PBS, on ice, 4 hours; Row C shows treatment with 267 μM BPA7, histidine-acetate, pH 5.5, on ice, 4 hours; Row D shows treatment with 267 μM BPA7, PBS, 267 μM 5-hydroxyindole, on ice, 4 hours; Row E shows treatment with 480 μM BPA7, histidine-acetate, pH 5.5, 267 μM 5-hydroxyindole, 6 hours, on ice.

FIG. 3A shows a full SPR sensorgram for binding of Fc-III. FIG. 3B shows a full SPR sensorgram for binding of BPA7. Raw data are shown in black and curves fit with a one-site binding model. FIG. 3C shows the microscopic rate constants from curve-fitting of sensorgrams for all peptides BPA1-BPA10, including association (kₐ) and dissociation (k_{d}) rates, equilibrium binding dissociation constant (K_{D}), and DAR.

FIG. 4A shows a crystal structure at 2.6 Å resolution of BPA7 conjugated to the Fc region of human IgG1 (PDB ID: 6N9T). Polder Fₒ-F_{c} omit map (grey mesh) is contoured at 4.0 σ r.m.s. within 5 Å of Met-252 and the unnatural Bpa residue on chain A. FIG. 4B shows an overlay of the previously-reported structure of the Fc-bound Fc-III peptide (green, 1DN2) and BPA7 (cyan, 6N9T) shown in sticks. The binding pose of the peptide is well maintained despite the Val-10→ Bpa substitution (RMSD <0.3 Å). FIG. 4C shows an overlay of the BPA7/Fc and Fc-III/Fc complexes highlighting the movement of Met-428 in the Fc necessary to accommodate the terminal aromatic ring of the Bpa residue (arrow).

FIG. 5A shows the synthesis scheme for generation of Tmab conjugated to SATA-BPA7 (top) and SATA-PEG-BPA7 (bottom) crosslinkers with thiols protected by acetylation. FIG. 5B shows mass spectra for the Fc/2 fragment (generated by IdeS) of the starting TMab antibody, Intermediate I, Intermediate II and the final TMab-SATA-PEG-7a-MMAE ADC. Insets indicate efficient removal of the S-acetyl groups (−42 Da) from Intermediate I to give Intermediate II. FIG. 5C shows a size-exclusion chromatogram of Tmab/SATA-PEG-7a/MMAE conjugate with indicated percentage of monomer.

FIG. 6 shows cytotoxicity of TMab/SATA-PEG-BPA7/MMAE photoconjugate (red) and standard THIOMAB™ antibody-drug conjugate against two cell lines, with FIG. 6A showing Sk-BR-3 and FIG. 6B showing KPL-4, expressing high levels of Her2. The IC50 values in Sk-BR-3 cells were 1.7 and 2.0 ng/mL for the photoconjugate and TDC, respectively. The IC50 values in KPL-4 cells were 2.0 and 2.3 ng/mL for the photoconjugate and TDC, respectively.

FIG. 7 shows the stability of TMab/SATA-PEG-BPA7/MMAE conjugate in plasma from various species indicated, as monitored by affinity-capture LC-MS.

FIG. 8 shows FcRn binding to Tmab is inhibited by the presence of increasing amount of Fc-III. Different peptide concentrations were mixed with 1 μM FcRn in a buffer at pH 6.0 and injected on a sensor chip with captured Tmab. For each experiment, the system reached steady-state within 6 minutes and the response (in resonance units (RU)) was measured. A dose-response curve was measured by nonlinear fit to calculate an IC50 of 75±7 nM (dotted line is extrapolation to 0 M Fc-III concentration).

FIG. 9 shows the structure-based sequence alignment of IgGs from human (hu), rabbit (oc), mouse (mu) and rat (rn). Strictly conserved residues are colored red, while semi-conserved residues are colored yellow. Amino acid numbering and secondary structural elements are derived from hulgG1, with Met252 marked with a red star. Sequence alignment was performed with Chimera (v. 1.12).

FIG. 10 shows the comparison of photocrosslinking efficiency of Bpa peptides described herein (BPA1-BPA10) identified as peptides 1a-9a and 10, Photo-Leu peptides described herein (PhL1-PhL9) identified as peptides 1b-9b, and Tdf peptides described herein (Tdf1-Tdf9) identified at peptides 1c-9c to Trastuzumab using the following photocrosslinking conditions: 4 hours UV treatment on ice, pH=5.5 in his-acetate buffer, at 48:480 μM Trastuzumab:peptide final concentrations. Conjugation efficiency is reported as DAR.

FIG. 11A shows chromatograms showing total ion chromatogram (top) and UV signal at 280 nm (bottom). FIG. 11B shows mass spectrum corresponding to major peak indicating singly-charged (M+1) and doubly-charged (M+2) ions corresponding to desired product.

FIG. 12 shows DAR plotted as a function of UV-exposure (hours) at different concentrations of BPA7 ranging from 120 to 960 μM (2.5 to 20-fold molar excess) of BPA7 with Trastuzumab (48 μM). Reactions were performed in 20 mM histidine-acetate, pH 5.5 in the presence of 267 uM 5-hydroxyindole.

FIG. 13 shows FIG. 13A showing a plot of the dissociation constant (K_{d}) as measured by SPR versus the solvent accessible surface area (SASA) for each Bpa substituted peptide (where 1a-9a and 10 correspond to BPA1-BPA9 and BPA10, respectively). SASA for each residue was calculated using Pymol (1.8.6.2) using PDB ID: 1DN2. FIG. 13B shows a plot of K_{d} versus DAR for each peptide in the Bpa series plus the double-cyclic peptide 10.

FIG. 14 shows extracted ion chromatograms for tryptic peptides encompassing Met-252 (DTLMISR) and Met-428 (WQQGNVFSCSVMHEALHNHYTQK, SEQ ID NO:30) for control (unconjugated) Tmab and Tmab conjugated to BPA7. Intensity of peak for Met-252 peptide decreases significantly more relative to that for Met-428 peptide

FIG. 15 shows photoconjugation of BPA7 to Trastuzumab after incubation of the antibody alone or with 5% AAPH (w/v) in the absence or in the presence of free methionine at 37° C. for the indicated timepoints. Values in parentheses indicate % of tryptic peptide containing Met-252 present in the oxidized state as determined by LC/MS-MS analysis.

FIG. 16A shows SEC analysis of Trastuzumab control; FIG. 16B shows SEC analysis of Trastuzumab conjugated to peptide BPA7; FIG. 16C shows SEC analysis of Trastuzumab conjugated to SATA-BPA7, and FIG. 16D shows SEC analysis of Trastuzumab conjugated to SATA-PEG-BPA7.

### DETAILED DESCRIPTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

Unless defined otherwise, technical and scientific terms used herein have the same meaning as commonly understood by one of ordinary skill in the art to which this invention belongs, and are consistent with: Singleton et al. (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

The term “antibody” herein is used in the broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al. (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

An “isolated” antibody is one that has been separated from a component of its natural environment.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); multispecific antibodies formed from antibody fragments, and other fragments (Hudson et al. Nat. Med. 9:129-134 (2003; Pluckthün, in The Pharmacology of Monoclonal Antibodies, vol. 113, Rosenburg and Moore eds., (Springer-Verlag, New York), pp. 269-315 (1994); WO 93/16185; U.S. Pat. Nos. 5,571,894; 5,587,458; 5,869,046. Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See for example, Kindt et al. Kuby Immunology, 6th ed., W.H. Freeman and Co., page 91 (2007).

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition (Chothia and Lesk, (1987) J. Mol. Biol. 196:901-917; Kabat et al., Sequences of Proteins of Immunological Interest, 5th Ed. Public Health Service, National Institutes of Health, Bethesda, Md. (1991). With the exception of CDR1 in VH, CDRs generally comprise the amino acid residues that form the hypervariable loops.

The term “chimeric” antibody refers to an antibody in which a portion of the heavy and/or light chain is derived from a particular source or species, while the remainder of the heavy and/or light chain is derived from a different source or species

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc region” herein is used to define a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to constant domain residues other than hypervariable region (HVR) residues. The FR of a constant domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art. Human antibodies are described generally in van Dijk and van de Winkel, (2001) Curr. Opin. Pharmacol. 5: 368-74; Lonberg, Curr. Opin. Immunol. 20:450-459 (2008).

A “human consensus framework” is a framework region of an antibody which represents the most commonly occurring amino acid residues in a selection of human immunoglobulin VL or VH framework sequences. Generally, the selection of human immunoglobulin VL or VH sequences is from a subgroup of variable domain sequences.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization (Almagro and Fransson, Front. Biosci. 13:1619-1633 (2008); Riechmann et al., Nature 332:323-329 (1988); Queen et al., Proc. Nat'l Acad. Sci. USA 86:10029-10033 (1989); U.S. Pat. Nos. 5,821,337; 7,527,791; 6,982,321; 7,087,409; Kashmiri et al. (2005) Methods 36:25-34; Padlan, (1991) Mol. Immunol. 28:489-498; Dall'Acqua et al. (2005) Methods 36:43-60; Osbourn et al, (2005) Methods 36:61-68; Klimka et al. (2000) Br. J. Cancer 83:252-260).

A “chimeric” antibody comprises a non-human variable region (e.g., a variable region derived from a mouse, rat, hamster, rabbit, or non-human primate, such as a monkey) and a human constant region (U.S. Pat. No. 4,816,567; Morrison et al. (1984) Proc. Natl. Acad. Sci. USA, 81:6851-6855). In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Human framework regions that may be used for humanization include but are not limited to: framework regions selected using the “best-fit” method (Sims et al. J. Immunol. 151:2296 (1993)); framework regions derived from the consensus sequence of human antibodies of a particular subgroup of light or heavy chain variable regions (Carter et al. (1992) Proc. Natl. Acad. Sci. USA, 89:4285; Presta et al. (1993) J. Immunol., 151:2623); human mature (somatically mutated) framework regions or human germline framework regions (Almagro and Fransson, (2008) Front. Biosci. 13:1619-1633); and framework regions derived from screening FR libraries (Baca et al. (1997) J. Biol. Chem. 272:10678-10684; Rosok et al. (1996) J. Biol. Chem. 271:22611-22618).

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and FRs.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Antibodies include fusion proteins comprising an antibody and a protein, drug moiety, label, or some other group. Fusion proteins may be made by recombinant techniques, conjugation, or peptide synthesis, to optimize properties such as pharmacokinetics. The human or humanized antibody of the invention may also be a fusion protein comprising an albumin-binding peptide (ABP) sequence (Dennis et al. (2002) J Biol. Chem. 277:35035-35043; WO 01/45746).

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region (Wright et al. (1997) TIBTECH 15:26-32). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. Such fucosylation variants may have improved ADCC function (US 2003/0157108; US 2004/0093621; Okazaki et al. J. Mol. Biol. 336:1239-1249 (2004); Yamane-Ohnuki et al. (2004) Biotech. Bioeng. 87:614).

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. Antibodies with reduced effector function include those with substitution of one or more of Fc region residues (U.S. Pat. No. 6,737,056). Fc mutants include substitutions at two or more of amino acid positions (U.S. Pat. No. 7,332,581). Antibody variants with improved or diminished binding to FcRs are described. (U.S. Pat. No. 6,737,056; WO 2004/056312; Shields et al. (2001) J. Biol. Chem. 9(2): 6591-6604). An antibody variant may comprise an Fc region with one or more amino acid substitutions which improve ADCC (U.S. Pat. No. 6,194,551, WO 99/51642; Idusogie et al. (2000) J. Immunol. 164: 4178-4184; US2005/0014934).

“Cysteine engineered antibodies” (THIOMAB™), are antibodies in which one or more residues of an antibody are substituted with cysteine residue(s). The substituted residues may occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an antibody-drug conjugate (ADC), also referred to as an immunoconjugate. Examples of a THIOMAB™ include cysteine engineered antibodies in which any one or more of the following residues may be substituted with cysteine: V205 (Kabat numbering) of the light chain; A118 (EU numbering) of the heavy chain; and 5400 (EU numbering) of the heavy chain Fc region, and S121, and K149 of the light chain. Exemplary methods of making cysteine engineered antibodies include, but are not limited to, the methods described, e.g., in U.S. Pat. No. 7,521,541 which is incorporated herein by reference in its entirety and for all purposes.

Thus, the compositions and methods of the invention may be applied to antibody-drug conjugates comprising cysteine engineered antibodies wherein one or more amino acids of a wild-type or parent antibody are replaced with a cysteine amino acid (THIOMAB™). Any form of antibody may be so engineered, i.e. mutated. For example, a parent Fab antibody fragment may be engineered to form a cysteine engineered Fab. Similarly, a parent monoclonal antibody may be engineered to form a THIOMAB™. It should be noted that a single site mutation yields a single engineered cysteine residue in a Fab antibody fragment, while a single site mutation yields two engineered cysteine residues in a full length THIOMAB™, due to the dimeric nature of the IgG antibody. Mutants with replaced (“engineered”) cysteine (Cys) residues are evaluated for the reactivity of the newly introduced, engineered cysteine thiol groups. The thiol reactivity value is a relative, numerical term in the range of 0 to 1.0 and can be measured for any cysteine engineered antibody. Thiol reactivity values of cysteine engineered antibodies of the invention are in the ranges of 0.6 to 1.0; 0.7 to 1.0; or 0.8 to 1.0.

Cysteine amino acids may be engineered at reactive sites in the heavy chain (HC) or light chain (LC) of an antibody and which do not form intrachain or intermolecular disulfide linkages (Junutula, et al., 2008b Nature Biotech., 26(8):925-932; Dornan et al (2009) Blood 114(13):2721-2729; U.S. Pat. Nos. 7,521,541; 7,723,485; WO2009/052249, Shen et al (2012) Nature Biotech., 30(2):184-191; Junutula et al (2008) Jour of Immun. Methods 332:41-52). The engineered cysteine thiols may react with linker reagents or the linker-drug intermediates of the present invention which have thiol-reactive, electrophilic pyridyl disulfide groups to form ADC THIOMAB™ and the drug (D) moiety. The location of the drug moiety can thus be designed, controlled, and known. The drug loading can be controlled since the engineered cysteine thiol groups typically react with thiol-reactive linker reagents or linker-drug intermediates in high yield. Engineering an antibody to introduce a cysteine amino acid by substitution at a single site on the heavy or light chain gives two new cysteines on the symmetrical antibody. A drug loading near 2 can be achieved and near homogeneity of the conjugation product ADC.

Cysteine engineered antibodies preferably retain the antigen binding capability of their wild type, parent antibody counterparts. Thus, cysteine engineered antibodies are capable of binding, preferably specifically, to antigens. Such antigens include, for example, tumor-associated antigens (TAA), cell surface receptor proteins and other cell surface molecules, transmembrane proteins, signaling proteins, cell survival regulatory factors, cell proliferation regulatory factors, molecules associated with (for e.g., known or suspected to contribute functionally to) tissue development or differentiation, lymphokines, cytokines, molecules involved in cell cycle regulation, molecules involved in vasculogenesis and molecules associated with (for e.g., known or suspected to contribute functionally to) angiogenesis. The tumor-associated antigen may be a cluster differentiation factor (i.e., a CD protein). An antigen to which a cysteine engineered antibody is capable of binding may be a member of a subset of one of the above-mentioned categories, wherein the other subset(s) of said category comprise other molecules/antigens that have a distinct characteristic (with respect to the antigen of interest).

Cysteine engineered antibodies are prepared for conjugation with linker-drug intermediates by reduction and reoxidation of intrachain disulfide groups.

Cysteine engineered antibodies which may form the antibody-drug conjugates for use in the methods of this disclosure include cysteine engineered antibodies useful in the treatment of cancer including, but not limited to, antibodies against cell surface receptors and tumor-associated antigens (TAA).

“Tumor-associated antigens” (TAA) are known in the art, and can be prepared for use in generating antibodies using methods and information which are well known in the art. In attempts to discover effective cellular targets for cancer diagnosis and therapy, researchers have sought to identify transmembrane or otherwise tumor-associated polypeptides that are specifically expressed on the surface of one or more particular type(s) of cancer cell as compared to on one or more normal non-cancerous cell(s). Often, such tumor-associated polypeptides are more abundantly expressed on the surface of the cancer cells as compared to on the surface of the non-cancerous cells. The identification of such tumor-associated cell surface antigen polypeptides has given rise to the ability to specifically target cancer cells for destruction via antibody-based therapies.

Examples of tumor-associated antigens (TAA) include, but are not limited to antigens, known in the art, and include names, acronyms, alternative names, Genbank accession numbers and primary reference(s), following nucleic acid and protein sequence identification conventions of the National Center for Biotechnology Information (NCBI). Nucleic acid and protein sequences corresponding to exemplary TAA (1)-(53) below are available in public databases such as GenBank. Tumor-associated antigens targeted by antibodies include all amino acid sequence variants and isoforms possessing at least about 70%, 80%, 85%, 90%, or 95% sequence identity relative to the sequences identified in the cited references, or which exhibit substantially the same biological properties or characteristics as a TAA having a sequence found in the cited references. For example, a TAA having a variant sequence generally is able to bind specifically to an antibody that binds specifically to the TAA.

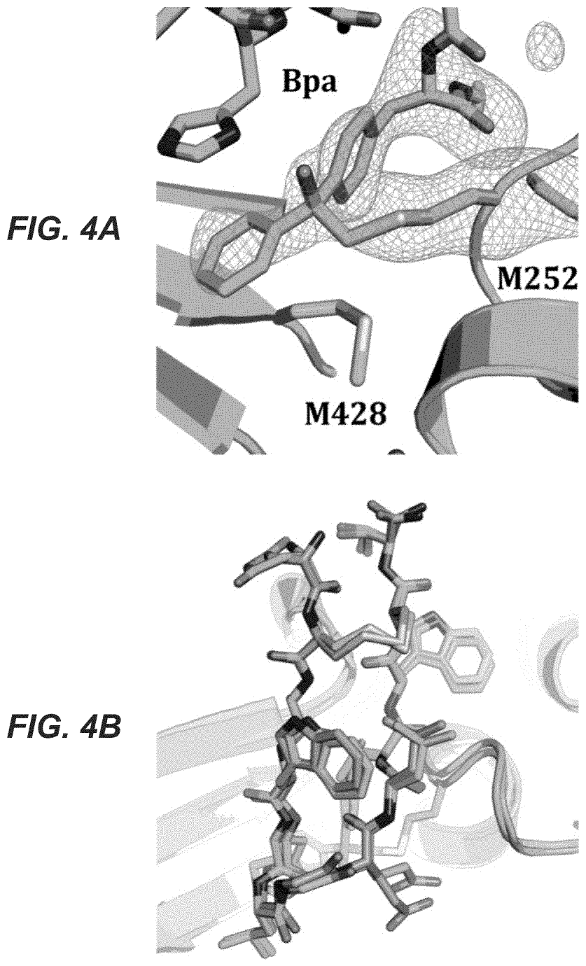

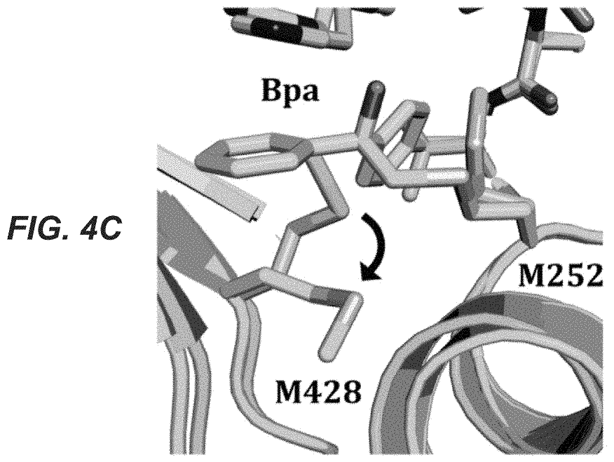

(1) BMPR1B (bone morphogenetic protein receptor-type IB, Genbank accession no. NM_001203) ten Dijke, P., et al. Science 264 (5155):101-104 (1994), Oncogene 14 (11):1377-1382 (1997)); WO2004063362 (claim 2); WO2003042661 (claim 12); US2003134790-A1 (Page 38-39); WO2002102235 (claim 13; Page 296); WO2003055443 (Page 91-92); WO200299122 (Example 2; Page 528-530); WO2003029421 (claim 6); WO2003024392 (claim 2; FIG. 112); WO200298358 (claim 1; Page 183); WO200254940 (Page 100-101); WO200259377(Page 349-350); WO200230268 (claim 27; Page 376); WO200148204 (Example; FIG. 4) NP_001194 bone morphogenetic protein receptor, type IB/pid=NP_001194.1—Cross-references: MIM:603248; NP_001194.1; AY065994.

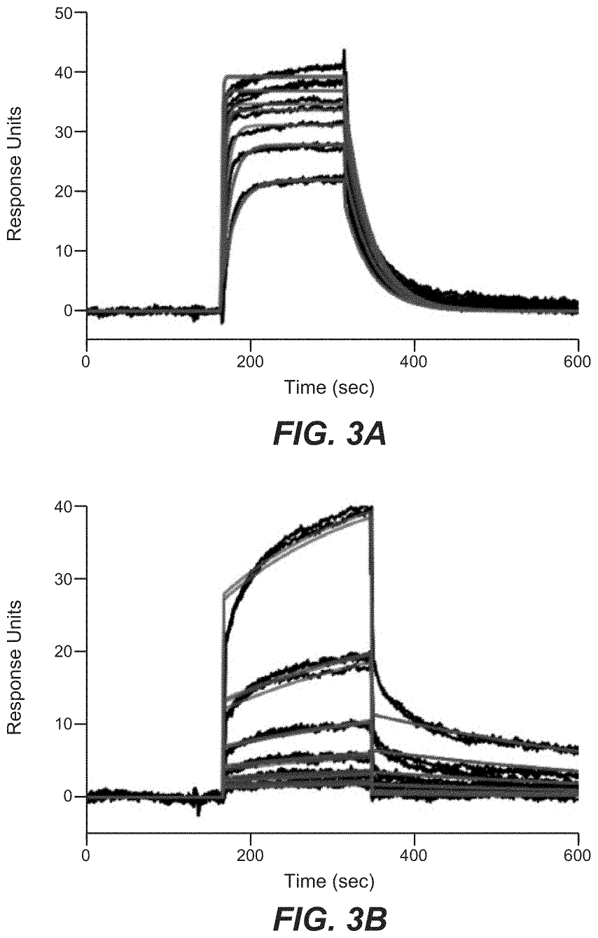

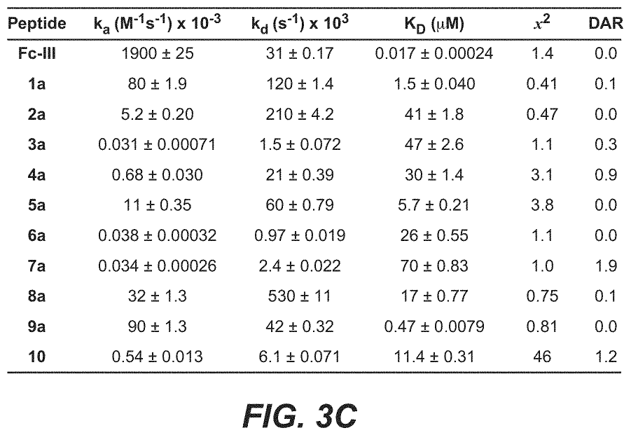

(2) E16 (LAT1, SLC7A5, Genbank accession no. NM_003486) Biochem. Biophys. Res. Commun. 255 (2), 283-288 (1999), Nature 395 (6699):288-291 (1998), Gaugitsch, H. W., et al. (1992) J. Biol. Chem. 267 (16):11267-11273); WO2004048938 (Example 2); WO2004032842 (Example IV); WO2003042661 (claim 12); WO2003016475 (claim 1); WO200278524 (Example 2); WO200299074 (claim 19; Page 127-129); WO200286443 (claim 27; Pages 222, 393); WO2003003906 (claim 10; Page 293); WO200264798 (claim 33; Page 93-95); WO200014228 (claim 5; Page 133-136); US2003224454 (FIG. 3); WO2003025138 (claim 12; Page 150); NP_003477 solute carrier family 7 (cationic amino acid transporter, y+ system), member 5/pid=NP_003477.3—Homo sapiens Cross-references: MIM:600182; NP_003477.3; NM_015923; NM_003486_1.

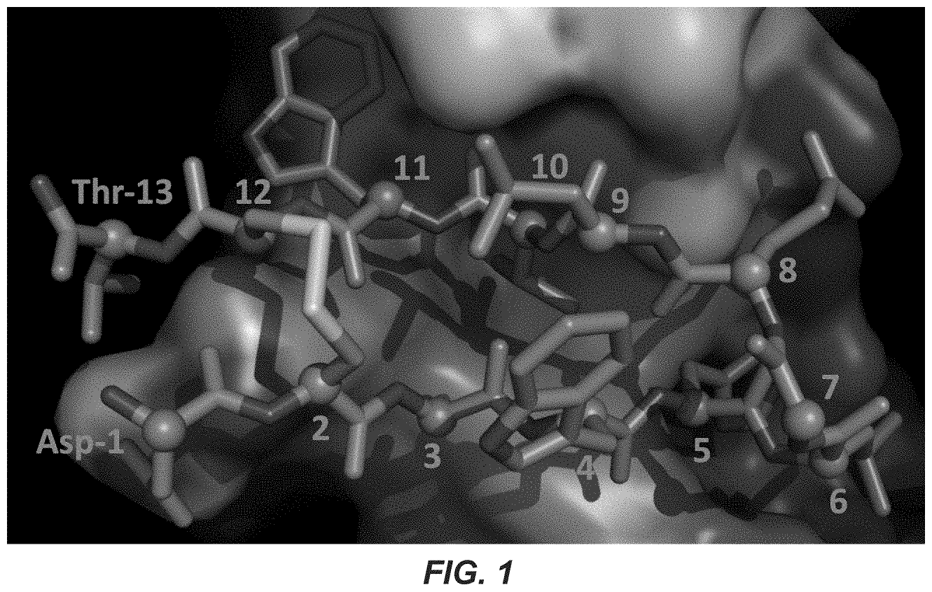

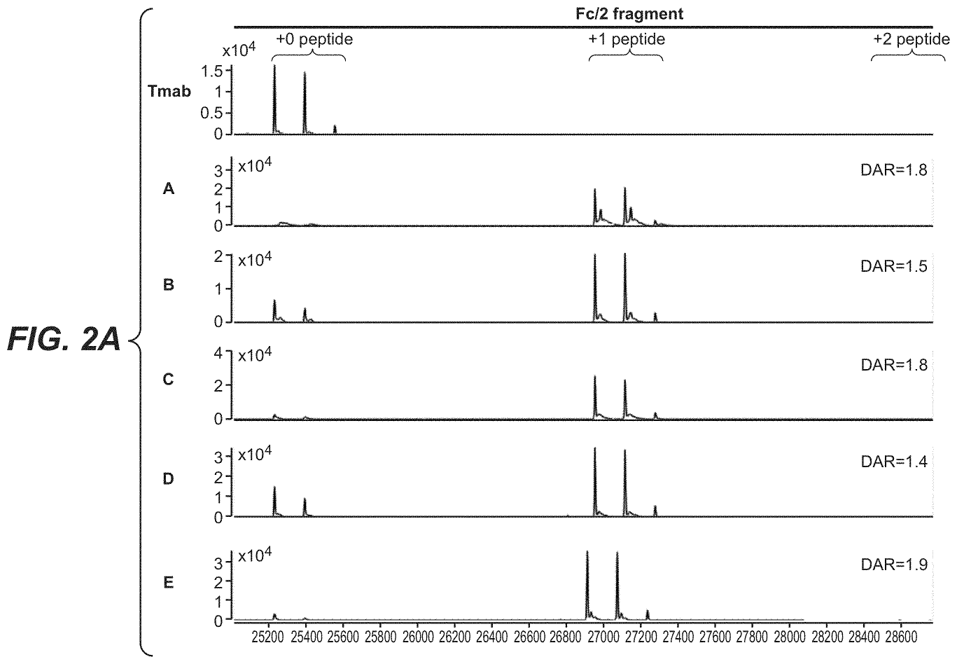

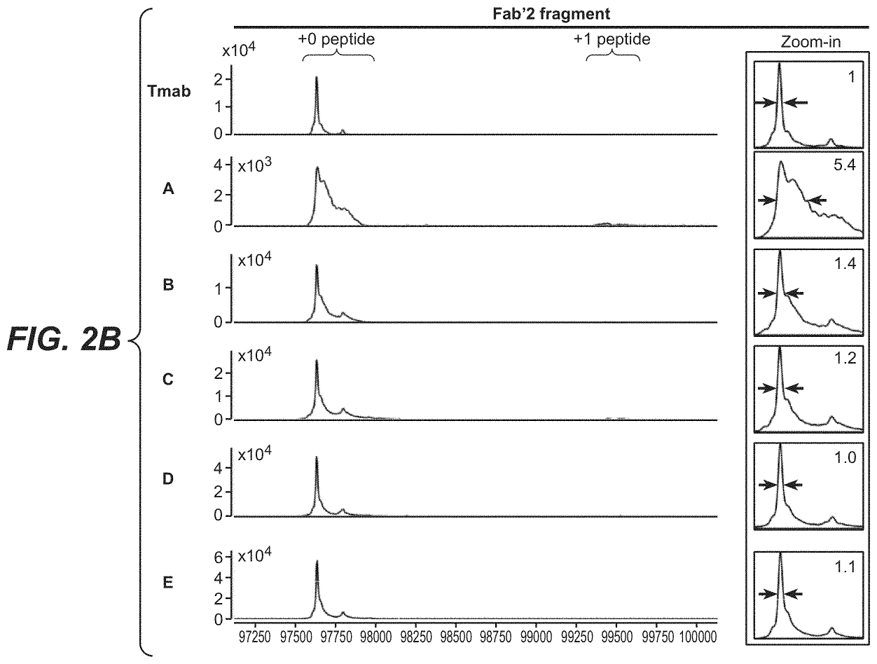

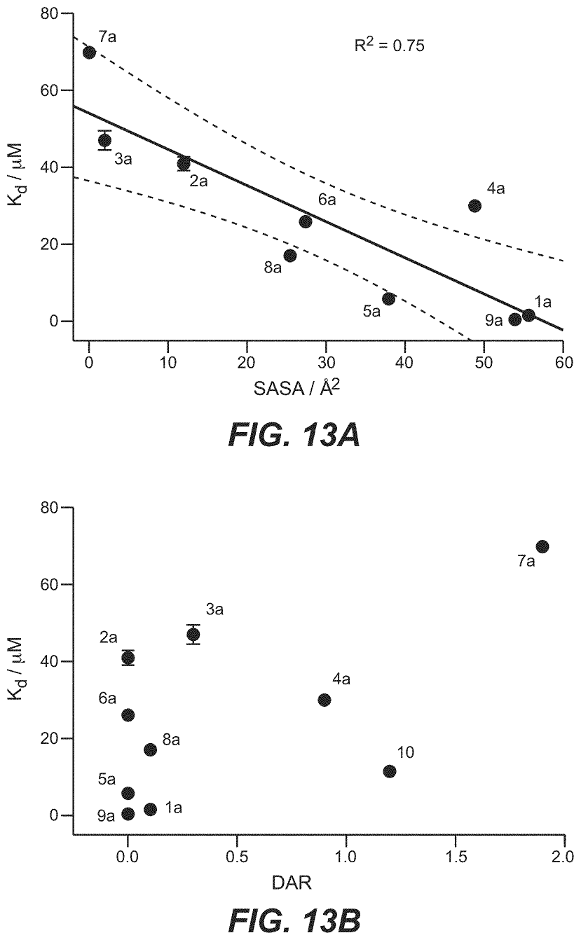

(3) STEAP1 (six transmembrane epithelial antigen of prostate, Genbank accession no. NM_012449) Cancer Res. 61 (15), 5857-5860 (2001), Hubert, R. S., et al. (1999) Proc. Natl. Acad. Sci. U.S.A. 96 (25):14523-14528); WO2004065577 (claim 6); WO2004027049 (FIG. 1L); EP1394274 (Example 11); WO2004016225 (claim 2); WO2003042661 (claim 12); US2003157089 (Example 5); US2003185830 (Example 5); US2003064397 (FIG. 2); WO200289747 (Example 5; Page 618-619); WO2003022995 (Example 9; FIG. 13A, Example 53; Page 173, Example 2; FIG. 2A); NP_036581 six transmembrane epithelial antigen of the prostate Cross-references: MIM:604415; NP_036581.1; NM_012449_1.

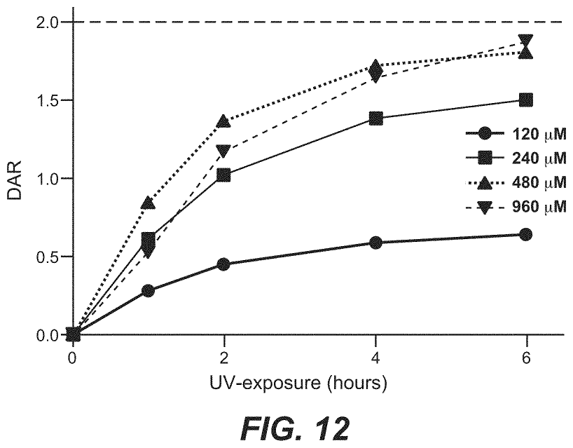

(4) 0772P (CA125, MUC16, Genbank accession no. AF361486) J. Biol. Chem. 276 (29):27371-27375 (2001)); WO2004045553 (claim 14); WO200292836 (claim 6; FIG. 12); WO200283866 (claim 15; Page 116-121); US2003124140 (Example 16); US 798959. Cross-references: GI:34501467; AAK74120.3; AF361486_1.

(5) MPF (MPF, MSLN, SMR, megakaryocyte potentiating factor, mesothelin, Genbank accession no. NM_005823) Yamaguchi, N., et al. Biol. Chem. 269 (2), 805-808 (1994), Proc. Natl. Acad. Sci. U.S.A. 96 (20):11531-11536 (1999), Proc. Natl. Acad. Sci. U.S.A. 93 (1):136-140 (1996), J. Biol. Chem. 270 (37):21984-21990 (1995)); WO2003101283 (claim 14); (WO2002102235 (claim 13; Page 287-288); WO2002101075 (claim 4; Page 308-309); WO200271928 (Page 320-321); WO9410312 (Page 52-57); Cross-references: MIM:601051; NP_005814.2; NM_005823_1.

(6) Napi3b (NAPI-3B, NPTIIb, SLC34A2, solute carrier family 34 (sodium phosphate), member 2, type II sodium-dependent phosphate transporter 3b, Genbank accession no. NM_006424) J. Biol. Chem. 277 (22):19665-19672 (2002), Genomics 62 (2):281-284 (1999), Field, J. A., et al. (1999) Biochem. Biophys. Res. Commun. 258 (3):578-582); WO2004022778 (claim 2); EP1394274 (Example 11); WO2002102235 (claim 13; Page 326); EP875569 (claim 1; Page 17-19); WO200157188 (claim 20; Page 329); WO2004032842 (Example IV); WO200175177 (claim 24; Page 139-140); Cross-references: MIM:604217; NP_006415.1; NM_006424_1.

(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B, Genbank accession no. AB040878) Nagase T., et al. (2000) DNA Res. 7 (2):143-150); WO2004000997 (claim 1); WO2003003984 (claim 1); WO200206339 (claim 1; Page 50); WO200188133 (claim 1; Page 41-43, 48-58); WO2003054152 (claim 20); WO2003101400 (claim 11); Accession: Q9P283; EMBL; AB040878; BAA95969.1. Genew; HGNC:10737.

(8) PSCA hlg (2700050C12Rik, C530008O16Rik, RIKEN cDNA 2700050C12, RIKEN cDNA 2700050C12 gene, Genbank accession no. AY358628); Ross et al. (2002) Cancer Res. 62:2546-2553; US2003129192 (claim 2); US2004044180 (claim 12); US2004044179 (claim 11); US2003096961 (claim 11); US2003232056 (Example 5); WO2003105758 (claim 12); US2003206918 (Example 5); EP1347046 (claim 1); WO2003025148 (claim 20); Cross-references: GI:37182378; AAQ88991.1; AY358628_1.

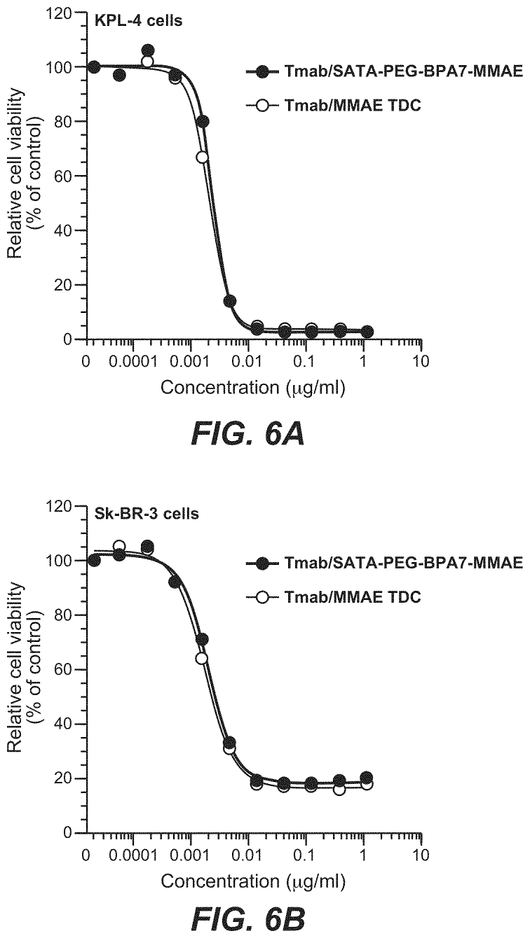

(9) ETBR (Endothelin type B receptor, Genbank accession no. AY275463); Nakamuta M., et al. Biochem. Biophys. Res. Commun. 177, 34-39, 1991; Ogawa Y., et al. Biochem. Biophys. Res. Commun. 178, 248-255, 1991; Arai H., et al. Jpn. Circ. J. 56, 1303-1307, 1992; Arai H., et al. J. Biol. Chem. 268, 3463-3470, 1993; Sakamoto A., Yanagisawa M., et al. Biochem. Biophys. Res. Commun. 178, 656-663, 1991; Elshourbagy N. A., et al. J. Biol. Chem. 268, 3873-3879, 1993; Haendler B., et al. J. Cardiovasc. Pharmacol. 20, s1-S4, 1992; Tsutsumi M., et al. Gene 228, 43-49, 1999; Strausberg R. L., et al. Proc. Natl. Acad. Sci. U.S.A. 99, 16899-16903, 2002; Bourgeois C., et al. J. Clin. Endocrinol. Metab. 82, 3116-3123, 1997; Okamoto Y., et al. Biol. Chem. 272, 21589-21596, 1997; Verheij J. B., et al. Am. J. Med. Genet. 108, 223-225, 2002; Hofstra R. M. W., et al. Eur. J. Hum. Genet. 5, 180-185, 1997; Puffenberger E. G., et al. Cell 79, 1257-1266, 1994; Attie T., et al., Hum. Mol. Genet. 4, 2407-2409, 1995; Auricchio A., et al. Hum. Mol. Genet. 5:351-354, 1996; Amiel J., et al. Hum. Mol. Genet. 5, 355-357, 1996; Hofstra R. M. W., et al. Nat. Genet. 12, 445-447, 1996; Svensson P. J., et al. Hum. Genet. 103, 145-148, 1998; Fuchs S., et al. Mol. Med. 7, 115-124, 2001; Pingault V., et al. (2002) Hum. Genet. 111, 198-206; WO2004045516 (claim 1); WO2004048938 (Example 2); WO2004040000 (claim 151); WO2003087768 (claim 1); WO2003016475 (claim 1); WO2003016475 (claim 1); WO200261087 (FIG. 1); WO2003016494 (FIG. 6); WO2003025138 (claim 12; Page 144); WO200198351 (claim 1; Page 124-125); EP522868 (claim 8; FIG. 2); WO200177172 (claim 1; Page 297-299); US2003109676; U.S. Pat. No. 6,518,404 (FIG. 3); U.S. Pat. No. 5,773,223 (claim 1a; Col 31-34); WO2004001004.

(10) MSG783 (RNF124, hypothetical protein FLJ20315, Genbank accession no. NM_017763); WO2003104275 (claim 1); WO2004046342 (Example 2); WO2003042661 (claim 12); WO2003083074 (claim 14; Page 61); WO2003018621 (claim 1); WO2003024392 (claim 2; FIG. 93); WO200166689 (Example 6); Cross-references: LocusID:54894; NP_060233.2; NM_017763_1.

(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138) Lab. Invest. 82 (11):1573-1582 (2002)); WO2003087306; US2003064397 (claim 1; FIG. 1); WO200272596 (claim 13; Page 54-55); WO200172962 (claim 1; FIG. 4B); WO2003104270 (claim 11); WO2003104270 (claim 16); US2004005598 (claim 22); WO2003042661 (claim 12); US2003060612 (claim 12; FIG. 10); WO200226822 (claim 23; FIG. 2); WO200216429 (claim 12; FIG. 10); Cross-references: GI:22655488; AAN04080.1; AF455138_1.

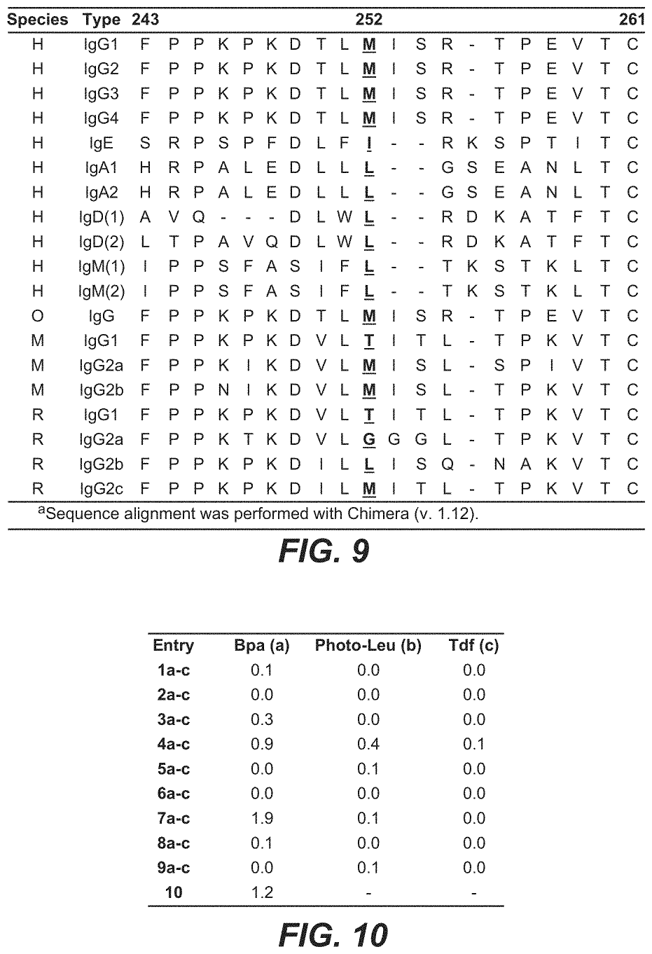

(12) TrpM4 (BR22450, FLJ20041, TRPM4, TRPM4B, transient receptor potential cation channel, subfamily M, member 4, Genbank accession no. NM_017636) Xu, X. Z., et al. Proc. Natl. Acad. Sci. U.S.A. 98 (19):10692-10697 (2001), Cell 109 (3):397-407 (2002), J. Biol. Chem. 278 (33):30813-30820 (2003)); US2003143557 (claim 4); WO200040614 (claim 14; Page 100-103); WO200210382 (claim 1; FIG. 9A); WO2003042661 (claim 12); WO200230268 (claim 27; Page 391); US2003219806 (claim 4); WO200162794 (claim 14; FIG. 1A-D); Cross-references: MIM:606936; NP_060106.2; NM_017636_1.

(13) CRIPTO (CR, CR1, CRGF, CRIPTO, TDGF1, teratocarcinoma-derived growth factor, Genbank accession no. NP_003203 or NM_003212) Ciccodicola, A., et al. EMBO J. 8 (7):1987-1991 (1989), Am. J. Hum. Genet. 49 (3):555-565 (1991)); US2003224411 (claim 1); WO2003083041 (Example 1); WO2003034984 (claim 12); WO200288170 (claim 2; Page 52-53); WO2003024392 (claim 2; FIG. 58); WO200216413 (claim 1; Page 94-95, 105); WO200222808 (claim 2; FIG. 1); U.S. Pat. No. 5,854,399 (Example 2; Col 17-18); U.S. Pat. No. 5,792,616 (FIG. 2); Cross-references: MIM:187395; NP_003203.1; NM_003212_1.

(14) CD21 (CR2 (Complement receptor 2) or C3DR (C3d/Epstein Barr virus receptor) or Hs.73792 Genbank accession no. M26004) Fujisaku et al. (1989) J. Biol. Chem. 264 (4):2118-2125); Weis J. J., et al. J. Exp. Med. 167, 1047-1066, 1988; Moore M., et al. Proc. Natl. Acad. Sci. U.S.A. 84, 9194-9198, 1987; Barel M., et al. Mol. Immunol. 35, 1025-1031, 1998; Weis J. J., et al. Proc. Natl. Acad. Sci. U.S.A. 83, 5639-5643, 1986; Sinha S. K., et al. (1993) J. Immunol. 150, 5311-5320; WO2004045520 (Example 4); US2004005538 (Example 1); WO2003062401 (claim 9); WO2004045520 (Example 4); WO9102536 (FIGS. 9.1-9.9); WO2004020595 (claim 1); Accession: P20023; Q13866; Q14212; EMBL; M26004; AAA35786.1.

(15) CD79b (CD79B, CD79β, IGb (immunoglobulin-associated beta), B29, Genbank accession no. NM_000626 or 11038674) Proc. Natl. Acad. Sci. U.S.A. (2003) 100 (7):4126-4131, Blood (2002) 100 (9):3068-3076, Muller et al. (1992) Eur. J. Immunol. 22 (6):1621-1625); WO2004016225 (claim 2, FIG. 140); WO2003087768, US2004101874 (claim 1, page 102); WO2003062401 (claim 9); WO200278524 (Example 2); US2002150573 (claim 5, page 15); U.S. Pat. No. 5,644,033; WO2003048202 (claim 1, pages 306 and 309); WO 99/558658, U.S. Pat. No. 6,534,482 (claim 13, FIG. 17A/B); WO200055351 (claim 11, pages 1145-1146); Cross-references: MIM:147245; NP_000617.1; NM_000626_1.

(16) FcRH2 (IFGP4, IRTA4, SPAP1A (SH2 domain containing phosphatase anchor protein 1a), SPAP1B, SPAP1C, Genbank accession no. NM_030764, AY358130) Genome Res. 13 (10):2265-2270 (2003), Immunogenetics 54 (2):87-95 (2002), Blood 99 (8):2662-2669 (2002), Proc. Natl. Acad. Sci. U.S.A. 98 (17):9772-9777 (2001), Xu, M. J., et al. (2001) Biochem. Biophys. Res. Commun. 280 (3):768-775; WO2004016225 (claim 2); WO2003077836; WO200138490 (claim 5; FIG. 18D-1-18D-2); WO2003097803 (claim 12); WO2003089624 (claim 25); Cross-references: MIM:606509; NP_110391.2; NM_030764_1.

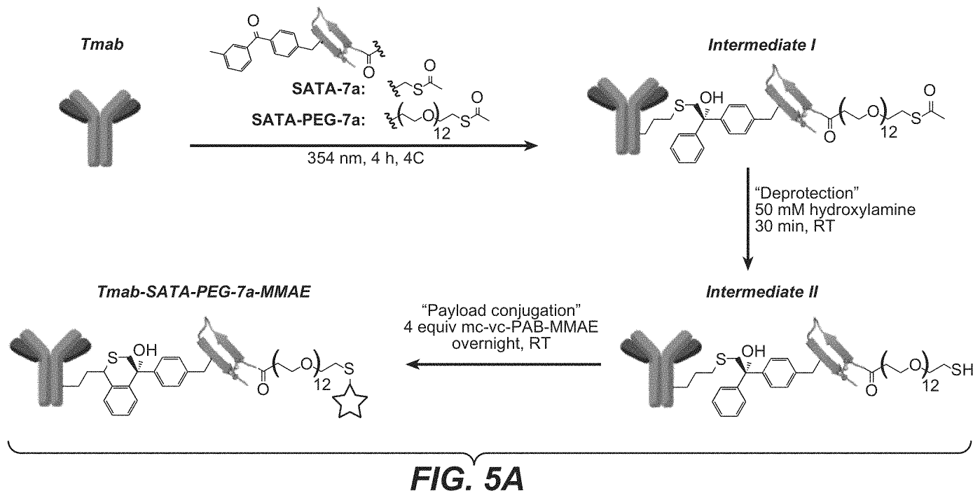

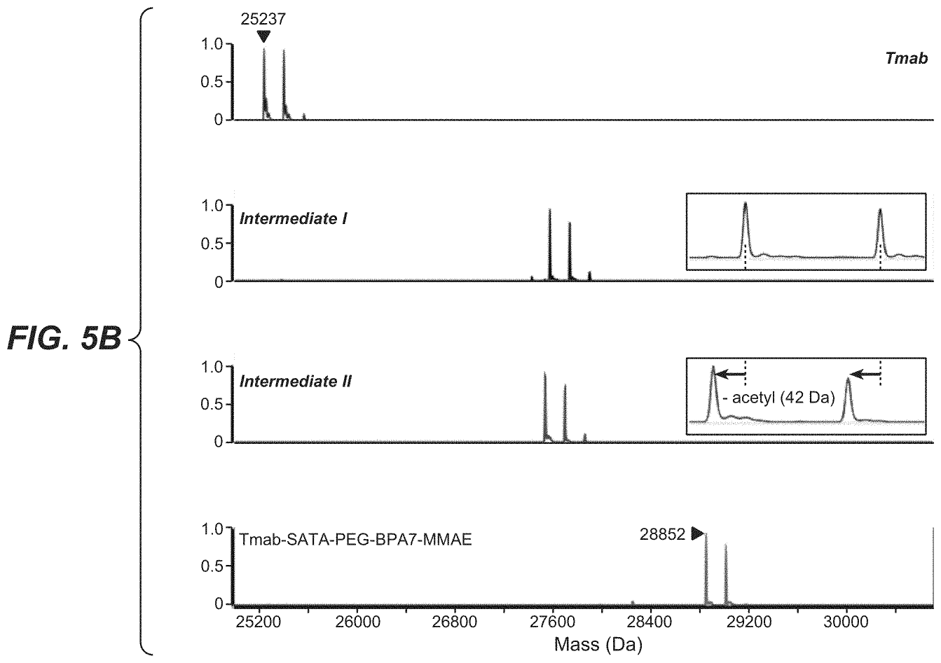

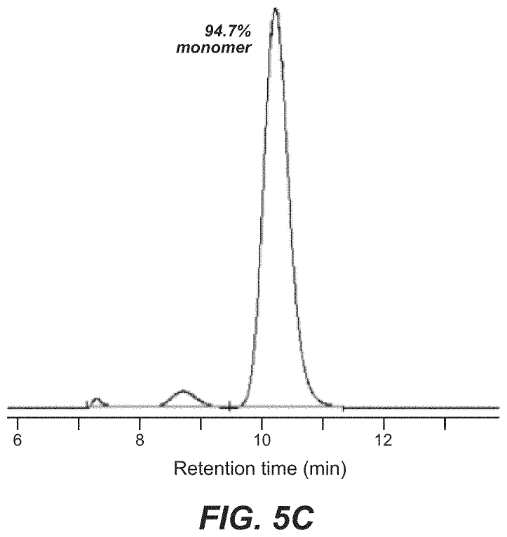

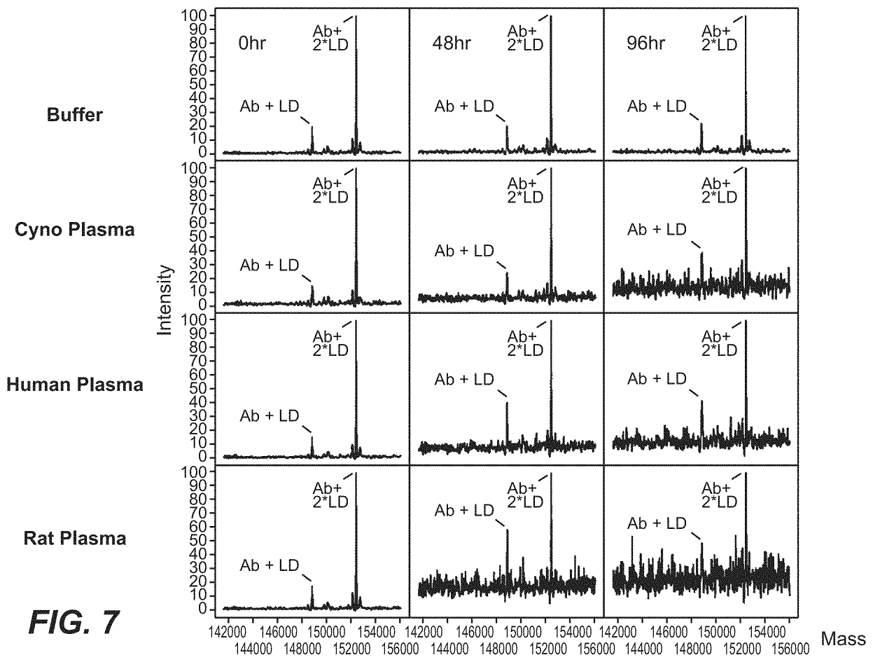

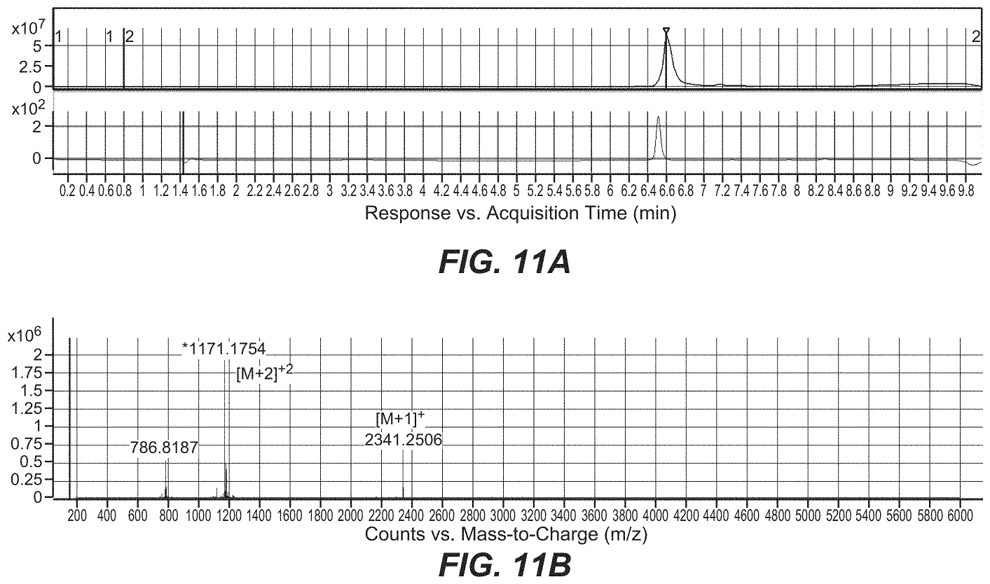

(17) HER2 (ErbB2, Genbank accession no. M11730) Coussens L., et al. Science (1985) 230(4730):1132-1139); Yamamoto T., et al. Nature 319, 230-234, 1986; Semba K., et al. Proc. Natl. Acad. Sci. U.S.A. 82, 6497-6501, 1985; Swiercz J. M., et al. J. Cell Biol. 165, 869-880, 2004; Kuhns J. J., et al. J. Biol. Chem. 274, 36422-36427, 1999; Cho H.-S., et al. Nature 421, 756-760, 2003; Ehsani A., et al. (1993) Genomics 15, 426-429; WO2004048938 (Example 2); WO2004027049 (FIG. 11); WO2004009622; WO2003081210; WO2003089904 (claim 9); WO2003016475 (claim 1); US2003118592; WO2003008537 (claim 1); WO2003055439 (claim 29; FIG. 1A-B); WO2003025228 (claim 37; FIG. 5C); WO200222636 (Example 13; Page 95-107); WO200212341 (claim 68; FIG. 7); WO200213847 (Page 71-74); WO200214503 (Page 114-117); WO200153463 (claim 2; Page 41-46); WO200141787 (Page 15); WO200044899 (claim 52; FIG. 7); WO200020579 (claim 3; FIG. 2); U.S. Pat. No. 5,869,445 (claim 3; Col 31-38); WO9630514 (claim 2; Page 56-61); EP1439393 (claim 7); WO2004043361 (claim 7); WO2004022709; WO200100244 (Example 3; FIG. 4); Accession: P04626; EMBL; M11767; AAA35808.1. EMBL; M11761; AAA35808.1.

(18) NCA (CEACAM6, Genbank accession no. M18728); Barnett T., et al. Genomics 3, 59-66, 1988; Tawaragi Y., et al. Biochem. Biophys. Res. Commun. 150, 89-96, 1988; Strausberg R. L., et al. Proc. Natl. Acad. Sci. U.S.A. 99:16899-16903, 2002; WO2004063709; EP1439393 (claim 7); WO2004044178 (Example 4); WO2004031238; WO2003042661 (claim 12); WO200278524 (Example 2); WO200286443 (claim 27; Page 427); WO200260317 (claim 2); Accession: P40199; Q14920; EMBL; M29541; AAA59915.1. EMBL; M18728.

(19) MDP (DPEP1, Genbank accession no. BC017023) Proc. Natl. Acad. Sci. U.S.A. 99 (26):16899-16903 (2002)); WO2003016475 (claim 1); WO200264798 (claim 33; Page 85-87); JP05003790 (FIG. 6-8); WO9946284 (FIG. 9); Cross-references: MIM:179780; AAH17023.1; BC017023_1.

(20) IL20Rα (IL20Ra, ZCYTOR7, Genbank accession no. AF184971); Clark H. F., et al. Genome Res. 13, 2265-2270, 2003; Mungall A. J., et al. Nature 425, 805-811, 2003; Blumberg H., et al. Cell 104, 9-19, 2001; Dumoutier L., et al. J. Immunol. 167, 3545-3549, 2001; Parrish-Novak J., et al. J. Biol. Chem. 277, 47517-47523, 2002; Pletnev S., et al. (2003) Biochemistry 42:12617-12624; Sheikh F., et al. (2004) J. Immunol. 172, 2006-2010; EP1394274 (Example 11); US2004005320 (Example 5); WO2003029262 (Page 74-75); WO2003002717 (claim 2; Page 63); WO200222153 (Page 45-47); US2002042366 (Page 20-21); WO200146261 (Page 57-59); WO200146232 (Page 63-65); WO9837193 (claim 1; Page 55-59); Accession: Q9UHF4; Q6UWA9; Q96SH8; EMBL; AF184971; AAF01320.1.

(21) Brevican (BCAN, BEHAB, Genbank accession no. AF229053) Gary S. C., et al. Gene 256, 139-147, 2000; Clark H. F., et al. Genome Res. 13, 2265-2270, 2003; Strausberg R. L., et al. Proc. Natl. Acad. Sci. U.S.A. 99, 16899-16903, 2002; US2003186372 (claim 11); US2003186373 (claim 11); US2003119131 (claim 1; FIG. 52); US2003119122 (claim 1; FIG. 52); US2003119126 (claim 1); US2003119121 (claim 1; FIG. 52); US2003119129 (claim 1); US2003119130 (claim 1); US2003119128 (claim 1; FIG. 52); US2003119125 (claim 1); WO2003016475 (claim 1); WO200202634 (claim 1).

(22) EphB2R (DRT, ERK, Hek5, EPHT3, Tyro5, Genbank accession no. NM_004442) Chan, J. and Watt, V. M., Oncogene 6 (6), 1057-1061 (1991) Oncogene 10 (5):897-905 (1995), Annu. Rev. Neurosci. 21:309-345 (1998), Int. Rev. Cytol. 196:177-244 (2000)); WO2003042661 (claim 12); WO200053216 (claim 1; Page 41); WO2004065576 (claim 1); WO2004020583 (claim 9); WO2003004529 (Page 128-132); WO200053216 (claim 1; Page 42); Cross-references: MIM:600997; NP_004433.2; NM_004442_1.

(23) ASLG659 (B7h, Genbank accession no. AX092328) US20040101899 (claim 2); WO2003104399 (claim 11); WO2004000221 (FIG. 3); US2003165504 (claim 1); US2003124140 (Example 2); US2003065143 (FIG. 60); WO2002102235 (claim 13; Page 299); US2003091580 (Example 2); WO200210187 (claim 6; FIG. 10); WO200194641 (claim 12; FIG. 7b); WO200202624 (claim 13; FIG. 1A-1B); US2002034749 (claim 54; Page 45-46); WO200206317 (Example 2; Page 320-321, claim 34; Page 321-322); WO200271928 (Page 468-469); WO200202587 (Example 1; FIG. 1); WO200140269 (Example 3; Pages 190-192); WO200036107 (Example 2; Page 205-207); WO2004053079 (claim 12); WO2003004989 (claim 1); WO200271928 (Page 233-234, 452-453); WO 0116318.

(24) PSCA (Prostate stem cell antigen precursor, Genbank accession no. AJ297436) Reiter R. E., et al. Proc. Natl. Acad. Sci. U.S.A. 95, 1735-1740, 1998; Gu Z., et al. Oncogene 19, 1288-1296, 2000; Biochem. Biophys. Res. Commun. (2000) 275(3):783-788; WO2004022709; EP1394274 (Example 11); US2004018553 (claim 17); WO2003008537 (claim 1); WO200281646 (claim 1; Page 164); WO 2003003906 (claim 10; Page 288); WO 200140309 (Example 1; FIG. 17); US 2001055751 (Example 1; FIG. 1b); WO 200032752 (claim 18; FIG. 1); WO 1998/51805 (claim 17; Page 97); WO 1998/51824 (claim 10; Page 94); WO 1998/40403 (claim 2; FIG. 1B); Accession: 043653; EMBL; AF043498; AAC39607.1.

(25) GEDA (Genbank accession No. AY260763); AAP14954 lipoma HMGIC fusion-partner-like protein/pid=AAP14954.1—Homo sapiens Species: Homo sapiens (human) WO2003054152 (claim 20); WO2003000842 (claim 1); WO2003023013 (Example 3, claim 20); US2003194704 (claim 45); Cross-references: GI:30102449; AAP14954.1; AY260763_1.

(26) BAFF-R (B cell-activating factor receptor, BLyS receptor 3, BR3, Genbank accession No. AF116456); BAFF receptor/pid=NP_443177.1—Homo sapiens Thompson, J. S., et al. Science 293 (5537), 2108-2111 (2001); WO2004058309; WO2004011611; WO2003045422 (Example; Page 32-33); WO2003014294 (claim 35; FIG. 6B); WO2003035846 (claim 70; Page 615-616); WO200294852 (Col 136-137); WO200238766 (claim 3; Page 133); WO200224909 (Example 3; FIG. 3); Cross-references: MIM:606269; NP_443177.1; NM_052945_1; AF132600.

(27) CD22 (B-cell receptor CD22-B isoform, BL-CAM, Lyb-8, Lyb8, SIGLEC-2, FLJ22814, Genbank accession No. AK026467); Wilson et al. (1991) J. Exp. Med. 173:137-146; WO2003072036 (claim 1; FIG. 1); Cross-references: MIM:107266; NP_001762.1; NM_001771_1.

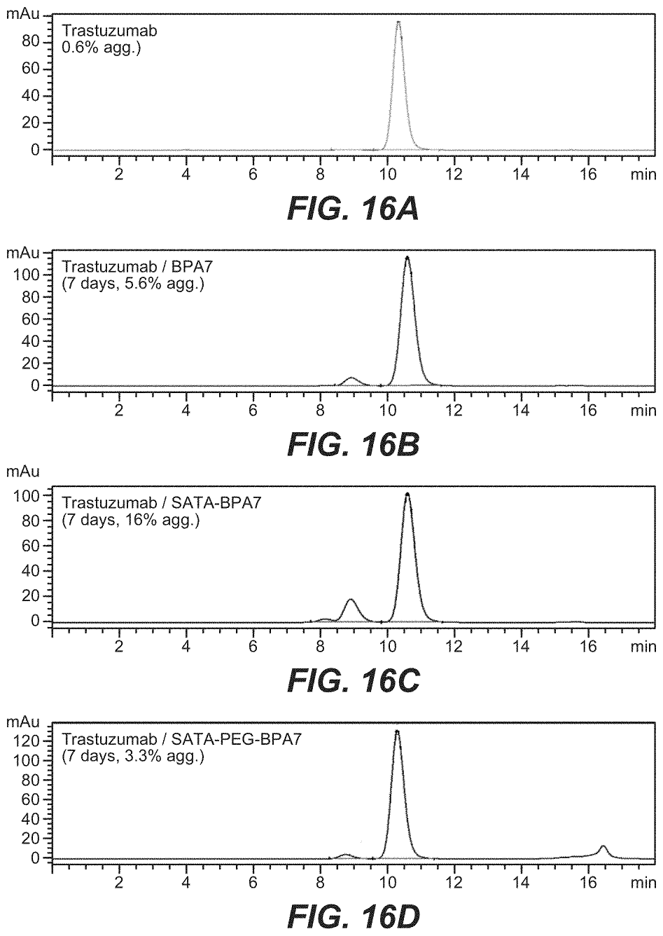

(28) CD79a (CD79A, CD79α, immunoglobulin-associated alpha, a B cell-specific protein that covalently interacts with Ig beta (CD79B) and forms a complex on the surface with Ig M molecules, transduces a signal involved in B-cell differentiation), pl: 4.84, MW: 25028 TM: 2 [P] Gene Chromosome: 19q13.2, Genbank accession No. NP_001774.10) WO2003088808, US20030228319; WO2003062401 (claim 9); US2002150573 (claim 4, pages 13-14); WO9958658 (claim 13, FIG. 16); WO9207574 (FIG. 1); U.S. Pat. No. 5,644,033; Ha et al. (1992) J. Immunol. 148(5):1526-1531; Mueller et al. (1992) Eur. J. Biochem. 22:1621-1625; Hashimoto et al. (1994) Immunogenetics 40(4):287-295; Preud'homme et al. (1992) Clin. Exp. Immunol. 90(1):141-146; Yu et al. (1992) J. Immunol. 148(2) 633-637; Sakaguchi et al. (1988) EMBO J. 7(11):3457-3464.

(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, pl: 8.54 MW: 41959 TM: 7 [P] Gene Chromosome: 11q23.3, Genbank accession No. NP_001707.1) WO 2004040000; WO2004/015426; US2003105292 (Example 2); U.S. Pat. No. 6,555,339 (Example 2); WO 2002/61087 (FIG. 1); WO200157188 (claim 20, page 269); WO200172830 (pages 12-13); WO 2000/22129 (Example 1, pages 152-153, Example 2, pages 254-256); WO 199928468 (claim 1, page 38); U.S. Pat. No. 5,440,021 (Example 2, col 49-52); WO9428931 (pages 56-58); WO 1992/17497 (claim 7, FIG. 5); Dobner et al. (1992) Eur. J. Immunol. 22:2795-2799; Barella et al. (1995) Biochem. J. 309:773-779.

(30) HLA-DOB (Beta subunit of MHC class II molecule (Ia antigen) that binds peptides and presents them to CD4+ T lymphocytes); 273 aa, pl: 6.56 MW: 30820 TM: 1 [P] Gene Chromosome: 6p21.3, Genbank accession No. NP_002111.1) Tonnelle et al. (1985) EMBO J. 4(11):2839-2847; Jonsson et al. (1989) Immunogenetics 29(6):411-413; Beck et al. (1992) J. Mol. Biol. 228:433-441; Strausberg et al. (2002) Proc. Natl. Acad. Sci USA 99:16899-16903; Servenius et al. (1987) J. Biol. Chem. 262:8759-8766; Beck et al. (1996) J. Mol. Biol. 255:1-13; Naruse et al. (2002) Tissue Antigens 59:512-519; WO9958658 (claim 13, FIG. 15); U.S. Pat. No. 6,153,408 (Col 35-38); U.S. Pat. No. 5,976,551 (col 168-170); U.S. Pat. No. 6,011,146 (col 145-146); Kasahara et al. (1989) Immunogenetics 30(1):66-68; Larhammar et al. (1985) J. Biol. Chem. 260(26):14111-14119.

(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pl: 7.63, MW: 47206 TM: 1 [P] Gene Chromosome: 17p13.3, Genbank accession No. NP_002552.2) Le et al. (1997) FEBS Lett. 418(1-2):195-199; WO2004047749; WO2003072035 (claim 10); Touchman et al. (2000) Genome Res. 10:165-173; WO200222660 (claim 20); WO2003093444 (claim 1); WO2003087768 (claim 1); WO2003029277 (page 82).

(32) CD72 (B-cell differentiation antigen CD72, Lyb-2), pl: 8.66, MW: 40225 TM: 1 [P] Gene Chromosome: 9p13.3, Genbank accession No. NP_001773.1) WO2004042346 (claim 65); WO 2003/026493 (pages 51-52, 57-58); WO 2000/75655 (pages 105-106); Von Hoegen et al. (1990) J. Immunol. 144(12):4870-4877; Strausberg et al. (2002) Proc. Natl. Acad. Sci USA 99:16899-16903.

(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosus); 661 aa, pl: 6.20, MW: 74147 TM: 1 [P] Gene Chromosome: 5q12, Genbank accession No. NP_005573.1) US2002193567; WO9707198 (claim 11, pages 39-42); Miura et al. (1996) Genomics 38(3):299-304; Miura et al. (1998) Blood 92:2815-2822; WO2003083047; WO9744452 (claim 8, pages 57-61); WO200012130 (pages 24-26).

(34) FcRH1 (Fc receptor-like protein 1, a putative receptor for the immunoglobulin Fc domain that contains C2 type Ig-like and ITAM domains, may have a role in B-lymphocyte differentiation); 429 aa, pl: 5.28, MW: 46925 TM: 1 [P] Gene Chromosome: 1q21-1q22, Genbank accession No. NP_443170.1) WO2003077836; WO200138490 (claim 6, FIG. 18E-1-18-E-2); Davis et al. (2001) Proc. Natl. Acad. Sci USA 98(17):9772-9777; WO2003089624 (claim 8); EP1347046 (claim 1); WO2003089624 (claim 7).

(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pl: 6.88 MW: 106468 TM: 1 [P] Gene Chromosome: 1q21, Genbank accession No. Human:AF343662, AF343663, AF343664, AF343665, AF369794, AF397453, AK090423, AK090475, AL834187, AY358085; Mouse:AK089756, AY158090, AY506558; NP_112571.1. WO2003024392 (claim 2, FIG. 97); Nakayama et al. (2000) Biochem. Biophys. Res. Commun. 277(1):124-127; WO2003077836; WO200138490 (claim 3, FIG. 18B-1-18B-2).

(36) TENB2 (TMEFF2, tomoregulin, TPEF, HPP1, TR, putative transmembrane proteoglycan, related to the EGF/heregulin family of growth factors and follistatin); 374 aa, NCBI Accession: AAD55776, AAF91397, AAG49451, NCBI RefSeq: NP_057276; NCBI Gene: 23671; OMIM: 605734; SwissProt Q9UIK5; Genbank accession No. AF179274; AY358907, CAF85723, CQ782436 WO 2004074320; JP 2004113151; WO 2003042661; WO2003009814; EP1295944 (pages 69-70); WO 200230268 (page 329); WO 200190304; US2004249130; US 2004022727; WO 2004063355; US 2004197325; US2003232350; US2004005563; US 2003124579; Horie et al. (2000) Genomics 67:146-152; Uchida et al. (1999) Biochem. Biophys. Res. Commun. 266:593-602; Liang et al. (2000) Cancer Res. 60:4907-12; Glynne-Jones et al. (2001) Int J Cancer. October 15; 94(2):178-84.

(37) PMEL17 (silver homolog; SILV; D12S53E; PMEL17; SI; SIL); ME20; gp100) BC001414; BT007202; M32295; M77348; NM_006928; McGlinchey, R. P. et al. (2009) Proc. Natl. Acad. Sci. U.S.A. 106 (33), 13731-13736; Kummer, M. P. et al. (2009) J. Biol. Chem. 284 (4), 2296-2306.

(38) TMEFF1 (transmembrane protein with EGF-like and two follistatin-like domains 1; Tomoregulin-1); H7365; C9orf2; C9ORF2; U19878; X83961; NM_080655; NM_003692; Harms, P. W. (2003) Genes Dev. 17 (21), 2624-2629; Gery, S. et al. (2003) Oncogene 22 (18):2723-2727.

(39) GDNF-Ra1 (GDNF family receptor alpha 1; GFRA1; GDNFR; GDNFRA; RETL1; TRNR1; RET1L; GDNFR-alpha1; GFR-ALPHA-1); U95847; BC014962; NM_145793 NM_005264; Kim, M. H. et al. (2009) Mol. Cell. Biol. 29 (8), 2264-2277; Treanor, J. J. et al. (1996) Nature 382 (6586):80-83.

(40) Ly6E (lymphocyte antigen 6 complex, locus E; Ly67,RIG-E,SCA-2,TSA-1); NP_002337.1; NM_002346.2; de Nooij-van Dalen, A. G. et al. (2003) Int. J. Cancer 103 (6), 768-774; Zammit, D. J. et al. (2002) Mol. Cell. Biol. 22 (3):946-952.

(41) TMEM46 (shisa homolog 2 (Xenopus laevis); SHISA2); NP_001007539.1; NM_001007538.1; Furushima, K. et al. (2007) Dev. Biol. 306 (2), 480-492; Clark, H. F. et al. (2003) Genome Res. 13 (10):2265-2270.

(42) Ly6G6D (lymphocyte antigen 6 complex, locus G6D; Ly6-D, MEGT1); NP_067079.2; NM_021246.2; Mallya, M. et al. (2002) Genomics 80 (1):113-123; Ribas, G. et al. (1999) J. Immunol. 163 (1):278-287.

(43) LGR5 (leucine-rich repeat-containing G protein-coupled receptor 5; GPR49, GPR67); NP_003658.1; NM_003667.2; Salanti, G. et al. (2009) Am. J. Epidemiol. 170 (5):537-545; Yamamoto, Y. et al. (2003) Hepatology 37 (3):528-533.

(44) RET (ret proto-oncogene; MEN2A; HSCR1; MEN2B; MTC1; PTC; CDHF12; Hs.168114; RET51; RET-ELE1); NP_066124.1; NM_020975.4; Tsukamoto, H. et al. (2009) Cancer Sci. 100 (10):1895-1901; Narita, N. et al. (2009) Oncogene 28 (34):3058-3068.

(45) LY6K (lymphocyte antigen 6 complex, locus K; LY6K; HSJ001348; FLJ35226); NP_059997.3; NM_017527.3; Ishikawa, N. et al. (2007) Cancer Res. 67 (24):11601-11611; de Nooij-van Dalen, A. G. et al. (2003) Int. J. Cancer 103 (6):768-774.

(46) GPR19 (G protein-coupled receptor 19; Mm.4787); NP_006134.1; NM_006143.2; Montpetit, A. and Sinnett, D. (1999) Hum. Genet. 105 (1-2):162-164; O'Dowd, B. F. et al. (1996) FEBS Lett. 394 (3):325-329.

(47) GPR54 (KISS1 receptor; KISS1R; GPR54; HOT7T175; AXOR12); NP_115940.2; NM_032551.4; Navenot, J. M. et al. (2009) Mol. Pharmacol. 75 (6):1300-1306; Hata, K. et al. (2009) Anticancer Res. 29 (2):617-623.

(48) ASPHD1 (aspartate beta-hydroxylase domain containing 1; LOC253982); NP_859069.2; NM_181718.3; Gerhard, D. S. et al. (2004) Genome Res. 14 (10B):2121-2127.

(49) Tyrosinase (TYR; OCAIA; OCA1A; tyrosinase; SHEP3); NP_000363.1; NM_000372.4; Bishop, D. T. et al. (2009) Nat. Genet. 41 (8):920-925; Nan, H. et al. (2009) Int. J. Cancer 125 (4):909-917.

(50) TMEM118 (ring finger protein, transmembrane 2; RNFT2; FLJ14627); NP_001103373.1; NM_001109903.1; Clark, H. F. et al. (2003) Genome Res. 13 (10):2265-2270; Scherer, S. E. et al. (2006) Nature 440 (7082):346-351.

(51) GPR172A (G protein-coupled receptor 172A; GPCR41; FLJ11856; D15Ertd747e); NP_078807.1; NM_024531.3; Ericsson, T. A. et al. (2003) Proc. Natl. Acad. Sci. U.S.A. 100 (11):6759-6764; Takeda, S. et al. (2002) FEBS Lett. 520 (1-3):97-101.

(52) CD33, a member of the sialic acid binding, immunoglobulin-like lectin family, is a 67-kDa glycosylated transmembrane protein. CD33 is expressed on most myeloid and monocytic leukemia cells in addition to committed myelomonocytic and erythroid progenitor cells. It is not seen on the earliest pluripotent stem cells, mature granulocytes, lymphoid cells, or nonhematopoietic cells (Sabbath et al., (1985) J. Clin. Invest. 75:756-56; Andrews et al., (1986) Blood 68:1030-5). CD33 contains two tyrosine residues on its cytoplasmic tail, each of which is followed by hydrophobic residues similar to the immunoreceptor tyrosine-based inhibitory motif (ITIM) seen in many inhibitory receptors.

(53) CLL-1 (CLEC12A, MICL, and DCAL2), encodes a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. The protein encoded by this gene is a negative regulator of granulocyte and monocyte function. Several alternatively spliced transcript variants of this gene have been described, but the full-length nature of some of these variants has not been determined. This gene is closely linked to other CTL/CTLD superfamily members in the natural killer gene complex region on chromosome 12p13 (Drickamer K (1999) Curr. Opin. Struct. Biol. 9 (5):585-90; van Rhenen A, et al., (2007) Blood 110 (7):2659-66; Chen C H, et al. (2006) Blood 107 (4):1459-67; Marshall A S, et al. (2006) Eur. J. Immunol. 36 (8):2159-69; Bakker A B, et al. (2005) Cancer Res. 64 (22):8443-50; Marshall A S, et al. (2004) J. Biol. Chem. 279 (15):14792-802). CLL-1 has been shown to be a type II transmembrane receptor comprising a single C-type lectin-like domain (which is not predicted to bind either calcium or sugar), a stalk region, a transmembrane domain and a short cytoplasmic tail containing an ITIM motif.

“Antibody-drug conjugate” (ADC) is a targeted anti-cancer therapeutic designed to reduce nonspecific toxicities and increase efficacy relative to conventional small molecule and antibody cancer chemotherapy. They employ the targeting ability of monoclonal antibodies to deliver potent, conjugated small molecule therapeutics to a cancer cell. Antibody-drug conjugates structurally comprise an antibody covalently attached to one or more drug moieties through a linker. The ADC undergoes cleavage to release a cell-killing agent. The antibody portion of the ADC may be an antibody which binds to one or more tumor-associated antigens (TAA) or cell-surface receptors selected from (1)-(53) described herein.

The term “BPA” refers to a p-benzoyl-L-phenylalanine moiety having the structure:

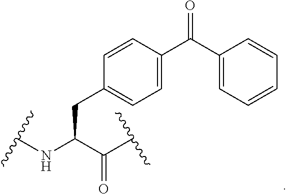 [SMILES: *N[C@@H](Cc1ccc(C(=O)c2ccccc2)cc1)C(*)=O]

The terms “PhL,” “photo-Leu,” “L-photo-leucine,” and “PhoLeu” are used interchangeably herein and refer to a diazirinyl leucine moiety having the structure:

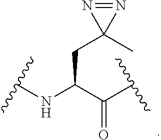 [SMILES: *N[C@@H](CC1(C)N=N1)C(*)=O]

The term “Tdf” refers to a 3-trifluoromethyl-3-phenyldiazarine moiety having the structure:

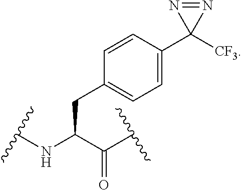 [SMILES: *N[C@@H](Cc1ccc(C2(C(F)(F)F)N=N2)cc1)C(*)=O]

The terms “PhM” and “photo-methionine” are used interchangeably herein and refer to a diazirinyl methionine moiety having the structure:

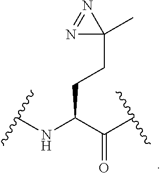 [SMILES: *N[C@@H](CCC1(C)N=N1)C(*)=O]

The term “photoactivatable amino acid residue” refers to a non-naturally occurring, UV-activated, cross-linking amino acid within a peptide. Peptides containing a BPA photoactivatable amino acid residue are referred to herein as “BPA peptides”. Peptides containing a PhL photoactivatable amino acid residue are referred to herein as “PhL peptides”. Peptides containing a Tdf photoactivatable amino acid residue are referred to herein as or “Tdf peptides”. Peptides containing a PhM photoactivatable amino acid residue are referred to herein as or “PhM peptides”. A composition comprising one or more BPA peptides is referred to herein as a BPA peptide composition. A composition comprising one or more PhL peptides is referred to herein as a PhL peptide composition. A composition comprising one or more Tdf peptides is referred to herein as a Tdf peptide composition.

The termS “photocrosslink” AND “photoconjugate” refer to the photoinduced formation of a covalent bond between two macromolecules such as a protein or peptide, or between two different parts of one macromolecule. “Photo-crosslinking conditions” refer to parameters such as those described herein that facilitate or enhance photocrosslinking (e.g. light wavelength, antioxidants, buffers, temperature).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). In one embodiment, where BPA peptides are conjugated as described herein, the interaction can be a 2:2 interaction where there is one peptide per side of the symmetric Fc domain). The affinity of a molecule X for its partner Y can generally be represented by the dissociation constant (K_{d}). Affinity can be measured by common methods known in the art, including those described herein. Specific illustrative and exemplary embodiments for measuring binding affinity are known in the art, any of which can be used for purposes of the present invention. The K_{d} or K_{d} value may be measured by using surface plasmon resonance assays using a system such a BIAcore™-2000 or a BIAcore™-3000 instrument (BIAcore, Inc., Piscataway, N.J.).

Direct methods to prepare antibody-drug conjugates from wild-type antibodies that do not require engineering or modification of the antibody are needed. Such methods could, for example, enable the generation of homogeneous ADCs in as few as one chemical step, greatly simplifying the conjugation process as set forth herein. Furthermore, interchain disulfides and glycans can remain intact with such approaches, maximizing biological activity dependent on these features. When combined with chemically orthogonal conjugation approaches (e.g., involving mutation of the antibody sequence, as described herein), methods for modifying wild-type antibodies can enable the construction of ADCs with two or more different payloads with defined stoichiometries for each payload.

Provided herein are BPA peptides and compositions comprising BPA peptide BPA1-BPA-10 as set forth in Table 1. In one embodiment, the BPA peptide composition comprises BPA3 or BPA4. In one embodiment, the BPA peptide comprises BPA7 (i.e. SEQ ID NO:8). In one embodiment, the BPA peptide composition comprises BPA10.

Further provided herein are PhL peptides and compositions comprising PhL peptide PhL1-PhL9 as set forth in Table 2. Still further provided herein are Tdf peptide compositions selected from the group consisting of Tdf1-Tdf9 as set forth in Table 3.

The BPA peptides described herein can be synthesized using solid-phase peptide synthesis methods (SPPS), including those known in the art. In one embodiment, BPA peptides synthesized using SPPS have less than about 5%, 3%, 1%, 0.5%, 0.3%, 0.1%, 0.05% or 0.01% impurities. In one embodiment, BPA peptides described herein can be synthesized in accordance with the Examples as set forth herein. BPA peptides described herein that allow for subsequent modification with a payload were made by SPPS and then modified chemically post-cleavage with an extension moiety as described herein. Extension moieties useful in the ADCs and methods herein include, for example, groups having one or more thiols, azides, tetrazines, cycloalkynes, or other group allowing click chemistry post-photoconjugation. In one embodiment, the extension moiety is:

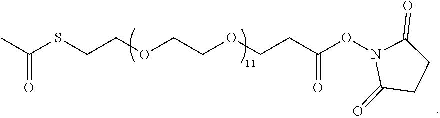

TABLE 1
Fc-III Peptide and BPA peptide sequences
(N-Ac, C-amide)

| Peptide | Sequence | SEQ ID |
|---|---|---|
| Fc-III | Ac-DCAWHLGELVWCT-NH₂ | SEQ ID NO: 1 |
| BPA1 | Ac-BCAWHLGELVWCT-NH₂ | SEQ ID NO: 2 |
| BPA2 | Ac-DCBWHLGELVWCT-NH₂ | SEQ ID NO: 3 |
| BPA3 | Ac-DCAWBLGELVWCT-NH₂ | SEQ ID NO: 4 |
| BPA4 | Ac-DCAWHBGELVWCT-NH₂ | SEQ ID NO: 5 |
| BPA5 | Ac-DCAWHLGBLVWCT-NH₂ | SEQ ID NO: 6 |
| BPA6 | Ac-DCAWHLGEBVWCT-NH₂ | SEQ ID NO: 7 |
| BPA7 | Ac-DCAWHLGELBWCT-NH₂ | SEQ ID NO: 8 |
| BPA8 | Ac-DCAWHLGELVBCT-NH₂ | SEQ ID NO: 9 |
| BPA9 | Ac-DCAWHLGELVWCB-NH₂ | SEQ ID NO: 10 |
| BPA10 | Ac-CDCAWHLGELBWCTC-NH2 | SEQ ID NO: 11 |

B = BPA

TABLE 2
PhL peptide sequences (N-Ac, C-amide)

| Peptide | Sequence | SEQ ID |
|---|---|---|
| PhL1 | Ac-XCAWHLGELVWCT-NH₂ | SEQ ID NO: 12 |
| PhL2 | Ac-DCXWHLGELVWCT-NH₂ | SEQ ID NO: 13 |
| PhL3 | Ac-DCAWXLGELVWCT-NH₂ | SEQ ID NO: 14 |
| PhL4 | Ac-DCAWHXGELVWCT-NH₂ | SEQ ID NO: 15 |
| PhL5 | Ac-DCAWHLGXLVWCT-NH₂ | SEQ ID NO: 16 |
| PhL6 | Ac-DCAWHLGEXVWCT-NH₂ | SEQ ID NO: 17 |
| PhL7 | Ac-DCAWHLGELXWCT-NH₂ | SEQ ID NO: 18 |
| PhL8 | Ac-DCAWHLGELVXCT-NH₂ | SEQ ID NO: 19 |
| PhL9 | Ac-DCAWHLGELVWCX-NH₂ | SEQ ID NO: 20 |

X = PhL

TABLE 3
Tdf peptide sequences (N-Ac, C-amide)

| Peptide | Sequence | SEQ ID |
|---|---|---|
| Tdf1 | Ac-ZCAWHLGELVWCT-NH₂ | SEQ ID NO: 21 |
| Tdf2 | Ac-DCZWHLGELVWCT-NH₂ | SEQ ID NO: 22 |
| Tdf3 | Ac-DCAWZLGELVWCT-NH₂ | SEQ ID NO: 23 |
| Tdf4 | Ac-DCAWHZGELVWCT-NH₂ | SEQ ID NO: 24 |
| Tdf5 | Ac-DCAWHLGZLVWCT-NH₂ | SEQ ID NO: 25 |
| Tdf6 | Ac-DCAWHLGEZVWCT-NH₂ | SEQ ID NO: 26 |
| Tdf7 | Ac-DCAWHLGELZWCT-NH₂ | SEQ ID NO: 27 |
| Tdf8 | Ac-DCAWHLGELVZCT-NH₂ | SEQ ID NO: 28 |
| Tdf9 | Ac-DCAWHLGELVWCZ-NH₂ | SEQ ID NO: 29 |

Z = Tdf

The Fc-III peptide, (SEQ ID NO:1, Table 1) binds to the Fc fragment of human immunoglobulin G (IgG) at a consensus site between the CH2 and CH3 domains (DeLano, W. L. et al (2000) Science 287:1279-1283) with nanomolar affinity.

In one embodiment, BPA peptides described herein further comprising an extension moiety attached to the C-terminal amide. In one embodiment, the extension moiety comprises S-acetylthioacetate (SATA) having the structure:

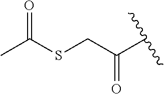 [SMILES: *C(=O)CSC(C)=O]

In one embodiment, the extension moiety comprises an azide, a cyclooctyne, or a tetrazinyl moiety of the structure:

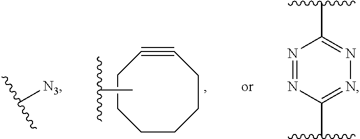

In one embodiment, a BPA peptide is biotinylated. In one embodiment, a BPA peptide is attached to a fluorophore.

In one embodiment, the BPA peptides described herein further comprise an extension moiety comprising one or more repeating PEG units:

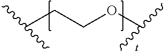

where t=2-40.

In one embodiment, the extension moiety comprises 2-40, 2-30, 2-25, 2-20, 2-15, 2-12, or 2-10 PEG units. In one embodiment, the extension moiety comprised PEG₂, PEG₃, PEG₄, PEG₅, PEG₆, PEG₇, PEG₈, PEG₉, PEG₁₀, PEG₁₁, PEG₁₂, PEG₁₃, PEG₁₄, PEG₁₅, PEG₁₆, PEG₁₇, PEG₁₈, PEG₁₉, or PEG₂₀. In one embodiment, the BPA peptides described herein include an extension moiety comprising SATA-PEG₍₂₋₁₂₎. In one embodiment, the BPA peptides described herein include an extension moiety comprising SATA-PEG₁₂.

The affinity of the BPA peptides described herein for the Fc fragment of IgG (i.e. the K_{d}) can be measure using techniques understood in the art such as, for example, surface plasmon resonance (SPR). In one embodiment, BPA peptides described herein have a K_{d} of about 0.01 μM to about 100 μM, about 0.01 μM to about 70 μM, about 0.01 μM to about 50 μM, about 0.01 μM to about 25 μM, about 0.01 μM to about 10 μM, about 0.01 μM to about 5 μM, about 0.01 μM to about 1 μM, or about 0.01 μM to about 0.5 μM. In another embodiment, BPA peptides described herein have a K_{d} of about 0.5 μM to about 70 μM, about 0.5 μM to about 50 μM, or about 0.5 μM to about 10 μM. In another embodiment, BPA peptides described herein have a K_{d} of about 10 μM to about 75 μM, about 15 μM to about 75 μM, about 25 μM to about 75 μM, or about 50 μM to about 75 μM. In still another embodiment, BPA peptides described herein have a K_{d} of about 50 μM to about 100 μM. In one embodiment, BPA peptides described herein have a K_{d} of about 0.5, 1, 5, 10, 15, 25, 30, 50, 70, or about 80 μM.

The affinity of the BPA peptides described herein can also be compared to the affinity of the Fc-III peptide. In one embodiment, the K_{d} of a BPA peptide described herein is reduced when compared to the Fc-III peptide. In one embodiment, the K_{d} of a BPA peptide described herein is between 25-4200-fold decreased comparable to the Fc-III peptide. In one embodiment, the K_{d} of a BPA peptide described herein is greater than about 4000-fold decreased comparable to the Fc-III peptide. In one embodiment, the K_{d} of a BPA peptide described herein is greater than about 4000-fold decreased comparable to the Fc-III peptide.

In one embodiment, the BPA peptide comprises BPA7 (SEQ ID NO:8) as described herein and has a K_{d} of about 70 μM. In one embodiment, the BPA peptide comprises BPA7 as described herein and has a K_{d} that is greater than about 4000-fold decreased comparable to the Fc-III peptide.

In one embodiment, the BPA peptide comprises BPA10 (SEQ ID NO:11) as described herein and has a K_{d} of about 11 μM. In one embodiment, the BPA peptide comprises BPA10 as described herein and has a K_{d} that is greater than about 600-fold decreased comparable to the Fc-III peptide.

In one embodiment, the BPA peptide comprises BPA4 (SEQ ID NO:11) as described herein and has a K_{d} of about 30 μM. In one embodiment, the BPA peptide comprises BPA4 as described herein and has a K_{d} that is greater than about 1700-fold decreased comparable to the Fc-III peptide.

BPA peptides described herein can be attached to an antibody having a methionine at a corresponding 252 position (Met-252 as described herein). In one embodiment, the antibody is a human IgG antibody comprising Met-252. In one embodiment, BPA peptides can be attached to a therapeutic antibody For example, in one embodiment, the therapeutic antibody comprises a therapeutic antibody selected from the group consisting of mogamulizumab, blinatumomab, rituximab, ofatumumab, obinutuzumab, ibritumomab, tositumomab, inotuzumab, brentuximab vedotin, gemtuzumab ozogamicin, daratumumab, ipilimumab, cetuximab, panitumumab, necitumumab, minotuzumab, dinutuximab, trastuzumab, pertuzumab, ado-trastuzumab emtansine, nivolumab, pembrolizumab, atezolizumab, avelumab, durvalumab, olaratumab, denosumab, elotuzumab, bevacizumab, or ramucirumab.

In one preferred embodiment, the therapeutic antibody comprises a therapeutic antibody selected from the group consisting of rituximab, obinutuzumab, trastuzumab, pertuzumab, ado-trastuzumab emtansine, or bevacizumab. In one embodiment, the therapeutic is trastuzumab (HERCEPTIN®) or trastuzumab emtansine (KADCYLA®). In one embodiment, the therapeutic is trastuzumab (HERCEPTIN®).

In one embodiment, the therapeutic antibody comprises gemtuzumab ozogamicin. In one embodiment, the therapeutic antibody comprises ipilimumab. In one embodiment, the therapeutic antibody comprises daratumumab. In one embodiment, the therapeutic antibody comprises cetuximab. In one embodiment, the therapeutic antibody comprises nivolumab. In one embodiment, the therapeutic antibody comprises pembrolizumab. In one embodiment, the therapeutic antibody comprises avelumab. In one embodiment, the therapeutic antibody comprises durvalumab. In one embodiment, the therapeutic antibody comprises rituximab. In one embodiment, the therapeutic antibody comprises obinutuzumab. In one embodiment, the therapeutic antibody comprises trastuzumab. In one embodiment, the therapeutic antibody comprises pertuzumab. In one embodiment, the therapeutic antibody comprises ado-trastuzumab emtansine. In one embodiment, the therapeutic antibody comprises bevacizumab.

In another embodiment, the therapeutic antibody comprises a therapeutic antibody selected from the group consisting of natalizumab, vedolizumab, belimumab, itolizumab, ocrelizumab, alemtuzumab, omalizumab, canakinumab, daclizumab, dupilumab, reslizumab, mepolizumab, benralizumab, sirukumab, siltuximab, sarilumab, tocilizumab, ustekinumab, ixekizumab, secukinumab, brodalumab, guselkumab, tildrakizumab, infliximab, adalimumab, certolizumab, golimumab.

In one preferred embodiment, the therapeutic antibody comprises ocrelizumab, omalizumab, or tocilizumab. Infliximab. In one embodiment, the therapeutic antibody comprises natalizumab. In one embodiment, the therapeutic antibody comprises adalimumab.

In another embodiment, the therapeutic antibody comprises a therapeutic antibody selected from the group consisting of eculizumab, idarucizumab, emicizumab, abciximab, alirocumab, evolocumab, capalacizumab. In one embodiment, the therapeutic antibody comprises emicizumab.

In still another embodiment, the therapeutic antibody comprises a therapeutic antibody selected from the group consisting of raxibacumab, obiltoxaximab, ibalizumab, bezlotoxumab, or palivizumab.

In still another embodiment, the therapeutic antibody comprises a therapeutic antibody selected from the group consisting of ranibizumab. In another embodiment, the therapeutic antibody comprises a therapeutic antibody selected from the group consisting of muromonab-CD3, romosozumab, erenumab, burosumab,

In another embodiment, the BPA peptides described herein are attached to a non-human antibody that contains Met-252 residue.

In one embodiment, the BPA peptides described herein are attached to a HER2 specific antibody for the treatment or management of a HER2-related cancer. In one embodiment, the BPA peptides described herein are attached to a PD-1 or PD-L1 specific antibody for the treatment or management of a PD-1 or PD-L1 related cancer.

Further provided herein are antibody-drug conjugates (ADC) comprising a BPA peptide described herein attached to the Fc portion of an antibody (Ab) described herein. The ADC further comprises a linker moiety (L) as described herein attached to a drug moiety (D) as described herein.

In one embodiment, the antibody-drug conjugate is a composition comprising a BPA peptide described herein, an antibody described herein, L, and D as set forth herein. In one embodiment, the ADC comprises Formula (I):

Ab [symbol] B-E-L-D)ₚ (I)
wherein:
Ab is an antibody as described herein;
B is a BPA peptide as described herein (e.g. BPA1-BPA10) covalently attached to the Fc region of the antibody and to the linker (L);
E is an optional extension moiety as provided herein;
L is an optional linker as provided herein;
D is a drug moiety comprising a radiolabel, an antibody, or an anti-cancer agent such as a tubulin inhibitor, a topoisomerase II inhibitor, a DNA crosslinking cytoxic agent, an alkylating agent, a taxane, or an anthracycline agent; and
p is 1 or 2.

It is understood that p refers to the drug-to-antibody ratio or “DAR”. In one embodiment, p is 1 (i.e. a DAR of 1). In one embodiment, p is 2 (i.e. a DAR of 2). It is understood that p (and DAR) refer to the ratio (drug-to-antibody) of the composition. Thus, in some embodiments, the calculated DAR may be a non-integer value of approximately 2 (e.g. 1.5, 1.6, 1.7, 1.8, 1.9, 2.1, or 2.2, including values therein). Likewise, in some embodiments, the calculated DAR may be a non-integer value of approximately 1 (e.g. 0.3, 0.4, 0.5, 0.6, 0.7, 0.8, 0.9, 1.1, or 1.2, including values therein).

In one embodiment, D is a maytansinoid, dolastatin, auristatin, calicheamicin, pyrrolobenzodiazepine dimer (PBD dimer), an anthracycline agent, duocarmycin, a synthetic duocarmycin analogue, a 1,2,9,9a-Tetrahydrocyclopropa[c]benzo[e]indol-4-one (CBI) dimer, a vinca alkaloid, a taxane (e.g. paclitaxel or docetaxel), trichothecene, camptothecin, silvestrol, or elinafide.

In one embodiment, the duocarmycin is mycarosylprotylonolide (CC1065). In one embodiment, the synthetic duocarmycin analogue is adozelesin, bizelesin, or carzelesin.

In one embodiment, D is a dolastatin (such as those moieties provided in WO 2015/090050; (U.S. Pat. Nos. 5,635,483; 5,780,588; 5,767,237; and 6,124,431, each of which is herein incorporated by reference in its entirety and for all purposes).

In one embodiment, D is a PBD dimer (such as those PBD dimer moieties provided in WO 2017/064675; WO 2015/095124; WO 2017/059289; WO 2014/159981; and EP2528625, each of which is herein incorporated by reference in its entirety and for all purposes).

In one embodiment, D is a PBD dimer having the structure:

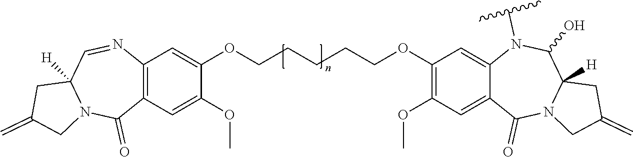

wherein n is 0 or 1 and the antibody is attached through a linker as described herein at the position of the wavy line.

In one embodiment, an ADC described herein comprises a linker drug comprising the formula (II):

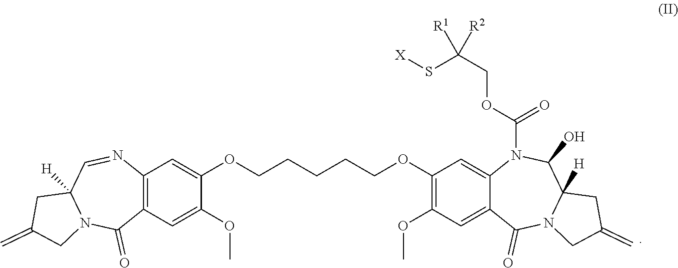

where X is a pyridyl leaving group, and R¹ and R² are independently H or C₁-C₆ alkyl (e.g. methyl, ethyl, or propyl).

In one embodiment, D is a CBI dimer (such as those CBI dimer moieties provided in WO 2015/023355; WO 2015/095227, each of which is herein incorporated by reference in its entirety and for all purposes).

In one embodiment, D is an auristatin (such as those moieties provided in U.S. Pat. Nos. 7,498,298; 7,659,241; and WO 2002/088172, each of which is herein incorporated by reference in its entirety and for all purposes).

In one embodiment, where D is an auristatin, the auristatin is MMAE having the structure;

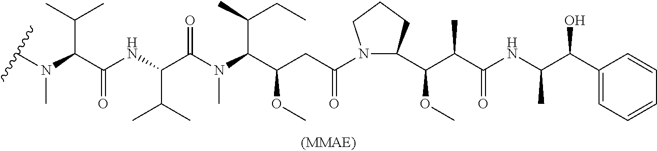 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates covalent attachment to L as described herein.

In one embodiment, where D is an auristatin, the auristatin is MMAF.

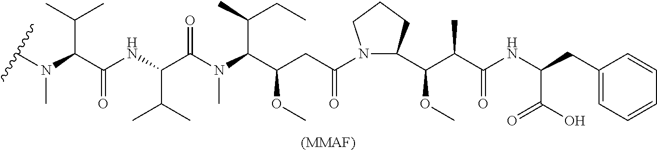

wherein the wavy line indicates covalent attachment to L as described herein.

In one embodiment, D is a maytansinoid (such as those moieties provided in U.S. Pat. Nos. 5,208,020 and 5,416,064; and US 2005/0276812, each of which is herein incorporated by reference in its entirety and for all purposes).

In one embodiment, D is an anthracycline agent comprising PNU-159682, doxorubicin, daunorubicin, epirubicin, idarubicin, mitoxantrone, or valrubicin. In one embodiment, the anthracycline agent is PNU-159682.

In one embodiment, the vinca alkaloid is vinblastine, vincristine, vindesine, or vinorelbine.

In one embodiment, D is a calicheamicin compound having formula (III):

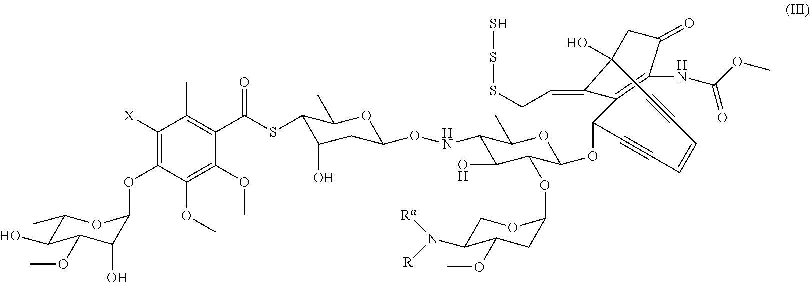

wherein X is Br or I; L is a linker as provided herein; R is hydrogen, C₁₋₆ alkyl, or —C(═O) C₁₋₆alkyl; and Ra is hydrogen or C₁₋₆alkyl. Many positions on calicheamicin compounds are useful as the linkage position. For example, an ester linkage may be formed by reaction with a hydroxyl group using conventional coupling techniques.

In one embodiment, D is a radiolabel such as, for example, ¹¹C, ¹³N, ¹⁵O, ¹⁸F, ³²P, ⁵¹Cr, ⁵⁷Co, ⁶⁴Cu, ⁶⁷Ga, ⁷⁵Se, ^{81m}Kr, ⁸²Rb, ^{99m}TC, ¹²³I, ¹²⁵I, ¹³¹I, ¹¹¹In, and ²⁰¹Ti.

In one embodiment, D is a fluorophore or label such as, for example, fluorescein, hydroxyl tratamine, rhodamine, coumarin, alexa fluor, bodipy, dansyl, GFP, YFP, digoxigenin, dinitrophenol, or biotin, including analogues and derivatives thereof.

E is an extension moiety as described herein. In one embodiment, the extension moiety comprises (SATA). In one embodiment, the BPA peptides described herein further comprise an extension moiety comprising one or more repeating PEG units:

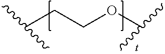

where t=2-40.

In one embodiment, the BPA peptides described herein include an extension moiety comprising SATA-PEG₍₂₋₁₂₎. In one embodiment, the BPA peptides described herein include an extension moiety comprising SATA-PEG₁₂.

L can be a bifunctional or multifunctional moiety used to link one or more drug moieties (D) to the BPA peptide described herein to form an ADC as set forth herein. In one embodiment, L is a self-immolative linker comprising at least one of a disulfide moiety, a peptide moiety or a peptidomimetic moiety.

In one embodiment, L has the formula (IV):

-Str-(Pep)ₘ-(Y)ₙ- (IV)
wherein,
Str is a stretcher unit or S covalently attached the BPA peptide;
Pep is an optional peptide unit of two to twelve amino acid residues;
Y is an optional spacer unit covalently attached to D; and
m and n are independently selected from 0 and 1.

In one embodiment, Str comprises a maleimidyl, bromacetamidyl, iodoacetamidyl, moiety. In one embodiment, Str comprises a reactive disulfide group such as those set forth in US Patent Application No. 2017-0112891, which is herein incorporated by reference in its entirety and for all purposes.

In one embodiment, L comprises formula (IV) wherein Str has the formula (V):

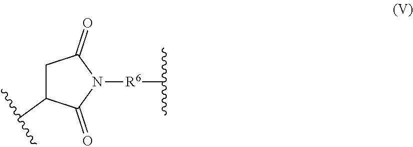

wherein,
R⁶ comprises C₁-C₁₂ alkylene, C₁-C₁₂ alkylene-C(═O), C₁-C₁₂ alkylene-NH, (CH₂CH₂O)ᵣ, (CH₂CH₂O)ᵣ—C(═O), (CH₂CH₂O)ᵣ—CH₂, or C₁-C₁₂ alkylene-NHC(═O)CH₂CH (thiophen-3-yl);
r is an integer ranging from 1 to 12; and
R⁶ is attached to Pep or Y.

In one embodiment, R⁶ is (CH₂)₅.

In one embodiment, R⁶ comprises PEG (e.g. PEG₁₀ or PEG₁₂).

Pep can comprise natural amino acids or non-proteinogenic amino acids.

In some embodiments, L comprises formula (IV), where Str is as defined herein and Pep is a self-immolative peptide moiety cleaved by enzymatic cleavage, such as by a protease, thereby facilitating release of the drug from the immunoconjugate upon exposure to intracellular proteases, such as lysosomal enzymes (Doronina et al. (2003) Nat. Biotechnol. 21:778-784). Exemplary peptide units include, but are not limited to, dipeptides, tripeptides, tetrapeptides, and pentapeptides. Exemplary dipeptides include, but are not limited to, valine-citrulline (vc or val-cit), valine-alanine (va or val-ala), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). A peptide unit may comprise amino acid residues that occur naturally and/or minor amino acids and/or non-naturally occurring amino acid analogs, such as citrulline. Peptide units can be designed and optimized for enzymatic cleavage by a particular enzyme, for example, a tumor-associated protease, cathepsin B, C and D, or a plasmin protease.

In some embodiments, L comprises formula (IV), where Str is as defined herein and Pep is a self-immolative peptidomimetic moiety. Exemplary peptidomimetic units include, but are not limited to, triazoles, cyclobutane-1-1-dicarbaldehyde, cyclobutane-1-1-dicarbaldehyde-citrulline, alkenes, haloalkenes, and isoxazoles.

In one embodiment, Pep is a self-immolative peptidomimetic moiety comprising one or more of the moieties:

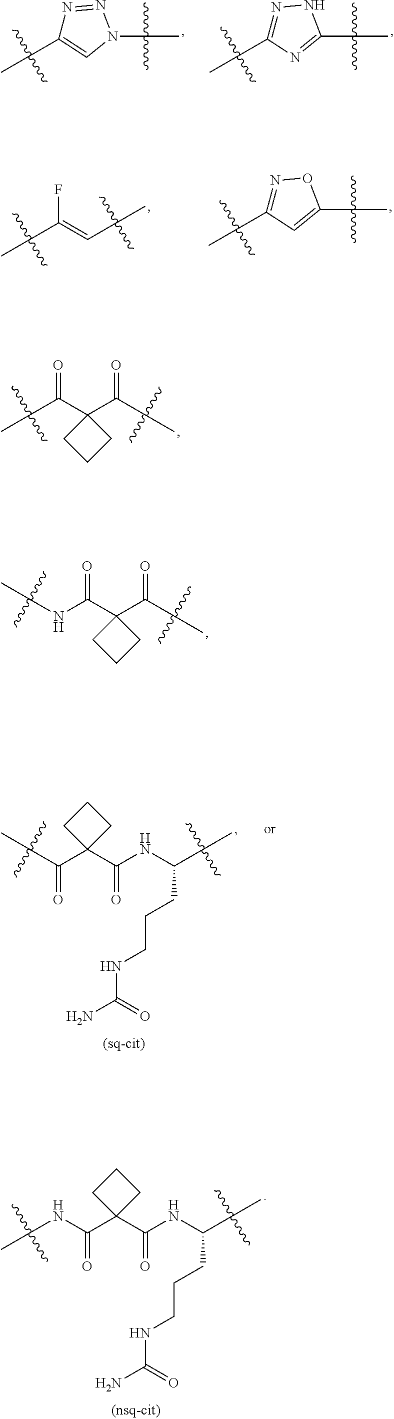

where the wavy line at the left side of the peptidomimetic moiety is the point of connection to a Str and the wavy line at the right side of the peptidomimetic moiety is the point of connection to D.

In one preferred embodiment, the peptidomimetic moiety comprises:

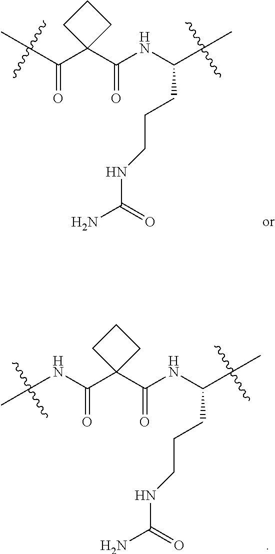 [SMILES: *C(=O)C1(C(=O)N[C@H](*)CCCNC(N)=O)CCC1 ; *NC(=O)C1(C(=O)N[C@H](*)CCCNC(N)=O)CCC1]

In one embodiment, Pep comprises two to twelve amino acid residues independently selected from the group consisting of glycine, alanine, phenylalanine, lysine, arginine, valine, and citrulline.

In one embodiment, Pep comprises valine-citrulline, alanine-phenylalanine, or phenylalanine-lysine.

In one embodiment, Pep comprises sq-cit or nsq-cit as described herein.

In one embodiment of formula (IV), Str is S, Pep is as defined herein, and Y comprises para-aminobenzyl or para-aminobenzyloxycarbonyl.

In one preferred embodiment, L comprises formula (IV) where R₆ is (CH₂)₅, Pep is val-cit, sq-cit, or nsq-cit, and Y is PAB. In another preferred embodiment, L comprises formula (IV) where R₆ is PEG (e.g. PEG₁₂), Pep is val-cit, sq-cit, or nsq-cit, and Y is PAB. In one embodiment of the above, Pep is val-cit. In one embodiment of the above, Pep is sq-cit or nsq-cit.

In some embodiments, L comprises a self-immolative disulfide.

In one embodiment, L has the formula (VI):

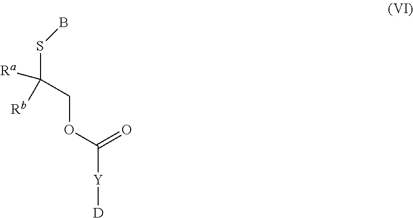

wherein,
B and D are as defined herein; and
Y is para-aminobenzyl, p-aminobenzyloxycarbonyl (PAB), 2-aminoimidazol-5-methanol derivatives, ortho- or para-aminobenzylacetals, 4-aminobutyric acid amides, bicyclo[2.2.1] and bicyclo[2.2.2] ring systems, or 2-aminophenylpropionic acid amides; and
R^{a} and R^{b} are independently selected from H and C₁₋₃ alkyl, wherein only one of R^{a} and R^{b} can be H, or R^{a} and R^{b} together with the carbon atom to which they are bound form a four- to six-membered ring optionally comprising an oxygen heteroatom.

In one embodiment, R^{a} and R^{b} are independently selected from H, —CH₃ and —CH₂CH₃, wherein only one of R^{a} and Rb can be H, or R^{a} and Rb together with the carbon atom to which they are bound form a ring selected from cyclobutyl, cyclopentyl, cyclohexyl, tetrahydrofuran and tetrahydropyran.

In one embodiment, Y is para-aminobenzyl or p-aminobenzyloxycarbonyl.

In one preferred embodiment, Y comprises p-aminobenzyloxycarbonyl (PAB). In some such embodiments, a Y can be attached to an amino acid unit via an amide bond, and a carbamate, methylcarbamate, or carbonate connection is made between the benzyl alcohol and the drug (Hamann et al. (2005) Expert Opin. Ther. Patents (2005) 15:1087-1103).

In one embodiment Y comprises 2-aminoimidazol-5-methanol derivatives (such as those set forth in U.S. Pat. No. 7,375,078; Hay et al. (1999) Bioorg. Med. Chem. Lett. 9:2237, each of which is hereby incorporated by reference in its entirety and for all purposes).

In one embodiment, Y undergo cyclization upon amide bond hydrolysis. In such embodiments Y can be a substituted and unsubstituted 4-aminobutyric acid amide (such as those described by Rodrigues et al (1995) Chemistry Biology 2:223, which is hereby incorporated by reference in its entirety and for all purposes), a substituted bicyclo[2.2.1] and bicyclo[2.2.2] ring system (such as those described by Storm et al (1972) J. Amer. Chem. Soc. 94:5815, which is hereby incorporated by reference in its entirety and for all purposes), or a 2-aminophenylpropionic acid amide (such as those described by Amsberry, et al (1990) J. Org. Chem. 55:5867, which is hereby incorporated by reference in its entirety and for all purposes).

In one embodiment, the antibody described herein binds to a tumor-associated antigen or cell-surface receptor selected from the group consisting of those numbered (1)-(53) below:
(1) BMPR1B (bone morphogenetic protein receptor-type IB);
(2) E16 (LAT1, SLC7A5);
(3) STEAP1 (six transmembrane epithelial antigen of prostate);
(4) MUC16 (0772P, CA125);
(5) MPF (MPF, MSLN, SMR, megakaryocyte potentiating factor, mesothelin);
(6) Napi2b (NAPI-3B, NPTIIb, SLC34A2, solute carrier family 34 (sodium phosphate), member 2, type II sodium-dependent phosphate transporter 3b);
(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B);
(8) PSCA hlg (2700050C12Rik, C530008O16Rik, RIKEN cDNA 2700050C12, RIKEN cDNA 2700050C12 gene);
(9) ETBR (Endothelin type B receptor);
(10) MSG783 (RNF124, hypothetical protein FLJ20315);
(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein);
(12) TrpM4 (BR22450, FLJ20041, TRPM4, TRPM4B, transient receptor potential cation channel, subfamily M, member 4);
(13) CRIPTO (CR, CR1, CRGF, CRIPTO, TDGF1, teratocarcinoma-derived growth factor);
(14) CD21 (CR2 (Complement receptor 2) or C3DR (C3d/Epstein Barr virus receptor) or Hs 73792);
(15) CD79b (CD79B, CD79β, IGb (immunoglobulin-associated beta), B29);
(16) FcRH2 (IFGP4, IRTA4, SPAP1A (SH2 domain containing phosphatase anchor protein 1a), SPAP1B, SPAP1C);
(17) HER2;
(18) NCA;
(19) MDP;
(20) IL20Rα;
(21) Brevican;
(22) EphB2R;
(23) ASLG659;
(24) PSCA;
(25) GEDA;
(26) BAFF-R (B cell-activating factor receptor, BLyS receptor 3, BR3);
(27) CD22 (B-cell receptor CD22-B isoform);
(28) CD79a (CD79A, CD79α, immunoglobulin-associated alpha);
(29) CXCR5 (Burkitt's lymphoma receptor 1);
(30) HLA-DOB (Beta subunit of MHC class II molecule (1a antigen));
(31) P2X5 (Purinergic receptor P2X ligand-gated ion channel 5);
(32) CD72 (B-cell differentiation antigen CD72, Lyb-2);
(33) LY64 (Lymphocyte antigen 64 (RP105), type I membrane protein of the leucine rich repeat (LRR) family);
(34) FcRH1 (Fc receptor-like protein 1);
(35) FcRH5 (IRTA2, Immunoglobulin superfamily receptor translocation associated 2);
(36) TENB2 (putative transmembrane proteoglycan);
(37) PMEL17 (silver homolog; SILV; D12S53E; PMEL17; SI; SIL);
(38) TMEFF1 (transmembrane protein with EGF-like and two follistatin-like domains 1; Tomoregulin-1);
(39) GDNF-Ra1 (GDNF family receptor alpha 1; GFRA1; GDNFR; GDNFRA; RETL1; TRNR1; RET1L; GDNFR-alpha1; GFR-ALPHA-1);
(40) Ly6E (lymphocyte antigen 6 complex, locus E; Ly67, RIG-E, SCA-2, TSA-1);
(41) TMEM46 (shisa homolog 2 (Xenopus laevis); SHISA2);
(42) Ly6G6D (lymphocyte antigen 6 complex, locus G6D; Ly6-D, MEGT1);
(43) LGR5 (leucine-rich repeat-containing G protein-coupled receptor 5; GPR49, GPR67);
(44) RET (ret proto-oncogene; MEN2A; HSCR1; MEN2B; MTC1; PTC; CDHF12; Hs.168114; RET51; RET-ELE1);
(45) LY6K (lymphocyte antigen 6 complex, locus K; LY6K; HSJ001348; FLJ35226);
(46) GPR19 (G protein-coupled receptor 19; Mm.4787);
(47) GPR54 (KISS1 receptor; KISS1R; GPR54; HOT7T175; AXOR12);
(48) ASPHD1 (aspartate beta-hydroxylase domain containing 1; LOC253982);
(49) Tyrosinase (TYR; OCAIA; OCA1A; tyrosinase; SHEP3);
(50) TMEM118 (ring finger protein, transmembrane 2; RNFT2; FLJ14627);
(51) GPR172A (G protein-coupled receptor 172A; GPCR41; FLJ11856; D15Ertd747e);
(52) CD33; and
(53) CLL-1.

In one embodiment, the antibody is an IgG antibody (human IgG or rabit IgG) comprising methionine (Met) at the position corresponding to 252. In one embodiment, where the antibody is an IgG antibody comprising Met252, the Met252 is not in an oxidized state. In one embodiment, the antibody is an IgG antibody not comprising mutations of Met252, Ser254, and T256.

In one embodiment, the Ab of the ADC is not an engineered antibody (e.g. an antibody lacking mutation of a residue to Cys).

In one embodiment, the Ab of the ADC retains its natural glycosylation following conjugation with a BPA peptide described herein.

In one embodiment, the Ab of the ADC is trastuzumab.

In one embodiment, the Ab of the ADC is trastuzumab emtansine.

In one embodiment, the Ab of the ADC is a THIOMAB™ antibody. In particular embodiments, the substituted residues occur at accessible sites of the antibody. By substituting those residues with cysteine, reactive thiol groups are thereby positioned at accessible sites of the antibody and may be used to conjugate the antibody to the drug moiety to create an ADC as described herein. In certain embodiments, any one or more of the following residues may be substituted with cysteine: V205 (Kabat numbering) of the light chain; K149 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In some embodiments, a THIOMAB™ antibody comprises one of the heavy or light chain cysteine substitutions listed in Table 4 below.

TABLE 4

| | | Screening | GNE | Kabat |
|---|---|---|---|---|
| Chain | | Mutation | Mutation | Mutation |
| (HC/LC) | Residue | Site # | Site # | Site # |
| LC | T | 22 | 22 | 22 |
| LC | K | 39 | 39 | 39 |
| LC | Y | 49 | 49 | 49 |
| LC | Y | 55 | 55 | 55 |
| LC | T | 85 | 85 | 85 |
| LC | T | 97 | 97 | 97 |
| LC | I | 106 | 106 | 106 |
| LC | R | 108 | 108 | 108 |
| LC | R | 142 | 142 | 142 |
| LC | K | 149 | 149 | 149 |
| LC | V | 205 | 205 | 205 |
| HC | T | 117 | 114 | 110 |
| HC | A | 143 | 140 | 136 |
| HC | L | 177 | 174 | 170 |
| HC | L | 182 | 179 | 175 |
| HC | T | 190 | 187 | 183 |
| HC | T | 212 | 209 | 205 |
| HC | V | 265 | 262 | 258 |
| HC | G | 374 | 371 | 367 |
| HC | Y | 376 | 373 | 369 |
| HC | E | 385 | 382 | 378 |
| HC | S | 427 | 424 | 420 |
| HC | N | 437 | 434 | 430 |
| HC | Q | 441 | 438 | 434 |

In other embodiments, a THIOMAB™ antibody comprises one of the heavy chain cysteine substitutions listed in Table 5.

TABLE 5

| | | Screening | GNE | Kabat |
|---|---|---|---|---|
| Chain | | Mutation | Mutation | Mutation |
| (HC/LC) | Residue | Site # | Site # | Site # |
| HC | T | 117 | 114 | 110 |
| HC | A | 143 | 140 | 136 |
| HC | L | 177 | 174 | 170 |
| HC | L | 182 | 179 | 175 |
| HC | T | 190 | 187 | 183 |
| HC | T | 212 | 209 | 205 |
| HC | V | 265 | 262 | 258 |
| HC | G | 374 | 371 | 367 |
| HC | Y | 376 | 373 | 369 |
| HC | E | 385 | 382 | 378 |
| HC | S | 427 | 424 | 420 |
| HC | N | 437 | 434 | 430 |
| HC | Q | 441 | 438 | 434 |

In some other embodiments, a THIOMAB™ antibody comprises one of the light chain cysteine substitutions listed in Table 6.

TABLE 6

| | | Screening | GNE | Kabat |
|---|---|---|---|---|
| Chain | | Mutation | Mutation | Mutation |
| (HC/LC) | Residue | Site # | Site # | Site # |
| LC | I | 106 | 106 | 106 |
| LC | R | 108 | 108 | 108 |
| LC | R | 142 | 142 | 142 |
| LC | K | 149 | 149 | 149 |

In some other embodiments, a THIOMAB™ antibody comprises one of the heavy or light chain cysteine substitutions listed in Table 7.

TABLE 7

| | | Screening | GNE | Kabat |
|---|---|---|---|---|
| Chain | | Mutation | Mutation | Mutation |
| (HC/LC) | Residue | Site # | Site # | Site # |
| LC | K | 149 | 149 | 149 |
| HC | A | 143 | 140 | 136 |
| HC | A | 121 | 118 | 114 |

Cysteine engineered antibodies which may be useful in the ADCs described herein for the treatment of cancer include, but are not limited to, antibodies against cell surface receptors and tumor-associated antigens (TAA). Tumor-associated antigens are known in the art, and can be prepared for use in generating antibodies using methods and information which are well known in the art. In attempts to discover effective cellular targets for cancer diagnosis and therapy, researchers have sought to identify transmembrane or otherwise tumor-associated polypeptides that are specifically expressed on the surface of one or more particular type(s) of cancer cell as compared to on one or more normal non-cancerous cell(s). Often, such tumor-associated polypeptides are more abundantly expressed on the surface of the cancer cells as compared to on the surface of the non-cancerous cells. The identification of such tumor-associated cell surface antigen polypeptides has given rise to the ability to specifically target cancer cells for destruction via antibody-based therapies.

In certain embodiments, an antibody provided herein may be further modified to contain additional nonproteinogenic moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, an antibody provided herein has a dissociation constant (K_{d}) of ≤1 μM, ≤100 nM, ≤50 nM, ≤10 nM, ≤5 nM, ≤1 nM, ≤0.1 nM, ≤0.01 nM, or ≤0.001 nM, and optionally is ≥10⁻¹³ M. (e.g. 10⁻⁸ M or less, e.g. from 10⁻⁸ M to 10⁻¹³ M, e.g., from 10⁻⁹ M to 10⁻¹³ M).

In one embodiment, K_{d} is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{d} is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

Antibody Fragments. In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). Fora review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

Chimeric and Humanized Antibodies. In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Human Antibodies. In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Library-Derived Antibodies. Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered human antibodies or human antibody fragments herein.

Multispecific Antibodies. In certain embodiments, an antibody provided herein is a multispecific antibody, e.g. a bispecific antibody. Multispecific antibodies are monoclonal antibodies that have binding specificities for at least two different sites. In certain embodiments, bispecific antibodies may bind to two different epitopes of the same target. Bispecific antibodies may also be used to localize cytotoxic agents to cells which express the target. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). The term “knob-into-hole” or “KnH” technology as used herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, CL:CH1 interfaces or VH/VL interfaces of antibodies (see, e.g., US 2011/0287009, US2007/0178552, WO 96/027011, WO 98/050431, Zhu et al., 1997, Protein Science 6:781-788, and WO2012/106587). In some embodiments, KnHs drive the pairing of two different heavy chains together during the manufacture of multispecific antibodies. For example, multispecific antibodies having KnH in their Fc regions can further comprise single variable domains linked to each Fc region, or further comprise different heavy chain variable domains that pair with similar or different light chain variable domains. KnH technology can be also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation.

The term “hole mutation” as used herein refers to a mutation that introduces a cavity (hole) into a polypeptide at an interface in which the polypeptide interacts with another polypeptide. In some embodiments, the other polypeptide has a knob mutation.

A “protuberance” refers to at least one amino acid side chain which projects from the interface of a first polypeptide and is therefore positionable in a compensatory cavity in the adjacent interface (i.e. the interface of a second polypeptide) so as to stabilize the heteromultimer, and thereby favor heteromultimer formation over homomultimer formation, for example. The protuberance may exist in the original interface or may be introduced synthetically (e.g., by altering nucleic acid encoding the interface). In some embodiments, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The side chain volumes of the various amino residues are shown, for example, in Table 1 of US2011/0287009. A mutation to introduce a “protuberance” may be referred to as a “knob mutation.”

In some embodiments, import residues for the formation of a protuberance are naturally occurring amino acid residues selected from arginine (R), phenylalanine (F), tyrosine (Y) and tryptophan (W). In some embodiments, an import residue is tryptophan or tyrosine. In some embodiment, the original residue for the formation of the protuberance has a small side chain volume, such as alanine, asparagine, aspartic acid, glycine, serine, threonine or valine.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g. by altering nucleic acid encoding the interface). In some embodiments, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. In some embodiments, import residues for the formation of a cavity are naturally occurring amino acid residues selected from alanine (A), serine (S), threonine (T) and valine (V). In some embodiments, an import residue is serine, alanine or threonine. In some embodiments, the original residue for the formation of the cavity has a large side chain volume, such as tyrosine, arginine, phenylalanine or tryptophan. A mutation to introduce a “cavity” may be referred to as a “hole mutation.”

The protuberance is “positionable” in the cavity which means that the spatial location of the protuberance and cavity on the interface of a first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe and Trp do not typically extend perpendicularly from the axis of the interface and have preferred conformations, the alignment of a protuberance with a corresponding cavity may, in some instances, rely on modeling the protuberance/cavity pair based upon a three-dimensional structure such as that obtained by X-ray crystallography or nuclear magnetic resonance (NMR). This can be achieved using widely accepted techniques in the art.

In some embodiments, a knob mutation in an IgG1 constant region is T366W (EU numbering). In some embodiments, a hole mutation in an IgG1 constant region comprises one or more mutations selected from T366S, L368A and Y407V (EU numbering). In some embodiments, a hole mutation in an IgG1 constant region comprises T366S, L368A and Y407V (EU numbering).

In some embodiments, a knob mutation in an IgG4 constant region is T366W (EU numbering). In some embodiments, a hole mutation in an IgG4 constant region comprises one or more mutations selected from T366S, L368A, and Y407V (EU numbering). In some embodiments, a hole mutation in an IgG4 constant region comprises T366S, L368A, and Y407V (EU numbering).

Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to the target as well as another, different antigen (see, US 2008/0069820, for example).

Antibody Variants. In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Substitution, Insertion, and Deletion Variants. In certain embodiments, antibody variants having one or more amino acid substitutions are provided. Sites of interest for substitutional mutagenesis include the HVRs and FRs. Conservative substitutions are shown in Table 8 under the heading of “preferred substitutions.” More substantial changes are provided in Table 8 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 8

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

Glycosylation variants. In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

Fc region variants. In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc (RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

In some embodiments, one or more amino acid modifications may be introduced into the Fc portion of the antibody provided herein in order to increase IgG binding to the neonatal Fc receptor. In certain embodiments, the antibody does not comprise the following three mutations according to EU numbering: M252Y, S254T, and T256E (the “YTE mutation”) (U.S. Pat. No. 8,697,650; see also Dall'Acqua et al., Journal of Biological Chemistry 281(33):23514-23524 (2006).

In certain embodiments, the YTE mutant provides a means to modulate antibody-dependent cell-mediated cytotoxicity (ADCC) activity of the antibody. In certain embodiments, the YTEO mutant provides a means to modulate ADCC activity of a humanized IgG antibody directed against a human antigen. See, e.g., U.S. Pat. No. 8,697,650; see also Dall'Acqua et al., Journal of Biological Chemistry 281(33):23514-23524 (2006).

In certain embodiments, the YTE mutant allows the simultaneous modulation of serum half-life, tissue distribution, and antibody activity (e.g., the ADCC activity of an IgG antibody). See, e.g., U.S. Pat. No. 8,697,650; see also Dall'Acqua et al., Journal of Biological Chemistry 281(33):23514-23524 (2006).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain embodiments, the proline at position 329 (EU numbering) (P329) of a wild-type human Fc region is substituted with glycine or arginine or an amino acid residue large enough to destroy the proline sandwich within the Fc/Fc gamma receptor interface, that is formed between the P329 of the Fc and tryptophane residues W87 and W110 of FcgRIII (Sondermann et al.: Nature 406, 267-273 (20 Jul. 2000)). In a further embodiment, at least one further amino acid substitution in the Fc variant is S228P, E233P, L234A, L235A, L235E, N297A, N297D, or P331S and still in another embodiment said at least one further amino acid substitution is L234A and L235A of the human IgG1 Fc region or S228P and L235E of the human IgG4 Fc region, all according to EU numbering (U.S. Pat. No. 8,969,526 which is incorporated by reference in its entirety).

In certain embodiments, a polypeptide comprises the Fc variant of a wild-type human IgG Fc region wherein the polypeptide has P329 of the human IgG Fc region substituted with glycine and wherein the Fc variant comprises at least two further amino acid substitutions at L234A and L235A of the human IgG1 Fc region or S228P and L235E of the human IgG4 Fc region, and wherein the residues are numbered according to the EU numbering (U.S. Pat. No. 8,969,526 which is incorporated by reference in its entirety). In certain embodiments, the polypeptide comprising the P329G, L234A and L235A (EU numbering) substitutions exhibit a reduced affinity to the human FcγRIIIA and FcγRIIA, for down-modulation of ADCC to at least 20% of the ADCC induced by the polypeptide comprising the wildtype human IgG Fc region, and/or for down-modulation of ADCP (U.S. Pat. No. 8,969,526 which is incorporated by reference in its entirety).

In a specific embodiment the polypeptide comprising an Fc variant of a wildtype human Fc polypeptide comprises a triple mutation: an amino acid substitution at position Pro329, a L234A and a L235A mutation according to EU numbering (P329/LALA) (U.S. Pat. No. 8,969,526 which is incorporated by reference in its entirety). In specific embodiments, the polypeptide comprises the following amino acid substitutions: P329G, L234A, and L235A according to EU numbering.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

In one embodiment, the ADC comprises BPA peptide (e.g. BPA7 or BPA10) and an antibody described herein. In one embodiment, the ADC comprises BPA peptide (e.g. BPA7 or BPA10), an extension moiety comprising SATA-PEG₍₂₋₁₂₎, and an antibody described herein. In one embodiment, the ADC comprises BPA7 or BPA10, an antibody described herein, and D covalently attached to the BPA peptide via L having formula (IV). In one preferred embodiment, the ADC comprises BPA7 or BPA10, an extension moiety comprising SATA-PEG₍₂₋₁₂₎, an antibody described herein, and D as described herein covalently attached to the BPA peptide extension moiety via L having formula (IV) as described herein.

In one embodiment, the ADC comprises BPA peptide (e.g. BPA7 or BPA10) and trastuzumab. In one embodiment, the ADC comprises BPA peptide (e.g. BPA7 or BPA10), an extension moiety comprising SATA-PEG₍₂₋₁₂₎, and trastuzumab. In one embodiment, the ADC comprises BPA7 and trastuzumab. In one embodiment, the ADC comprises BPA7, an extension moiety comprising SATA-PEG₍₂₋₁₂₎, and trastuzumab. In one embodiment, the ADC comprises BPA7 or BPA10, trastuzumab, and D covalently attached to the BPA peptide via L having formula (IV). In one preferred embodiment, the ADC comprises BPA7 or BPA10, an extension moiety comprising SATA-PEG₍₂₋₁₂₎, trastuzumab, and D as described herein covalently attached to the BPA peptide extension moiety via L having formula (IV) as described herein.

Further provided herein are ADCs comprising two or more different drug moieties. In one embodiment, the ADCs provided herein comprise a second drug (D2) covalently attached to another residue in the antibody (e.g. a cysteine of a THIOMAB™). Thus, also provided herein are ADC compositions and methods of synthesizing ADC compositions comprising conjugation of different drug moieties to the same antibody. For example, an ADC described herein can comprise an antibody such as trastuzumab conjugated to a second drug moiety such as emtansine thereby forming an ADC (e.g. KADCYLA) wherein that ADC is further conjugated to BPA peptide and a second drug (D) as described herein.

Recombinant Methods and Compositions. Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977); baby hamster kidney cells (BHK); mouse sertoli cells (TM4 cells as described, e.g., in Mather, Biol. Reprod. 23:243-251 (1980); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Pharmaceutical formulations of therapeutic antibody-drug conjugates (ADC) of the invention are typically prepared for parenteral administration, i.e. bolus, intravenous, intratumor injection with a pharmaceutically acceptable parenteral vehicle and in a unit dosage injectable form. An antibody-drug conjugate (ADC) having the desired degree of purity is optionally mixed with one or more pharmaceutically acceptable excipients or stabilizers (Remington's Pharmaceutical Sciences (1980) 16th edition, Osol, A. Ed.), in the form of a lyophilized formulation or an aqueous solution. Such excipients include pharmaceutically acceptable salts, buffers, and other stabilizing agents known in the art.

The antibody-drug conjugates (ADC) of the invention may be administered by any route appropriate to the condition to be treated. The ADC will typically be administered parenterally, i.e. infusion, subcutaneous, intramuscular, intravenous, intradermal, intrathecal and epidural.

In another embodiment of the invention, an article of manufacture, or “kit”, containing materials useful for the treatment of the disorders described above is provided. The article of manufacture comprises a container and a label or package insert on or associated with the container. Suitable containers include, for example, bottles, vials, syringes, blister pack, etc. The containers may be formed from a variety of materials such as glass or plastic. The container holds an antibody-drug conjugate (ADC) composition which is effective for treating the condition and may have a sterile access port (for example the container may be an intravenous solution bag or a vial having a stopper pierceable by a hypodermic injection needle). At least one active agent in the composition is an ADC. The label or package insert indicates that the composition is used for treating the condition of choice, such as cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Methods of synthesizing the ADCs described herein are provided herein. In one embodiment is a method to prepare an antibody-drug conjugate as described herein where the method comprises:
(i) reacting an antibody under photo-crosslinking conditions with a BPA peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11 thereby forming an antibody conjugate;
(ii) optionally removing a protecting group on the terminal end of the BPA peptide; and
(iii) reacting the antibody conjugate with a drug (D) to form the antibody-drug conjugate composition having Formula (I).

Further provided herein is a method to prepare an antibody-drug conjugate composition as described herein where the method comprises reacting an antibody under photo-crosslinking conditions with a BPA peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11, wherein the BPA peptide is covalently attached to a drug moiety (D) described herein thereby forming the antibody conjugate

In one embodiment of the methods above, the BPA peptide comprises an extension moiety as described herein. In one embodiment of the methods above, where the BPA peptide comprises an extension moiety, the extension moiety comprises SATA-PEG₍₂₋₁₂₎ as described herein. In one embodiment of the methods above, D further comprises a linker, wherein the linker as described herein. In one embodiment of the methods above, the linker comprises formula (IV):

-Str-(Pep)ₘ-(Y)ₙ—

where

Str is a stretcher unit or S covalently attached the BPA peptide;

Pep is an optional peptide unit of two to twelve amino acid residues;

Y is an optional spacer unit covalently attached to D; and

m and n are independently selected from 0 and 1.

In one preferred embodiment of the methods above, the BPA peptide is BPA7 as described herein. In one embodiment of the methods above, the BPA peptide is BPA1 or BPA2. In one embodiment of the methods above, the BPA peptide is BPA4. In another preferred embodiment, the BPA peptide is BPA10.

In one embodiment, the antibody is a monoclonal IgG antibody as described herein. In one embodiment, the antibody is a cysteine-engineered antibody (e.g. a THIOMAB™) as described herein. In one preferred embodiment, the antibody is a HER2 specific antibody (e.g. trastuzumab). In one preferred embodiment, the antibody is a therapeutic antibody as set forth herein.

In one embodiment of the methods above, D is an anticancer moiety as described herein.

In one embodiment of the methods above, the photo-crosslinking conditions comprise irradiating under ultraviolet (UV) light. In one embodiment of the methods above, the photo-crosslinking conditions comprise irradiating under ultraviolet (UV) light the antibody and the BPA peptide in a multi-well plate. In one embodiment of the methods above, the antibody and the BPA peptide are irradiated with 365 nm UV light. In one embodiment of the methods above, the photo-crosslinking conditions further comprise an antioxidant. In one embodiment of the methods above, the antioxidant is selected from the group consisting of 5-hydroxyindole (5-HI), methionine, sodium thiosulfate, catalase, platinum, tryptophan, 5-methoxy-tryptophan, 5-amino-tryptophan, 5-fluoro-tryptophan, N-acetyl tryptophan, tryptamine, tryptophanamide, serotonin, melatonin, kynurenine, indole derivatives (indole, indole-3-acetic acid, 4-hydroxy indole, 5-hydroxy indole, 5-hydroxy indole 3-acetic acid, 7-hydroxy indole, 7-hydroxy indole 2-carboxylic acid), salicylic acid, 5-hydroxy salicylic acid, anthranilic acid, and 5-hydroxy anthranilic acid. In one embodiment, the antioxidant is 5-hydroxyindole.

In one example, the BPA peptides of Table 1, can be prepared as N-terminal acetyl and C-terminal amides, and photocrosslinked with antibody fragment such as trastuzumab Fc (HERCEPTIN®, Genentech) under the conditions described within the example provided herein.

In one embodiment BPA peptide BPA7 can be photocrosslinked as described herein with an antibody described herein. In one example, BPA peptide BPA7 can be photocrosslinked under different photo-crosslinking conditions with an IgG antibody, such as, for example, trastuzumab or rituximab. The duration, temperature, proximity to UV light source, buffer composition and pH, and addition or concentration of an anti-oxidant, such as 5-HI, can be varied. Reactions can be performed in clear 96-well plates, uncovered with 150 micro liter (μL) final volume. Photocrosslinking of a BPA peptide described herein with an antibody described herein can be measured by techniques known in the art. For example, photocrosslinking can be measured by mass spectrometric quantitation of fragments after digestion (e.g. with IdeS) of the product to generate the Fab′2 and Fc/2 cleavage products. For example, photocrosslinking of BPA7 peptide with trastuzumab was measured by mass spectrometric quantitation of fragments after digestion of the product with IdeS to generate the Fab′2 and Fc/2 cleavage products. The presence and absence of the BPA peptide covalently attached to the antibody fragments were detected as a shift in molecular mass corresponding to the mass of the BPA peptide.

In other embodiments, a BPA peptide (e.g. BPA7) can be photocrosslinked to a cysteine-engineered antibody as described herein. FIG. 7 shows graphically photocrosslinking of a cyclic disulfide BPA peptide to the Fc region of a cysteine-engineered antibody (THIOMAB®, Genentech, Inc.) where the cysteine thiol is denoted by a star attached in the light chains of the antibody. Free cysteine thiol groups remaining after the photo-crosslinkingphoto-crosslinking conditions can be reacted with a cysteine-reactive moiety (demonstrated by reaction with 1-ethyl-1H-pyrrole-2,5-dione (EMCA)). The photocrosslinked peptide to antibody ratio (PAR) was measured by mass spectrometry before and after photocrosslinking as described herein.

Also provided herein are conjugates comprising a BPA peptide described herein (e.g. BPA7) and an IgG4 or IgG1 subclass of IgG antibody. In one embodiment, different subclasses of IgG antibodies can be photocrosslinked with BPA peptide BPA7 or variants thereof. In one embodiment, the BPA peptide includes a mutation where the valine residue of Fc-III is replaced with a BPA residue.

A “linker drug reagent” as used herein refers to a reagent comprising a D described herein together with a L as described herein.

In one embodiment, photoconjugation methods described herein allow for the generation of homogeneous antibody conjugates. In one embodiment, the photoconjugation methods and antibodies described herein increase ADC half-life. In one embodiment, the photoconjugation methods and antibodies described herein increase ADC half-life.

In one embodiment, the antibodies and methods of making antibody conjugates described herein are useful for radioactivity-based immunotherapy or imaging. In one embodiment, an antibody described herein is conjugated to a radiolabel (e.g. ¹¹C, ¹³N, ¹⁵O, ¹⁸F, ³²P, ⁵¹Cr, ⁵⁷Co, ⁶⁴Cu, ⁶⁷Ga, ⁷⁵Se, ^{81m}Kr, ⁸²Rb, ^{99m}Tc, ¹²³I, ¹²⁵I, ¹³¹I, ¹¹¹In, and ²⁰¹Ti) making an ADC thereof. In one embodiment, such antibody conjugates enhance image contrast or reduce radiation-induced toxicity.

In another embodiment, the antibodies and methods described are useful as ocular antibody conjugate therapeutics. In one embodiment, the antibodies and methods described herein mediate or direct antibody conjugate therapeutics to a particular location in the eye (e.g. retina) and/or bind to biologically-active molecules in the eye (e.g., VEGF).

In one embodiment, the methods described herein are used to generate libraries of homogeneously-labeled antibody conjugates from hybridomas provided a host species that produces antibodies comprising a Met-252 residue in the Fc domain. In one embodiment of such methods, the methods use a multi-well plate (e.g. a 96-well plate). In one embodiment of such methods, the antibody amount is about 0.3 mg, 0.4 mg, 0.5 mg, 0.6 mg, or 0.7 mg, including values therein.

In another embodiment, the ADCs described herein are useful in treatment of cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Provided herein are methods for treating or delaying the progression of cancer in a patient having cancer by administering to the patient an effective amount of an ADC described herein and a taxane (e.g., nab-paclitaxel (ABRAXANE®) or paclitaxel). In some embodiments, the treatment results in a response in the individual after treatment with an ADC as described herein. In some embodiments, the response is a complete response (CR). In one embodiment, the response is a partial response (PR). In some embodiments, the treatment results in a sustained response in the individual after cessation of the treatment. The methods described herein further include treating conditions where enhanced immunogenicity is desired such as increasing tumor immunogenicity for the treatment of cancer. In some embodiments, the methods further comprise administering a platinum-based chemotherapeutic agent. In some embodiments, the platinum-based chemotherapeutic agent is carboplatin.

In some embodiments, the cancer is breast cancer as described herein, bladder cancer (e.g., UBC, MIBC, and NMIBC) as described herein, colorectal cancer, rectal cancer, lung cancer (e.g., non-small cell lung cancer that can be squamous or non-squamous) as described herein, glioblastoma, non-Hodgkins lymphoma (NHL), renal cell cancer (e.g., RCC), prostate cancer, liver cancer, pancreatic cancer, soft-tissue sarcoma, kaposi's sarcoma, carcinoid carcinoma, head and neck cancer, gastric cancer, esophageal cancer, prostate cancer, endometrial cancer, kidney cancer, ovarian cancer, mesothelioma, and heme malignancies (e.g., MDS and multiple myeloma).

In some embodiments, the cancer is selected from: small cell lung cancer, glioblastoma, neuroblastomas, melanoma, gastric cancer, colorectal cancer (CRC), or hepatocellular carcinoma. In particular embodiments, the cancer is selected from lung cancer (e.g., non-small cell lung cancer that can be squamous or non-squamous, bladder cancer (e.g., UBC), breast cancer (e.g., TNBC), RCC, melanoma, or breast cancer. In another embodiment, the cancer is a heme malignancy (e.g., MDS and multiple myeloma).

In some embodiments, the lung cancer is non-small cell lung cancer that can be squamous or non-squamous. In some embodiments, the bladder cancer is UBC. In some embodiments, the breast cancer is TNBC. In some embodiments, the heme malignancy is a MDS or multiple myeloma.

In certain instances, the cancer may be a lung cancer. For example, the lung cancer may be a non-small cell lung cancer (NSCLC), including but not limited to a locally advanced or metastatic (e.g., stage IIIB, stage IV, or recurrent) NSCLC. In some instances, the lung cancer (e.g., NSCLC) is unresectable/inoperable lung cancer (e.g., NSCLC). The methods described herein may be used for treating a patient having a lung cancer described herein who may benefit from treatment including an ADC described herein.

In certain instances, the cancer may be a bladder cancer. For example, the bladder cancer may be a urothelial bladder cancer, including but not limited to a non-muscle invasive urothelial bladder cancer, a muscle-invasive urothelial bladder cancer, or a metastatic urothelial bladder cancer. In some instances, the urothelial bladder cancer is a metastatic urothelial bladder cancer. The methods described herein may be used for treating a patient having a bladder cancer (e.g., UBC) who may benefit from treatment including an ADC described herein.

In certain instances, the cancer may be a kidney cancer. In some instances, the kidney cancer may be a renal cell carcinoma (RCC), including stage I RCC, stage II RCC, stage III RCC, stage IV RCC, or recurrent RCC. The methods described herein may be used for treating a patient having a kidney cancer (e.g., RCC) who may benefit from treatment including an ADC described herein.

In certain instances, the cancer may be a breast cancer. For example, the breast cancer may be TNBC, estrogen receptor-positive breast cancer, estrogen receptor-positive/HER2-negative breast cancer, HER2-negative breast cancer, HER2-positive breast cancer, estrogen receptor-negative breast cancer, progesterone receptor-positive breast cancer, or progesterone receptor-negative breast cancer. The methods described herein may be used for treating a patient having a breast cancer as described herein who may benefit from treatment including an ADC described herein.

In some embodiments, the patient has been treated with a cancer therapy before the combination treatment with an ADC described herein. In some embodiments, the patient has cancer that is resistant to one or more cancer therapies. In some embodiments, resistance to cancer therapy includes recurrence of cancer or refractory cancer. Recurrence may refer to the reappearance of cancer, in the original site or a new site, after treatment. In some embodiments, resistance to a cancer therapy includes progression of the cancer during treatment with the anti-cancer therapy. In some embodiments, resistance to a cancer therapy includes cancer that does not response to treatment. The cancer may be resistant at the beginning of treatment or it may become resistant during treatment. In some embodiments, the cancer is at early stage or at late stage.

In some embodiments, the ADCs described herein can be combined with other anticancer therapies providing for a combination therapy thereof. An ADC described herein and the second anticancer therapy may be administered by the same route of administration or by different routes of administration. In some embodiments, an ADC described herein is administered intravenously, intramuscularly, subcutaneously, topically, orally, transdermally, intraperitoneally, intraorbitally, by implantation, by inhalation, intrathecally, intraventricularly, or intranasally. In some embodiments, the taxane is administered intravenously, intramuscularly, subcutaneously, topically, orally, transdermally, intraperitoneally, intraorbitally, by implantation, by inhalation, intrathecally, intraventricularly, or intranasally. The appropriate dosage of an ADC described herein may be determined based on the type of disease to be treated, the type of an ADC described herein and the second anticancer therapy, the severity and course of the disease, the clinical condition of the patient, the patient's clinical history and response to the treatment, and the discretion of the attending physician.

As a general proposition, the therapeutically effective amount of an ADC described herein administered to a patient provided herein will be in the range of about 0.01 to about 50 mg/kg of patient body weight whether by one or more administrations. In some embodiments, the antibody used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the antibody is administered at 15 mg/kg. However, other dosage regimens may be useful. In one embodiment, an ADC described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg on day 1 of 21-day cycles. In some embodiments, an ADC is administered to a patient described herein in an amount as set forth above in combination with an anti-PD-L1 antibody (e.g. atezolizumab). Atezolizumab can be administered in accordance with a package insert or alternatively, can be administered at 1200 mg IV every three weeks (q3w). The dose may be administered as a single dose or as multiple doses (e.g., 2 or 3 doses), such as infusions. The dose of the ADC administered in a combination treatment may be reduced as compared to a single treatment. The progress of this therapy is easily monitored by conventional techniques. In one embodiment, an ADC described herein is administered in the form of adjuvant or neoadjuvant therapy.

In some embodiments, the methods provided herein may further comprise an additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. The additional therapy may be one or more of the chemotherapeutic agents described herein.

In one embodiment, is a method of treating breast cancer where the method comprises administering to a patient having breast cancer, an effective amount of an ADC described herein. The breast cancer can be early breast cancer or non-metastatic breast cancer. The breast cancer can be advanced breast cancer or metastatic breast cancer. In one embodiment is a method for treating hormone receptor positive (HR+) breast cancer (also called estrogen receptor positive (ER+) breast cancer or estrogen receptor positive and/or progesterone receptor positive (PR+) breast cancer), by administering an effective amount of ADC described herein. In another embodiment, the breast cancer is early or locally advanced hormone receptor positive (HR+) breast cancer, also named early or locally advanced ER+ breast cancer. In still another embodiment, the breast cancer is advanced hormone receptor positive (HR+) breast cancer or metastatic hormone receptor positive (HR+) breast cancer, also named advanced ER+ breast cancer or metastatic ER+ breast cancer.

Standard of care for breast cancer is determined by both disease (tumor, stage, pace of disease, etc.) and patient characteristics (age, by biomarker expression and intrinsic phenotype). General guidance on treatment options are described in the NCCN Guidelines (e.g., NCCN Clinical Practice Guidelines in Oncology, Breast Cancer, version 2.2016, National Comprehensive Cancer Network, 2016, pp. 1-202), and in the ESMO Guidelines (e.g., Senkus, E., et al. Primary Breast Cancer: ESMO Clinical Practice Guidelines for diagnosis, treatment and follow-up. Annals of Oncology 2015; 26(Suppl. 5): v8-v30; and Cardoso F., et al. Locally recurrent or metastatic breast cancer: ESMO Clinical Practice Guidelines for diagnosis, treatment and follow-up. Annals of Oncology 2012; 23 (Suppl. 7):vii11-vii19.).

ADCs described herein can be used either alone or in combination with standard of care treatment options for breast cancer, which in general include surgery, systemic chemotherapy (either pre- or post-operatively) and/or radiation therapy. Depending on tumor and patient characteristics, systemic chemotherapy may be administered as adjuvant (post-operative) therapy or as neoadjuvant (pre-operative) therapy.

In one embodiment is a method of treating a cancer described herein (e.g. breast cancer) by administering an ADC described herein in combination with one or more therapeutic antibodies as provided herein.

In some embodiments, an ADC described herein is administered in conjunction with an agonist directed against an activating co-stimulatory molecule. In some embodiments, an activating co-stimulatory molecule may include CD40, CD226, CD28, OX40, GITR, CD137, CD27, HVEM, or CD127. In some embodiments, the agonist directed against an activating co-stimulatory molecule is an agonist antibody that binds to CD40, CD226, CD28, OX40, GITR, CD137, CD27, HVEM, or CD127. In some embodiments, an ADC described herein is administered in conjunction with an antagonist directed against an inhibitory co-stimulatory molecule. In some embodiments, an inhibitory co-stimulatory molecule includes CTLA-4 (also known as CD152), PD-1, TIM-3, BTLA, VISTA, LAG-3, B7-H3, B7-H4, IDO, TIGIT, MICA/B, or arginase. In some embodiments, the antagonist directed against an inhibitory co-stimulatory molecule is an antagonist antibody that binds to CTLA-4, PD-1, TIM-3, BTLA, VISTA, LAG-3, B7-H3, B7-H4, IDO, TIGIT, MICA/B, or arginase.

In some embodiments, an ADC described herein is administered in conjunction with an antagonist directed against CTLA-4 (also known as CD152), for example, a blocking antibody. In some embodiments, an ADC described herein is administered in conjunction with ipilimumab (also known as MDX-010, MDX-101, or YERVOY®). In some embodiments, an ADC described herein is administered in conjunction with tremelimumab (also known as ticilimumab or CP-675,206). In some embodiments, an ADC described herein is administered in conjunction with an antagonist directed against B7-H3 (also known as CD276), for example, a blocking antibody. In some embodiments, an ADC described herein is administered in conjunction with MGA271. In some embodiments, an ADC described herein is administered in conjunction with an antagonist directed against a TGF beta, for example, metelimumab (also known as CAT-192), fresolimumab (also known as GC1008), or LY2157299.

In some embodiments, an ADC described herein is administered in conjunction with a treatment comprising adoptive transfer of a T cell (e.g., a cytotoxic T cell or CTL) expressing a chimeric antigen receptor (CAR). In some embodiments, an ADC described herein is administered in conjunction with a treatment comprising adoptive transfer of a T cell comprising a dominant-negative TGF beta receptor, e.g., a dominant-negative TGF beta type II receptor. In some embodiments, an ADC described herein is administered in conjunction with a treatment comprising a HERCREEM protocol (see, e.g., ClinicalTrials.gov Identifier NCT00889954).

In some embodiments, an ADC described herein is administered in conjunction with an agonist directed against CD137 (also known as TNFRSF9, 4-1BB, or ILA), for example, an activating antibody. In some embodiments, an ADC described herein is administered in conjunction with urelumab (also known as BMS-663513). In some embodiments, an ADC described herein is administered in conjunction with an agonist directed against CD40, for example, an activating antibody. In some embodiments, an ADC described herein is administered in conjunction with CP-870893. In some embodiments, an ADC described herein is administered in conjunction with an agonist directed against OX40 (also known as CD134), for example, an activating antibody. In some embodiments, an ADC described herein is administered in conjunction with an anti-OX40 antibody (e.g., AgonOX). In some embodiments, an ADC described herein is administered in conjunction with an agonist directed against CD27, for example, an activating antibody. In some embodiments, an ADC described herein is administered in conjunction with CDX-1127. In some embodiments, an ADC described herein is administered in conjunction with an antagonist directed against indoleamine-2,3-dioxygenase (IDO). In some embodiments, with the IDO antagonist is 1-methyl-D-tryptophan (also known as 1-D-MT).

In some embodiments, an ADC described herein is administered in conjunction with an antibody-drug conjugate. In some embodiments, the antibody-drug conjugate comprises mertansine or monomethyl auristatin E (MMAE). In some embodiments, an ADC described herein is administered in conjunction with and anti-NaPi2b antibody-MMAE conjugate (also known as DNIB0600A or RG7599). In some embodiments, an ADC described herein is administered in conjunction with trastuzumab emtansine (also known as T-DM1, ado-trastuzumab emtansine, or KADCYLA®, Genentech). In some embodiments, an ADC described herein is administered in conjunction with DMUC5754A. In some embodiments, an ADC described herein is administered in conjunction with an antibody-drug conjugate targeting the endothelin B receptor (EDNBR), for example, an antibody directed against EDNBR conjugated with MMAE.

In some embodiments, an ADC described herein is administered in conjunction with an angiogenesis inhibitor. In some embodiments, an ADC described herein is administered in conjunction with an antibody directed against a VEGF, for example, VEGF-A. In some embodiments, an ADC described herein is administered in conjunction with bevacizumab (also known as AVASTIN®, Genentech). In some embodiments, an ADC described herein is administered in conjunction with an antibody directed against angiopoietin 2 (also known as Ang2). In some embodiments, an ADC described herein is administered in conjunction with MED13617.

In some embodiments, an ADC described herein is administered in conjunction with an antineoplastic agent. In some embodiments, an ADC described herein is administered in conjunction with an agent targeting CSF-1R (also known as M-CSFR or CD115). In some embodiments, an ADC described herein is administered in conjunction with anti-CSF-1R (also known as IMC-CS4). In some embodiments, an ADC described herein is administered in conjunction with an interferon, for example interferon alpha or interferon gamma. In some embodiments, an ADC described herein is administered in conjunction with Roferon-A (also known as recombinant Interferon alpha-2a). In some embodiments, an ADC described herein is administered in conjunction with GM-CSF (also known as recombinant human granulocyte macrophage colony stimulating factor, rhu GM-CSF, sargramostim, or LEUKINE®). In some embodiments, an ADC described herein is administered in conjunction with IL-2 (also known as aldesleukin or PROLEUKIN®). In some embodiments, an ADC described herein is administered in conjunction with IL-12. In some embodiments, an ADC described herein is administered in conjunction with an antibody targeting CD20. In some embodiments, the antibody targeting CD20 is obinutuzumab (also known as GA101 or GAZYVA®) or rituximab. In some embodiments, an ADC described herein is administered in conjunction with an antibody targeting GITR. In some embodiments, the antibody targeting GITR is TRX518.

In some embodiments, an ADC described herein is administered in conjunction with a cancer vaccine. In some embodiments, the cancer vaccine is a peptide cancer vaccine, which in some embodiments is a personalized peptide vaccine. In some embodiments the peptide cancer vaccine is a multivalent long peptide, a multi-peptide, a peptide cocktail, a hybrid peptide, or a peptide-pulsed dendritic cell vaccine (see, e.g., Yamada et al., Cancer Sci, 104:14-21, 2013). In some embodiments, an ADC described herein is administered in conjunction with an adjuvant. In some embodiments, an ADC described herein is administered in conjunction with a treatment comprising a TLR agonist, for example, Poly-ICLC (also known as HILTONOL®), LPS, MPL, or CpG ODN. In some embodiments, an ADC described herein is administered in conjunction with tumor necrosis factor (TNF) alpha. In some embodiments, an ADC described herein is administered in conjunction with IL-1. In some embodiments, an ADC described herein is administered in conjunction with HMGB1. In some embodiments, an ADC described herein is administered in conjunction with an IL-10 antagonist. In some embodiments, an ADC described herein is administered in conjunction with an IL-4 antagonist. In some embodiments, an ADC described herein is administered in conjunction with an IL-13 antagonist. In some embodiments, an ADC described herein is administered in conjunction with an HVEM antagonist. In some embodiments, an ADC described herein is administered in conjunction with an ICOS agonist, e.g., by administration of ICOS-L, or an agonistic antibody directed against ICOS. In some embodiments, an ADC described herein is administered in conjunction with a treatment targeting CX3CL1. In some embodiments, an ADC described herein is administered in conjunction with a treatment targeting CXCL9. In some embodiments, an ADC described herein is administered in conjunction with a treatment targeting CXCL10. In some embodiments, an ADC described herein is administered in conjunction with a treatment targeting CCL5. In some embodiments, an ADC described herein is administered in conjunction with an LFA-1 or ICAM1 agonist. In some embodiments, an ADC described herein is administered in conjunction with a Selectin agonist.

In some embodiments, an ADC described herein is administered in conjunction with a targeted therapy. In some embodiments, an ADC described herein is administered in conjunction with an inhibitor of B-Raf. In some embodiments, an ADC described herein is administered in conjunction with vemurafenib (also known as ZELBORAF®). In some embodiments, an ADC described herein is administered in conjunction with dabrafenib (also known as TAFINLAR®). In some embodiments, an ADC described herein is administered in conjunction with erlotinib (also known as TARCEVA®). In some embodiments, an ADC described herein is administered in conjunction with an inhibitor of a MEK, such as MEK1 (also known as MAP2K1) or MEK2 (also known as MAP2K2). In some embodiments, an ADC described herein is administered in conjunction with cobimetinib (also known as GDC-0973 or XL-518). In some embodiments, an ADC described herein is administered in conjunction with trametinib (also known as MEKINIST®). In some embodiments, an ADC described herein is administered in conjunction with an inhibitor of K-Ras. In some embodiments, an ADC described herein is administered in conjunction with an inhibitor of c-Met. In some embodiments, an ADC described herein is administered in conjunction with onartuzumab (also known as MetMAb). In some embodiments, an ADC described herein is administered in conjunction with an inhibitor of Alk. In some embodiments, an ADC described herein is administered in conjunction with AF802 (also known as CH5424802 or alectinib). In some embodiments, an ADC described herein is administered in conjunction with an inhibitor of a phosphatidylinositol 3-kinase (PI3K). In some embodiments, an ADC described herein is administered in conjunction with BKM120. In some embodiments, an ADC described herein is administered in conjunction with idelalisib (also known as GS-1101 or CAL-101). In some embodiments, an ADC described herein is administered in conjunction with perifosine (also known as KRX-0401). In some embodiments, an ADC described herein is administered in conjunction with an inhibitor of an Akt (e.g. GDC-0068 also known as ipatasertib). In some embodiments, an ADC described herein is administered in conjunction with MK2206. In some embodiments, an ADC described herein is administered in conjunction with GSK690693. In some embodiments, an ADC described herein is administered in conjunction with GDC-0941. In some embodiments, an ADC described herein is administered in conjunction with an inhibitor of mTOR. In some embodiments, an ADC described herein is administered in conjunction with sirolimus (also known as rapamycin). In some embodiments, an ADC described herein is administered in conjunction with temsirolimus (also known as CCI-779 or TORISEL®). In some embodiments, an ADC described herein is administered in conjunction with everolimus (also known as RAD001). In some embodiments, an ADC described herein is administered in conjunction with ridaforolimus (also known as AP-23573, MK-8669, or deforolimus). In some embodiments, an ADC described herein is administered in conjunction with OSI-027. In some embodiments, an ADC described herein is administered in conjunction with AZD8055. In some embodiments, an ADC described herein is administered in conjunction with INK128. In some embodiments, an ADC described herein is administered in conjunction with a dual PI3K/mTOR inhibitor. In some embodiments, an ADC described herein is administered in conjunction with XL765. In some embodiments, an ADC described herein is administered in conjunction with GDC-0980. In some embodiments, an ADC described herein is administered in conjunction with BEZ235 (also known as NVP-BEZ235). In some embodiments, an ADC described herein is administered in conjunction with BGT226. In some embodiments, an ADC described herein is administered in conjunction with GSK2126458. In some embodiments, an ADC described herein is administered in conjunction with PF-04691502. In some embodiments, an ADC described herein is administered in conjunction with PF-05212384 (also known as PKI-587).

In some embodiments, the ADCs described herein are for use in a combination therapy for the treatment of breast cancer in combination with one or more other therapeutic agents. Thus, in some embodiments herein are methods of treating breast cancer in a patient having breast cancer by administering an ADC described herein in combination with one or more other therapeutic agents. In one embodiment, the ADCs described herein are for use in a combination therapy for the treatment of early breast cancer or locally advanced breast cancer. In one embodiment, the ADCs described herein are for use in a combination therapy for the treatment of advanced breast cancer or metastatic breast cancer.

In one embodiment is a method of treating a breast cancer described herein in a patient having such a breast cancer by administering an effective amount of an ADC described herein and administering an effective amount of doxorubicin and cyclophosphamide (AC chemotherapy). In one embodiment is a method of treating a breast cancer described herein in a patient having such a breast cancer by administering an effective amount of an ADC described herein and administering an effective amount of docetaxel, doxorubicin and cyclophosphamide (TAC chemotherapy). In one embodiment is a method of treating a breast cancer described herein in a patient having such a breast cancer by administering an effective amount of an ADC described herein and administering an effective amount of cyclophosphamide, methotrexate and 5-fluorouracil (CMF chemotherapy). In one embodiment is a method of treating a breast cancer described herein in a patient having such a breast cancer by administering an effective amount of an ADC described herein and administering an effective amount of epirubicin and cyclophosphamide (EC chemotherapy). In one embodiment is a method of treating a breast cancer described herein in a patient having such a breast cancer by administering an effective amount of an ADC described herein and administering an effective amount of 5-fluorouracil, epirubicin and cyclophosphamide (FEC chemotherapy). In one embodiment is a method of treating a breast cancer described herein in a patient having such a breast cancer by administering an effective amount of an ADC described herein and administering an effective amount of 5-fluorouracil, doxorubicin and cyclophosphamide (FAC chemotherapy). In one embodiment is a method of treating a breast cancer described herein in a patient having such a breast cancer by administering an effective amount of an ADC described herein and administering an effective amount of taxane, in particular docetaxel or paclitaxel (including albumin-bound paclitaxel ABRAXANE).

In one embodiment, when ADCs described herein are used in the methods treatment of metastatic breast cancer as described herein, the methods of treating comprise administering to such a patient an effective amount of an ADC described herein and administering an effective amount of at least one additional therapeutic agent such as doxorubicin, pegylated liposomal doxorubicin, epirubicin, cyclophosphamide, carboplatin, cisplatin, docetaxel, paclitaxel, albumin-bound paclitaxel, capecitabine, gemcitabine, vinorelbine, eribulin, Ixabepilone, methotrexate, or 5-fluorouracil (5-FU). In one embodiment is a method of treating a breast cancer described herein in a patient having such breast cancer by administering an effective amount of an ADC described herein and administering an effective amount of docetaxel and capecitabine. In one embodiment is a method of treating a breast cancer described herein in a patient having such breast cancer by administering an effective amount of an ADC described herein and administering an effective amount of gemcitabine and paclitaxel.

In another embodiment, is a method of treating a breast cancer described herein in a patient having such a breast cancer by administering an effective amount of an ADC described herein in combination with chemotherapy and/or radiation therapy. In one embodiment is a method of treating ER+ breast cancer, the method comprising administering to a patient having ER+ breast cancer an effective amount of an ADC as described herein in combination with an effective amount of fulvestrant, palbociclib, anastrozole, letrozole, or exemestane. In one embodiment, is a method of treating Her2+ breast cancer the method comprising administering to a patient having ER+ breast cancer an effective amount of an ADC as described herein in combination with an effective amount of (1) pertuzumab; (2) trastuzumab and pertuzumab; or (3) trastuzumab and one or more chemotherapy agents comprising capecitabine, gemcitabine, carboplatin, cisplatin, cyclophosphamide, docetaxel, paclitaxel, doxorubicin, epirubicin, eribulin, 5-fluorouracil, Ixabepilone, liposomal doxorubicin, methotrexate, albumin bound paclitaxel, or vinorelbine.

In some embodiments is a method of treating hormone receptor positive (HR+) breast cancer or estrogen receptor positive (ER+) breast cancer by administering to a patient having such breast cancer an effective amount of an ADC described herein. In one embodiment, is a method of treating early or locally advanced hormone receptor positive (HR+) breast cancer, also named early or locally advanced ER+ breast cancer by administering to a patient having such breast cancer an effective amount of an ADC described herein. In one embodiment is a method of treating advanced hormone receptor positive (HR+) breast cancer or metastatic hormone receptor positive (HR+) breast cancer, also named advanced ER+ breast cancer or metastatic ER+ breast cancer by administering to a patient having such breast cancer an effective amount of an ADC described herein. In one embodiment, is a method of treating hormone receptor positive (HR+) breast cancer or estrogen receptor positive (ER+) breast cancer by administering to a patient having such breast cancer an effective amount of an ADC described herein.

In particular, ADCs described herein can be used either alone or in combination with standard of care treatment options for hormone receptor positive (HR+) breast cancer or estrogen receptor positive (ER+) breast cancer, which in general include surgery, systemic chemotherapy (either pre- or post-operatively) and/or radiation therapy. Depending on tumor and patient characteristics, systemic chemotherapy may be administered as adjuvant (post-operative) therapy or as neoadjuvant (pre-operative) therapy. In one embodiment, is a method of treating receptor positive (HR+) breast cancer or estrogen receptor positive (ER+) breast cancer by administering to a patient having such a breast cancer an effective amount of an ADC described herein and administering an effective amount of tamoxifen. In one embodiment is a method of treating receptor positive (HR+) breast cancer or estrogen receptor positive (ER+) breast cancer by administering to a patient having such a breast cancer an effective amount of an ADC described herein and administering an effective amount of an aromatase inhibitor, such as anastrozole, letrozole or exemestane. In one embodiment is a method of treating receptor positive (HR+) breast cancer or estrogen receptor positive (ER+) breast cancer by administering to a patient having such a breast cancer an effective amount of an ADC described herein and administering an effective amount of at least one additional therapeutic agent such as anastrozole, letrozole, exemestane and everolimus, palbociclib and letrozole, pablociclib and letrozole, fulvestrant, tamoxifen, toremifene, megestrol acetate, fluoxemesterone, and/or ethinyl estradiol.

In one embodiment is a method of treating a metastatic breast cancer in a patient having metastatic breast cancer by administering an effective amount of an ADC described herein and an effective amount of doxorubicin, pegylated liposomal doxorubicin, epirubicin, cyclophosphamide, carboplatin, cisplatin, docetaxel, paclitaxel, albumin-bound paclitaxel, capecitabine, gemcitabine, vinorelbine, eribulin, ixabepilone, methotrexate and 5-fluorouracil (5-FU). In one embodiment is a method of treating a metastatic breast cancer in a patient having metastatic breast cancer by administering an effective amount of an ADC described herein and an effective amount of docetaxel and capecitabine. In one embodiment is a method of treating a metastatic breast cancer in a patient having metastatic breast cancer by administering an effective amount of an ADC described herein and an effective amount of gemcitabine and paclitaxel.

In one embodiment is a combination therapy comprising an ADC described herein and doxorubicin, pegylated liposomal doxorubicin, epirubicin, cyclophosphamide, carboplatin, cisplatin, docetaxel, paclitaxel, albumin-bound paclitaxel, capecitabine, gemcitabine, vinorelbine, eribulin, ixabepilone, methotrexate and 5-fluorouracil (5-FU) for use in the treatment of metastatic breast cancer. In one embodiment is a combination therapy comprising an ADC described herein and docetaxel and capecitabine for use in the treatment of metastatic breast cancer. In one embodiment is a combination therapy comprising an ADC described herein and gemcitabine and paclitaxel for use in the treatment of metastatic breast cancer.

In another embodiment is a method of treating a breast cancer described herein by administering to such a patient an effective amount of ADC described herein and an effective amount of docetaxel, carboplatin and trastuzumab (TCH chemotherapy). In another embodiment is a method of treating a breast cancer described herein by administering to such a patient an effective amount of ADC described herein and an effective amount of docetaxel, carboplatin, trastuzumab and pertuzumab. In another embodiment is a method of treating a breast cancer described herein by administering to such a patient an effective amount of ADC described herein and an effective amount of 5-fluorouracil, epirubicin and cyclophosphamide (FEC chemotherapy and pertuzumab, trastuzumab and docetaxel or paclitaxel. In another embodiment is a method of treating a breast cancer described herein by administering to such a patient an effective amount of ADC described herein and an effective amount of paclitaxel and trastuzumab. In another embodiment is a method of treating a breast cancer described herein by administering to such a patient an effective amount of ADC described herein and an effective amount of pertuzumab and trastuzumab and paclitaxel or docetaxel.

In still another embodiment, the methods and combination therapy described herein comprise administering an effective amount of an ADC described herein and administering an effective amount of a taxane and a VEGF inhibitor (e.g., anti-VEGF antibody). For instance, in one embodiment, the methods and combination therapy described herein comprise administering an effective amount of an ADC described herein and administering an effective amount of paclitaxel and bevacizumab.

It is understood that the ADCs useful in the methods described herein comprise an antibody which can be selected from the therapeutic antibodies provided herein.

### EMBODIMENTS

It is understood that modifications that do not substantially affect the activity of the various embodiments described herein are also included. The following embodiments are intended to illustrate but not limit the present invention.

Embodiment 1. A BPA peptide composition comprising a peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11.

Embodiment 2. The BPA peptide composition of embodiment 1, wherein the BPA peptide is BPA7 (SEQ ID NO:8).

Embodiment 3. The BPA peptide composition of embodiment 1, wherein the BPA peptide is BPA10 (SEQ ID NO:11).

Embodiment 4. The BPA peptide composition of embodiment 1, wherein the BPA peptide is BPA 3 (SEQ ID NO:4) or BPA4 (SEQ ID NO:5)

Embodiment 5. A PhL peptide composition comprising a peptide comprising SEQ ID NO:12, SEQ ID NO:13, SEQ ID NO:14, SEQ ID NO:14, SEQ ID NO:15, SEQ ID NO:16, SEQ ID NO:17, SEQ ID NO:18, or SEQ ID NO:19, SEQ ID NO:20.

Embodiment 6. A Tdf peptide composition comprising a peptide comprising SEQ ID NO:21, SEQ ID NO:22, SEQ ID NO:23, SEQ ID NO:24, SEQ ID NO:25, SEQ ID NO:26, SEQ ID NO:27, SEQ ID NO:28, or SEQ ID NO:29.

Embodiment 7. An antibody-drug conjugate comprising
(i) an antibody; and
(ii) a BPA peptide of embodiment 1 covalently attached in the Fc portion of the antibody.

Embodiment 8. The antibody-drug conjugate composition of embodiment 3 having Formula (I):

Ab [symbol] B-E-L-D)ₚ (I)
wherein:
Ab is an antibody;
   B is a BPA peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11 covalently attached to the Fc region of the antibody and to L; E is an optional extension moiety as provided herein; L is a linker moiety;
   D is a drug moiety comprising a radiolabel, an antibody, or an anti-cancer agent such as a tubulin inhibitor, a topoisomerase II inhibitor, a DNA crosslinking cytoxic agent, an alkylating agent, a taxane, or an anthracycline agent; and
p is 1 or 2.

Embodiment 9. The antibody-drug conjugate composition of embodiment 7 comprising a homogenous mixture of antibody-drug conjugates wherein p is 2.

Embodiment 10. The antibody-drug conjugate composition of any one of embodiments 7-9, wherein the antibody is a monoclonal, IgG antibody.

Embodiment 11. The antibody-drug conjugate composition of any one of embodiments 7-10 wherein the antibody is a cysteine-engineered antibody.

Embodiment 12. The antibody-drug conjugate of any one of embodiments 7-10, wherein Ab is trastuzumab or trastuzumab emtansine.

Embodiment 13. The antibody-drug conjugate of any one of embodiments 7-12, wherein D is a maytansinoid, dolastatin, auristatin, calicheamicin, pyrrolobenzodiazepine dimer (PBD dimer), an anthracycline agent, duocarmycin, a synthetic duocarmycin analogue, a 1,2,9,9a-Tetrahydrocyclopropa[c]benzo[e]indol-4-one (CBI) dimer, a vinca alkaloid, a taxane (e.g. paclitaxel or docetaxel), trichothecene, camptothecin, silvestrol, or elinafide.

Embodiment 14. The antibody-drug conjugate of any one of embodiments 7-13, wherein D is a duocarmycin comprising mycarosylprotylonolide.

Embodiment 15. The antibody-drug conjugate of any one of embodiments 7-13, wherein D is a PBD dimer.

Embodiment 16. The antibody-drug conjugate of any one of embodiments 7-13, wherein D is a CBI dimer.

Embodiment 17. The antibody-drug conjugate of any one of embodiments 7-13, wherein D is an auristatin comprising MMAE or MMAF.

Embodiment 18. The antibody-drug conjugate of any one of embodiments 7-13, wherein D is an anthracycline agent comprising PNU-159682, doxorubicin, daunorubicin, epirubicin, idarubicin, mitoxantrone, or valrubicin.

Embodiment 19. The antibody-drug conjugate of any one of embodiments 7-13, wherein D is conjugated to a radiolabel.

Embodiment 20. The antibody-drug conjugate of any one of embodiments 7-12, wherein the radiolabel is ¹¹C, ¹³N, ¹⁵O, ¹⁸F, ³²P, ⁵¹Cr, ⁵⁷Co, ⁶⁴Cu, ⁶⁷Ga, ⁷⁵Se, ^{81m}Kr, ⁸²Rb, ^{99m}Tc, ¹²³I, ¹²⁵I, ¹³¹I, ¹¹¹In, or ²⁰¹Ti.

Embodiment 21. The antibody-drug conjugate of any one of embodiments 7-20, wherein L comprises formula (IV):

-Str-(Pep)ₘ-(Y)ₙ- (IV)
wherein,
Str is a stretcher unit or S covalently attached the BPA peptide;
Pep is an optional peptide unit of two to twelve amino acid residues;
Y is an optional spacer unit covalently attached to D; and
m and n are independently selected from 0 and 1.

Embodiment 22. The antibody conjugation of embodiment 21, wherein Str comprises a maleimidyl, bromacetamidyl or iodoacetamidyl moiety.

Embodiment 23. The antibody conjugation of embodiment 21 or 22, wherein Str has the formula (V):

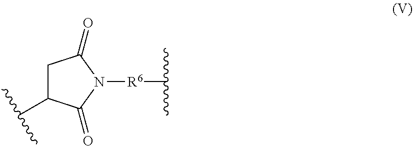

wherein,
R⁶ comprises C₁-C₁₂ alkylene, C₁-C₁₂ alkylene-C(═O), C₁-C₁₂ alkylene-NH, (CH₂CH₂O)ᵣ, (CH₂CH₂O)ᵣ—C(═O), (CH₂CH₂O)ᵣ—CH₂, or C₁-C₁₂ alkylene-NHC(═O)CH₂CH (thiophen-3-yl);
r is an integer ranging from 1 to 12; and
R⁶ is attached to Pep or Y.

Embodiment 24. The antibody-drug conjugate of any one of embodiments 21-23, wherein pep comprises a peptidomimetic moiety comprising:

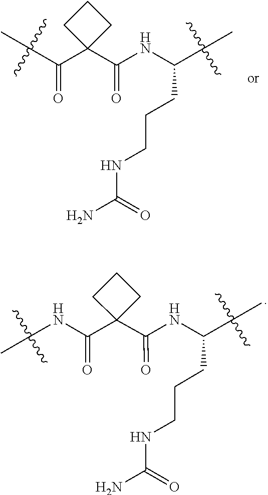 [SMILES: *C(=O)C1(C(=O)N[C@H](*)CCCNC(N)=O)CCC1 ; *NC(=O)C1(C(=O)N[C@H](*)CCCNC(N)=O)CCC1]

Embodiment 25. The antibody-drug conjugate of any one of embodiments 7-24, wherein, L comprises formula (IV) where R₆ is (CH₂)₅, Pep is val-cit, sq-cit, or nsq-cit, and Y is p-aminobenzyloxycarbonyl (PAB).

Embodiment 26. The antibody-drug conjugate of any one of embodiments 7-20, wherein L comprises the formula (VI):

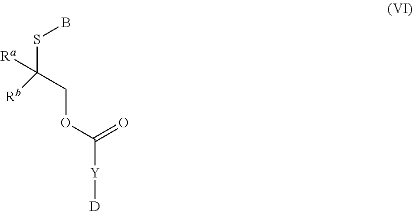

wherein,
B is a BPA peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11 covalently attached to the Fc region of the antibody and to L;
Y is para-aminobenzyl, p-aminobenzyloxycarbonyl (PAB), 2-aminoimidazol-5-methanol derivatives, ortho- or para-aminobenzylacetals, 4-aminobutyric acid amides, bicyclo[2.2.1] and bicyclo[2.2.2] ring systems, or 2-aminophenylpropionic acid amides; and
R^{a} and R^{b} are independently selected from H and C₁₋₃ alkyl, wherein only one of R^{a} and
R^{b} can be H, or R^{a} and R^{b} together with the carbon atom to which they are bound form a four- to six-membered ring optionally comprising an oxygen heteroatom.

Embodiment 27. The antibody-drug conjugate of embodiment 26, wherein Y is para-aminobenzyl or p-aminobenzyloxycarbonyl.

Embodiment 28. The antibody-drug conjugate of any one of embodiments 7-20, wherein,

B is BPA7 (SEQ ID NO:8);

Ab is Trastuzumab;

D is MMAE or MMAF; and

L comprises a compound of formula (IV):

-Str-(Pep)ₘ-(Y)ₙ- (IV)
wherein Str is a compound of formula (V):

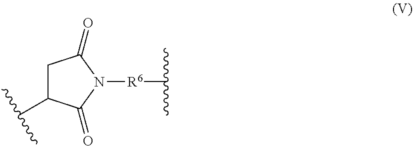

wherein, R6 is (CH2)₅,
Pep is val-cit, sq-cit, or nsq-cit; and
Y is p-aminobenzyloxycarbonyl (PAB).

Embodiment 29. The antibody-drug conjugate of any one of embodiments 7-28, wherein the antibody binds to a tumor-associated antigen or cell-surface receptor.

Embodiment 30. The antibody-drug conjugate of embodiment 29, wherein the tumor-associated antigen or cell-surface receptor is selected from the group consisting of (1)-(53):
(1) BMPR1B (bone morphogenetic protein receptor-type IB);
(2) E16 (LAT1, SLC7A5);
(3) STEAP1 (six transmembrane epithelial antigen of prostate);
(4) MUC16 (0772P, CA125);
(5) MPF (MPF, MSLN, SMR, megakaryocyte potentiating factor, mesothelin);
(6) Napi2b (NAPI-3B, NPTIIb, SLC34A2, solute carrier family 34 (sodium phosphate), member 2, type II sodium-dependent phosphate transporter 3b);
(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B);
(8) PSCA hlg (2700050C12Rik, C530008O16Rik, RIKEN cDNA 2700050C12, RIKEN cDNA 2700050C12 gene);
(9) ETBR (Endothelin type B receptor);
(10) MSG783 (RNF124, hypothetical protein FLJ20315);
(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein);
(12) TrpM4 (BR22450, FLJ20041, TRPM4, TRPM4B, transient receptor potential cation channel, subfamily M, member 4);
(13) CRIPTO (CR, CR1, CRGF, CRIPTO, TDGF1, teratocarcinoma-derived growth factor);
(14) CD21 (CR2 (Complement receptor 2) or C3DR (C3d/Epstein Barr virus receptor) or Hs 73792);
(15) CD79b (CD79B, CD79β, IGb (immunoglobulin-associated beta), B29);
(16) FcRH2 (IFGP4, IRTA4, SPAP1A (SH2 domain containing phosphatase anchor protein 1a), SPAP1B, SPAP1C);
(17) HER2;
(18) NCA;
(19) MDP;
(20) IL20Rα;
(21) Brevican;
(22) EphB2R;
(23) ASLG659;
(24) PSCA;
(25) GEDA;
(26) BAFF-R (B cell-activating factor receptor, BLyS receptor 3, BR3);
(27) CD22 (B-cell receptor CD22-B isoform);
(28) CD79a (CD79A, CD79α, immunoglobulin-associated alpha);
(29) CXCR5 (Burkitt's lymphoma receptor 1);
(30) HLA-DOB (Beta subunit of MHC class II molecule (Ia antigen));
(31) P2X5 (Purinergic receptor P2X ligand-gated ion channel 5);
(32) CD72 (B-cell differentiation antigen CD72, Lyb-2);
(33) LY64 (Lymphocyte antigen 64 (RP105), type I membrane protein of the leucine rich repeat (LRR) family);
(34) FcRH1 (Fc receptor-like protein 1);
(35) FcRH5 (IRTA2, Immunoglobulin superfamily receptor translocation associated 2);
(36) TENB2 (putative transmembrane proteoglycan);
(37) PMEL17 (silver homolog; SILV; D12S53E; PMEL17; SI; SIL);
(38) TMEFF1 (transmembrane protein with EGF-like and two follistatin-like domains 1; Tomoregulin-1);
(39) GDNF-Ra1 (GDNF family receptor alpha 1; GFRA1; GDNFR; GDNFRA; RETL1; TRNR1; RET1L; GDNFR-alpha1; GFR-ALPHA-1);
(40) Ly6E (lymphocyte antigen 6 complex, locus E; Ly67, RIG-E, SCA-2,TSA-1);
(41) TMEM46 (shisa homolog 2 (Xenopus laevis); SHISA2);
(42) Ly6G6D (lymphocyte antigen 6 complex, locus G6D; Ly6-D, MEGT1);
(43) LGR5 (leucine-rich repeat-containing G protein-coupled receptor 5; GPR49, GPR67);
(44) RET (ret proto-oncogene; MEN2A; HSCR1; MEN2B; MTC1; PTC; CDHF12; Hs.168114; RET51; RET-ELE1);
(45) LY6K (lymphocyte antigen 6 complex, locus K; LY6K; HSJ001348; FLJ35226);
(46) GPR19 (G protein-coupled receptor 19; Mm.4787);
(47) GPR54 (KISS1 receptor; KISS1R; GPR54; HOT7T175; AXOR12);
(48) ASPHD1 (aspartate beta-hydroxylase domain containing 1; LOC253982);
(49) Tyrosinase (TYR; OCAIA; OCA1A; tyrosinase; SHEP3);
(50) TMEM118 (ring finger protein, transmembrane 2; RNFT2; FLJ14627);
(51) GPR172A (G protein-coupled receptor 172A; GPCR41; FLJ11856; D15Ertd747e);
(52) CD33; and
(53) CLL-1.

Embodiment 31. A pharmaceutical composition comprising the antibody-drug conjugate composition according to any one of embodiments 7-30 and a pharmaceutically acceptable excipient.

Embodiment 32. A method of treating lung cancer, bladder cancer, renal cell cancer (RCC), melanoma, or breast cancer, the method comprising administering to said patient an effective amount of an antibody-drug conjugate of any one of embodiments 7-30.

Embodiment 33. A method of treating breast cancer, the method comprising administering to a patient having said breast cancer an effective amount of an antibody-drug conjugate of any one of embodiments 7-30.

Embodiment 34. A method of treating lung cancer, the method comprising administering to a patient having said lung cancer an effective amount of an antibody-drug conjugate of any one of embodiments 7-30.

Embodiment 35. The method of embodiment 34, wherein the lung cancer is non-small cell lung cancer.

Embodiment 36. A method of treating bladder cancer, the method comprising administering to a patient having said bladder cancer an effective amount of an antibody-drug conjugate of any one of embodiments 7-30.

Embodiment 37. A method of treating kidney cancer, the method comprising administering to a patient having said kidney cancer an effective amount of an antibody-drug conjugate of any one of embodiments 7-30.

Embodiment 38. The method of any one of embodiments 32-38, wherein the antibody-drug conjugate is co-administered with another anticancer agent.

Embodiment 39. The method of embodiment 38, wherein the anticancer agent comprises one or more therapeutic antibodies.

Embodiment 40. The method of embodiment 38, wherein the anticancer agent is radiation therapy or chemotherapy.

Embodiment 41. A method of imaging a patient for a tumor, the method comprising administering to the patient a composition comprising an ADC of any one of embodiments 7-30 and detecting the quantity and location of the label.

Embodiment 42. The method of embodiment 41, wherein the label comprises ¹¹C, ¹³N, ¹⁵O, ¹⁸F, ³²P, ⁵¹Cr, ⁵⁷Co, ⁶⁴Cu, ⁶⁷Ga, ⁷⁵Se, ^{81m}Kr, ⁸²Rb, ^{99m}Tc, ¹²³I, ¹²⁵I, ¹³¹I, ¹¹¹In, or ²⁰¹Ti.

Embodiment 43. A method to prepare an antibody-drug conjugate composition of any one of any one of embodiments 7-30, the method comprising:

(i) reacting an antibody under photo-crosslinking conditions with a BPA peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11 thereby forming an antibody conjugate;

(ii) optionally removing a protecting group on the terminal end of the BPA peptide;

(iii) reacting the antibody conjugate with a drug (D) further comprising a linker to form the antibody-drug conjugate composition having Formula I, wherein the linker comprises formula (IV):

-Str-(Pep)ₘ-(Y)ₙ- (IV)
wherein,
Str is a stretcher unit or S covalently attached the BPA peptide;
Pep is an optional peptide unit of two to twelve amino acid residues;
Y is an optional spacer unit covalently attached to D; and
m and n are independently selected from 0 and 1.

Embodiment 44. The method of embodiment 43, wherein the antibody is a monoclonal, IgG antibody.

Embodiment 45. The method of embodiment 43 or 44, wherein the antibody is a cysteine-engineered antibody.

Embodiment 46. The method of any one of embodiments 43-45, wherein the antibody binds to a tumor-associated antigen or cell-surface receptor.

Embodiment 47. The method of any one of embodiments 43-46, wherein the BPA peptide is BPA7 (SEQ ID NO:8).

Embodiment 48. The method of any one of embodiments 43-47, wherein the BPA peptide further comprises an extension moiety comprising PEG.

Embodiment 49. The method of embodiment 48, wherein the extension moiety is PEG₁₂-SATA or SATA.

Embodiment 50. The method of any one of embodiments 43-49, wherein photo-crosslinking conditions comprise irradiating under ultraviolet (UV) light.

Embodiment 51. The method of any one of embodiments 43-50, wherein the antibody and the BPA peptide are irradiated with 365 nm UV light.

Embodiment 52. The method of any one of embodiments 43-51, wherein the photo-crosslinking conditions comprise irradiating the antibody and the BPA peptide in a multi-well plate.

Embodiment 53. The method of any one of embodiments 43-52, wherein photo-crosslinking conditions further comprise an antioxidant.

Embodiment 54. The method of embodiment 53, wherein the antioxidant is selected from the group consisting of 5-hydroxyindole (5-HI), methionine, sodium thiosulfate, catalase, platinum, tryptophan, 5-methoxy-tryptophan, 5-amino-tryptophan, 5-fluoro-tryptophan, N-acetyl tryptophan, tryptamine, tryptophanamide, serotonin, melatonin, kynurenine, indolyl derivatives, salicylic acid, 5-hydroxy salicylic acid, anthranilic acid, and 5-hydroxy anthranilic acid.

Embodiment 55. A method to prepare an antibody-drug conjugate composition of any one of any one of embodiments 7-30, the method comprising reacting an antibody under photo-crosslinking conditions with a BPA peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11, wherein the BPA peptide is covalently attached to a drug moiety (D) through a linker comprising formula (IV):

-Str-(Pep)ₘ-(Y)ₙ- (IV)
wherein,
Str is a stretcher unit or S covalently attached the BPA peptide;
Pep is an optional peptide unit of two to twelve amino acid residues;
Y is an optional spacer unit covalently attached to D; and
m and n are independently selected from 0 and 1,
   thereby forming an antibody conjugate.

Embodiment 56. The method of embodiment 55, wherein the antibody is a monoclonal, IgG antibody.

Embodiment 57. The method of embodiment 55 or 56, wherein the antibody is a cysteine-engineered antibody.

Embodiment 58. The method of any one of embodiments 55-57, wherein the antibody binds to a tumor-associated antigen or cell-surface receptor.

Embodiment 59. The method of any one of embodiments 55-58, wherein the BPA peptide is BPA7 (SEQ ID NO:8).

Embodiment 60. The method of any one of embodiments 55-59, wherein the BPA peptide further comprises an extension moiety comprising PEG.

Embodiment 61. The method of embodiment 60, wherein the extension moiety is PEG₁₂-SATA or SATA.

Embodiment 62. The method of any one of embodiments 55-61, wherein photo-crosslinking conditions comprise irradiating under ultraviolet (UV) light.

Embodiment 63. The method of any one of embodiments 55-62, wherein the antibody and the BPA peptide are irradiated with 365 nm UV light.

Embodiment 64. The method of any one of embodiments 55-63, wherein the photo-crosslinking conditions comprise irradiating the antibody and the BPA peptide in a multi-well plate.

Embodiment 65. The method of any one of embodiments 55-64, wherein photo-crosslinking conditions further comprise an antioxidant.

Embodiment 66. The method of embodiment 65, wherein the antioxidant is selected from the group consisting of 5-hydroxyindole (5-HI), methionine, sodium thiosulfate, catalase, platinum, tryptophan, 5-methoxy-tryptophan, 5-amino-tryptophan, 5-fluoro-tryptophan, N-acetyl tryptophan, tryptamine, tryptophanamide, serotonin, melatonin, kynurenine, indolyl derivatives, salicylic acid, 5-hydroxy salicylic acid, anthranilic acid, and 5-hydroxy anthranilic acid.

The following Examples are presented by way of illustration, not limitation. Compounds described herein were synthesized using the schemes and procedures provided herein. Chemicals and reagents were of high grade unless otherwise noted.

### EXAMPLES

Example 1: Peptide synthesis. Peptides were synthesized via standard Fmoc solid-phase peptide synthesis methods, purified to >90% by reverse-phase HPLC and lyophilized prior to use in conjugation reactions (Elim Biopharmaceuticals).

For synthesis of SATA-BPA7, approximately 10 mg of des-acetyl BPA7 (600 μL; 10 mM in DMSO) was reacted with N-succinimidyl S-acetylthioacetate (SATA, ThermoFisher) (600 μL; 10 mM in DMSO) and N,N-diisopropylethylamine (DIEA) (300 μL; 20 mM in DMSO) at room temperature for 2 hours. The resulting SATA-BPA7 peptide was purified by preparative reverse-phase HPLC using a C18 column with a gradient of buffer B (0.1% TFA in acetonitrile) in buffer A (0.1% TFA in water). Fractions were pooled and assessed for presence of the product and purity by LC-MS. Pooled fractions were lyophilized to obtain ˜1.8 mg of the final product. Preparation of SATA-PEG-BPA7 from 10 mg of des-acetyl BPA7 and S-acetyl-dPEG₁₂-NHS ester (Quanta Biodesign) proceeded in a similar fashion (FIG. 11).

Example 2: Antibody conjugation. Conjugation reactions for photocrosslinking peptides were performed open and uncovered in V-bottom, clear, polystyrene 96-well plates (ThermoFisher, product #2605) with a final reaction volume of 50 μL. Unused wells were filled with 150 μL of deionized water. Optimized reactions were performed in 20 mM histidine acetate buffer, pH=5.5, with a final concentration of 48 μM antibody, 480 μM photo-crosslinking peptide, 480 μM 5-hydroxyindole (5-HT, Sigma-Aldrich), with 11% (v/v) DMSO. Photocrosslinking was initiated upon UV irradiation at 365 nm in a UVP-crosslinker chamber (AnalytikJena, CL-1000L) for 4 hours with plates on a gel ice pack refrigerated at 4 C. The DAR was assessed by LC-MS analysis of Fc/2 or heavy chain fragments generated by IdeS or DTT treatments of Trastuzumab, respectively.

To prepare MMAE-linked ADCs, Trastuzumab was conjugated to SATA-BPA7 and SATA-PEG-BPA7 using the optimized photocrosslinking reaction conditions described above. The resulting conjugates were treated with 50 mM hydroxylamine for 30 min at room temperature to effect removal of the acetyl groups and liberation of the free thiols on the conjugated peptides, as indicated by LC-MS. The deprotected Trastuzumab/SATA-BPA7 or Trastuzumab/SATA-PEG-BPA7 conjugates were purified with strong cation exchange spin columns (Pierce). Cation exchange columns were equilibrated with 20 mM histidine acetate, pH 5.5. The conjugated antibody samples, diluted first into equilibration buffer (histidine-acetate, pH 5.5), were bound to the column, washed with equilibration buffer and eluted with 20 mM histidine acetate, pH 5.5, 300 mM NaCl.

Conjugation of mc-vc-PAB-MMAE (malemide-val-cit-PAB-MMAE) to thiol-deprotected Trastuzumab/SATA-PEG-BPA7 was carried out with 4 molar equivalents (relative to antibody) of mc-vc-PAB-MMAE in 50 mM Tris, pH 7.5 buffer with 10% (v/v) DMF overnight at room temperature. The resulting MMAE conjugates were purified by S maxi cation exchange columns and were characterized by LC-MS and SEC using TSKgel G3000SWxl column (TOSOH) to determine DAR, aggregation and final ADC concentration.

Example 2: SPR binding experiments. Kinetics of peptide binding to Trastuzumab were measured by surface plasmon resonance (SPR) on a Biacore 3000 instrument (GE Healthcare) using a previously established method. (Gong, Y.; et al., Development of the Double Cyclic Peptide Ligand for Antibody Purification and Protein Detection. Bioconjugate chemistry 2016). An amine coupling kit (GE Healthcare) was used to immobilize Trastuzumab to the surface of a CM5 Sensor Chip (GE Healthcare). All injections were double-referenced with real-time reference channel subtraction and buffer blank injections. Data were analyzed using the BiaEvaluation software (version 4.1, GE Healthcare).

To determine the inhibition of FcRn binding to human IgG1 by Fc-III peptide, surface plasmon resonance (SPR) measurement with a BIAcore™ 8K instrument was used. Briefly, purified recombinant human IgG1 was captured on a series S protein A sensor chip. Serial dilutions of Fc-III peptide with 1 μM FcRn in assay buffer (10 mM MES pH 6.0, 150 mM NaCl, 0.05% Tween-20) were injected on the sensor chip at a flow rate of 30 μL/minute for 6 minutes, which allowed the system to be at steady-state for all concentrations. The SPR response was then measured, plotted against the concentration of peptide and an IC₅₀ nonlinear fit was performed, restraining the top of the curve to the response of FcRn alone, using GraphPad Prism version 7.0c for Mac OS X (GraphPad Software, La Jolla Calif. USA, www.graphpad.com).

Example 3: X-ray crystallography. Human Fc for crystallization studies was prepared from limited digestion with lysine C (Wako) of Trastuzumab into Fab and Fc domains, the latter of which was purified by cation exchange chromatography on an Akta purification system (GE Healthcare). The purified Fc domain was concentrated to 20 mg/mL using a 10 k Amicon centrifugal concentrator (EMD Millipore). Conjugation of the IgG1-Fc sample to BPA7 was performed using standard reaction conditions (see above) and the conjugate was purified by size-exclusion chromatography (SEC). Monomeric BPA7/Fc conjugate was pooled and concentrated to a final concentration of 6 mg/mL using a 10 k Amicon centrifugal concentrator. The quality of the final conjugate was assessed by SDS-PAGE, SEC and LC-MS to ensure high purity and DAR (DAR=1.9, 96.6% monomeric).

Crystals of the photoconjugate were grown at 18° C. by mixing 2 μL of 100 mM sodium acetate pH=5.6, 12% (w/v) PEG 1000 with 1 μL of 6 mg/mL BPA7/Fc conjugate by hanging-drop vapor diffusion with 1 mL reservoirs. Crystals grew as thin plates after 1 week and were cryo-stabilized in 30% (v/v) ethylene glycol and flash frozen in liquid nitrogen. Data were collected to Bragg diffraction limit of 2.3 Å at the ALS 5.0.2 and processed with XDS in space group P2₁ and unit cell of a=66.11 b=60.85 c=68.17 90.00, 103.13, 90.00. (absch, W., Integration, scaling, space-group assignment and post-refinement. Ada crystallographica. Section D, Biological crystallography 2010, 66 (Pt 2), 133-144). Molecular replacement was performed with a previous structure of Fc-III bound to the human Fc domain (PDB code: 1DN2) as the search model and using Phaser from the CCP4 suite. (McCoy, A. J.; Grosse-Kunstleve, R. W.; Adams, P. D.; Winn, M. D.; Storoni, L. C.; Read, R. J., Phaser crystallographic software. Journal of applied crystallography 2007, 40 (Pt 4), 658-674). Refinement was performed using Phenix with rounds of manual fitting using Coot. (dams, P. D.; Afonine, P. V.; Bunkóczi, G.; Chen, V. B.; Davis, I. W.; Echols, N.; Headd, J. J.; Hung, L.-W.; Kapral, G. J.; Grosse-Kunstleve, R. W.; McCoy, A. J.; Moriarty, N. W.; Oeffner, R.; Read, R. J.; Richardson, D. C.; Richardson, J. S.; Terwilliger, T. C.; Zwart, P. H., PHENIX: a comprehensive Python-based system for macromolecular structure solution. Acta crystallographica. Section D, Biological crystallography 2010, 66 (Pt 2), 213-221: sley, P.; Lohkamp, B.; Scott, W. G.; Cowtan, K., Features and development of Coot. Acta crystallographica. Section D, Biological crystallography 2010, 66 (Pt 4), 486-501). The resolution of the final refined model was 2.58 Å with a Rcryst and Rfree of 0.226 and 0.261, respectively (Table 9).

TABLE 9
Diffraction data and structure refinement statistics

| PDB code | 6N9T | |
|---|---|---|
| Space group | P2₁ | |
| Unit cell | a = 66.1 Å, b = 60.9 Å, c = 68.2 Å, | |
| | α = 90°, β = 103°, γ = 90° | |
| Resolution | 2.58 | Å |
| Total measured reflections | 16883 | (173) ¹ |
| Completeness (%) | 100 | (99.4) |
| Redundancy | 3.4 | (3.4) |
| I/σ | 8.6 | (2.1) |
| Rsym² | 0.117 | (0.281) |
| Resolution range | 50-2.58 | Å |
| Rcryst³/Rfree⁴ | 0.226/0.261 | |
| Non-hydrogen atoms | 3911 | |
| Water molecules | 186 | |
| Average B | 43.7 | Å² |
| r.m.s.d. bond lengths | 0.003 | Å |
| r.m.s.d. angles | 0.687° | |

| | |
|---|---|
| Ramachandran | 0.916/0.084/0/0 |

Example 4: Measuring oxidation of Met-252 and impacts on photoconjugation. Trastuzumab in storage buffer (5 mM L-histidine, 60 mM trehalose, 0.01% polysorbate, pH=6) was treated with 5% (w/v) of 2,2′-azobis(2-methylpropionamidine) (AAPH, Sigma-Aldrich) at 37° C. in a covered reaction vessel. (olzer, E.; Diepold, K.; Bomans, K.; Finkler, C.; Schmidt, R.; Bulau, P.; Huwyler, J.; Mahler, H. C.; Koulov, A. V., Selective Oxidation of Methionine and Tryptophan Residues in a Therapeutic IgG1 Molecule. J Pharm Sci 2015, 104 (9), 2824-31). Addition of AAPH increased the pH of the solution, so 1 M sodium acetate, pH=5, was added to a final concentration of 100 mM to bring the pH of both the AAPH oxidized and unoxidized control samples to ˜5. Aliquots were extracted for each time point (0, 1, 4.5, 24, 123 hours) and buffer exchanged to remove excess AAPH using S maxi cation exchange columns (Thermofisher), eluting with phosphate buffered saline (PBS). Samples were then subjected to photocrosslinking with peptide BPA7 using standard reaction conditions.

Methionine oxidation on AAPH from 0 to 24 hours was determined by LC-MS/MS on the digested protein. 20 ug of the 20 mg/mL Trastuzumab AAPH timepoint samples were diluted with 50 mM ammonium bicarbonate pH 8 (Burdick and Jackson, Muskegon, Mich.) then digested with modified trypsin (Promega, Madison, Wis.) at a 1:50 enzyme:substrate ratio for 3 hours at 37° C. Digestions were quenched with 4 ul of 2% trifluoroacetic acid and then subjected to c18 stage-tip clean up. Samples were injected via an auto-sampler onto a 75 μm×100 mm column (BEH, 1.7 micron, Waters Corp) at a flow rate of 1 μL/min using a NanoAcquity UPLC (Waters Corp). A gradient from 98% solvent A (water +0.1% formic acid) to 80% solvent B (acetonitrile+0.08% formic acid) was applied over 40 min. Samples were analyzed on-line via nanospray ionization into Q-Exactive HF Orbitrap mass spectrometer (Thermo Fisher Scientific, San Jose, Calif.). Data was collected in data dependent mode with the top 15 most abundant ions selected for fragmentation to generate HCD spectra. Tandem mass spectrometric data were analyzed using Byonic™ (Protein Metrics Inc, San Carlos, Calif.) software and interrogated with Byologic™ (Protein Metrics Inc., San Carlos, Calif.). The percentage of oxidized methionine at position 252 was determined by comparing the area under the curve (AUC) for the oxidized and unoxidized tryptic peptide, DTLMISR.

Example 5: Plasma Stability Analysis. To evaluate stability, photoconjugates were spiked into plasma or buffer (1×PBS [pH7.4], 0.5% BSA, 15 PPM Proclin) to a final concentration of 100 ug/mL. After mixing, 100 μL aliquots were incubated for different time points (0, 48 and 96 hour) at 37° C. in an incubator with shaking (˜700 rpm). After 48 and 96 hrs, samples were stored in a −80° C. freezer until AC LC-MS was performed as described previously. (Xu, K.; Liu, L.; Saad, O. M.; Baudys, J.; Williams, L.; Leipold, D.; Shen, B.; Raab, H.; Junutula, J. R.; Kim, A.; Kaur, S., Characterization of intact antibody-drug conjugates from plasma/serum in vivo by affinity capture capillary liquid chromatography-mass spectrometry. Analytical biochemistry 2011, 412 (1), 56-66). Briefly, washed streptavidin-coated (SA) magnetic beads (Thermo Fisher Scientific, Waltham, Mass.) were mixed with either biotinylated extracellular domain of target (e.g. human erb2) or anti-idiotypic antibody for specific capture using a KingFisher Flex (Thermo Fisher Scientific, Waltham, Mass.) and incubated for 2 hrs at room temperature with gentle agitation. After washing twice with HBS-EP buffer (GE Healthcare, Sunnyvale, Calif.), beads were added to stability samples diluted 16-fold and incubated for 2 hrs at room temperature with gentle agitation. After ADC affinity capture, beads were washed twice with HBS-EP buffer and deglycosylated overnight with PNGase F (New England BioLabs, Ipswich, Mass.). Following two more washes with HBS-EP buffer, two washes with water and a final wash with 10% acetonitrile, the ADCs were eluted from the beads with 30% acetonitrile/0.1% formic acid for 30 mins at room temperature with gentle agitation. The eluted samples were then analyzed by LC-MS (Synapt-G2S, Waters, Milford, Mass.) using a PepSwift reversed phase monolithic column (500 μm×5 cm) (Thermo Fisher Scientific, Waltham, Mass.) maintained at 65° C. using a Waters Acquity UPLC system at a flow rate of 20 μL/min with the following gradient: 20% B (95100% acetonitrile+0.1% formic acid) at 0-2 min; 35% B at 2.5 min; 65% B at 5 min; 95% B at 5.5 min; 5% B at 6 min. The column was directly coupled for online detection with Waters Synapt G2-S Q-ToF mass spectrometry operated in positive ESI mode with an acquisition range from m/z 500 to 5000. Stability analysis was performed using Waters BiopharmaLynx 1.3.3 software and a custom Vortex script (Dotmatics, Bishops Stortford, United Kingdom). The relative ratios of ADC with different DARs were calculated by dividing the intensity of the specific ADC species with the intensity from the total ADC species and % DAR calculated as previously described. (Xu, K.; Liu, L.; Saad, O. M.; Baudys, J.; Williams, L.; Leipold, D.; Shen, B.; Raab, H.; Junutula, J. R.; Kim, A.; Kaur, S., Characterization of intact antibody-drug conjugates from plasma/serum in vivo by affinity capture capillary liquid chromatography-mass spectrometry. Analytical biochemistry 2011, 412 (1), 56-66).

Example 6: Development of photoconjugation method. Mutants of the 13-residue cyclic peptide, Fc-III, discovered previously by phage display to bind to the human Fc domain with nanomolar affinity (FIG. 1) were prepared having a single amino acid mutation with BPA (See Table 1). (DeLano, W. L.; Ultsch, M. H.; Wells, J. A., Convergent solutions to binding at a protein-protein interface. Science 2000). Without being bound by any particular theory, positioning a Bpa residue in Fc-III that, upon complexation, would be nearby a suitably reactive residue on the Fc domain would enable efficient and site-specific peptide/antibody conjugation upon UV irradiation. The reactive radius of benzophenone has been estimated to be >10 angstroms. (Wittelsberger, A.; Mierke, D. F.; Rosenblatt, M., Mapping ligand-receptor interfaces: approaching the resolution limit of benzophenone-based photoaffinity scanning. Chemical biology &amp; drug design 2008, 71 (4), 380-383). Residue in WT Fc-III sequence, except for Trp-4 and Gly-7, were mutated to Bpa. These residues projected away from the Further comprising. The two cysteines, which form an intramolecular disulfide bridge shown to be critical for tight binding to the Further comprising, were also not mutated. (Kang, H. J.; Choe, W.; Min, J.-K.; Lee, Y.-m.; Kim, B. M.; Chung, S. J., Cyclic peptide ligand with high binding capacity for affinity purification of immunoglobulin G. Journal of chromatography. A 2016, 1466, 105-112). All peptides were synthesized by standard solid-phase peptide synthesis as described herein and purified by reverse-phase HPLC prior to conjugation evaluation.

Conjugation was initially performed by reacting the panel of Fc-III peptides BPA1-BPA9 with the human monoclonal antibody Trastuzumab (TMab) in PBS in micro-centrifuge tubes on ice under a hand-held 365 nm lamp for one hour. Upon monitoring by LCMS, a peak corresponding to the desired product was observed in the reaction with peptide BPA7 giving a drug-to-antibody ratio (DAR) of ˜0.04 (data not shown).

BPA peptides in a 96-well plate were reacted directly with TMab under a 365 nm lamp with minimal space between plate and lamp at room temperature. The new photocrosslinking conditions resulted in a DAR of ˜1.7 for peptide BPA7 after 4.5 hours of radiation. Conjugation was also observed for peptides BPA3 and BPA4 (DAR=0.2 and 1.2, respectively). These results suggested that duration and/or extent of exposure to the UV source has a strong impact on conjugation efficiency. Subsequent experiments were conducted in 96-well plates within a specialized UV photoreaction chamber to ensure even exposure of conjugation reaction mixtures to light.

Using the irradiation chamber, photoconjugation of BPA7 to the Fc domain of TMab resulted (DAR=1.8; FIG. 2A, Row A). The peak for the Fab′2 region of the antibody broadened by ˜5.4-fold relative to that of unreacted antibody (FIG. 2B, Row A). Irradiation of antibodies with UV light is known to cause radical-mediated oxidation of tryptophan and methionine residues. (Sreedhara, A.; Yin, J.; Joyce, M.; Lau, K.; Wecksler, A. T.; Deperalta, G.; Yi, L.; Wang, Y. J.; Kabakoff, B.; Kishore, R. S. K., Effect of ambient light on IgG1 monoclonal antibodies during drug product processing and development. European Journal of Pharmaceutics and Biopharmaceutics 2016, 100 (C), 38-46). Such effects can result in product heterogeneity and lead to reduction in performance in vitro or in vivo (e.g., due to reduced binding to antigen).

Effects of photoconjugation of BPA7 were minimized by further optimizing various parameters of the reaction. For example, cooling the 96-well reaction plate to ˜4° C. during irradiation reduced the relative Fab′2 peak width to 1.4, while maintaining DAR of 1.5 (FIG. 2B, Row B). Switching buffer from PBS at pH 7.4 to histidine-acetate acetate at pH 5.5 further reduced Fab′2 peak width ratio to 1.2 while increasing DAR to 1.8 (FIG. 2B, Row C). Including in the reaction mixture 5-hydroxyindole, an agent known to protect antibodies from UV-induced damage, reduced Fab′2 heterogeneity to near completion with a DAR=1.4. (FIG. 2B, Row D). (Grewal, P.; Mallaney, M.; Lau, K.; Sreedhara, A., Screening Methods to Identify Indole Derivatives That Protect against Reactive Oxygen Species Induced Tryptophan Oxidation in Proteins. Molecular pharmaceutics 2014, 11 (4), 1259-1272). DAR was increased under such conditions raising the concentration of peptide BPA7 in the reaction and extending the reaction time. Concentrations of peptide 10-fold higher than antibody and UV irradiation for 6 hours were sufficient to achieve DAR of 1.9 with minimal Fab modification (peak width ratio=1.1 FIG. 2B, Row E; FIG. 12).

Fc-III peptides incorporating residues with a diazirine photocrosslinking group instead of BPA were examined. Like benzophenone-based photoaffinity ligands, diazirine-bearing ligands can react with amino acid side chains on bound receptors upon UV irradiation. However, diazirines form carbenes instead of diradicals and have shown different reactivity trends across amino acid side chains relative to benzophenone photocrosslinkers. (Sigrist, H.; Mühlemann, M.; Dolder, M., Philicity of amino acid side-chains for photogenerated carbenes. Journal of Photochemistry and . . . 1990: Das, J., Aliphatic Diazirines as Photoaffinity Probes for Proteins: Recent Developments. Chemical reviews 2011, 111 (8), 4405-4417). Synthesis of PhL peptides PhL1-PhL9 and Tdf peptides Tdf1-Tdf9 was completed where either photo-Leu or Tdf, respectively, was placed at various positions (Table 1).

Diazirine peptides demonstrated detectable conjugation. The reaction was not as complete as reactions performed with BPA 7. (FIG. 10). Peptides incorporating photo-Leu were more efficiently conjugated to TMab than those with Tdf although. DAR above 0.4 was not obtained for either series.

Example 7: Biophysical and structural characterization of Bpa peptide binding and conjugation. The affinity of the BPA peptides BPA1-BPA9 and the parent peptide Fc-III for TMab was measured by surface plasmon resonance (SPR) (FIG. 3). The Fc-III peptide had a tested dissociation constant (K_{d}) of 17±0.2 nM (FIG. 3A), consistent with values reported previously for this peptide. (DeLano, W. L.; Ultsch, M. H.; Wells, J. A., Convergent solutions to binding at a protein-protein interface. Science 2000: Kang, H. J.; Choe, W.; Min, J.-K.; Lee, Y.-m.; Kim, B. M.; Chung, S. J., Cyclic peptide ligand with high binding capacity for affinity purification of immunoglobulin G. Journal of chromatography. A 2016, 1466, 105-112). In all cases, substitution of amino acids in Fc-III for the bulkier BPA residue to generate peptides BPA1-BPA9 resulted in reduced binding affinity from ˜27- to >4000-fold (FIG. 3B and FIG. 3C). The solvent accessible surface area of the substituted amino acid in the Fc-III peptide, measured from the published structure, was a reasonably strong predictor of loss in binding affinity for the associated BPA mutant (FIG. 13A). (DeLano, W. L.; Ultsch, M. H.; Wells, J. A., Convergent solutions to binding at a protein-protein interface. Science 2000).

There appeared to be no correlation between noncovalent binding affinity of peptides BPA1-BPA9 and conjugation efficiency (FIG. 13B). For example, BPA7 bound to TMab ˜150-fold less tightly than did BPA9 (K_{d}=70 uM versus 0.47 uM, respectively), yet BPA7 photoconjugated efficiently to the antibody (DAR=1.9) whereas peptide BPA9 did not (DAR=0.0; FIG. 3C).

A peptide variant of Fc-III containing an extra disulfide bridge was previously reported to have a significantly improved binding affinity to human IgG (K_{d}=2.5 nM for the analog versus 70 nM for Fc-III itself in the same publication). (Gong, Y.; Zhang, L.; Li, J.; Feng, S.; Deng, H., Development of the Double Cyclic Peptide Ligand for Antibody Purification and Protein Detection. Bioconjugate chemistry 2016). The analogous doubly-cyclized version of BPA7 (BPA10, Table 1) was synthesized and its affinity measured. BPA10 was further evaluated for conjugation to TMab. BPA10 displayed improved binding affinity versus peptide BPA7 (K_{d}=11.4 versus 70 μM) as expected, but photoconjugation efficiency was decreased relative to BPA7 (DAR=1.2 versus 1.9; FIG. 3C). These results suggested that the photoconjugation reaction between Fc-III BPA variants and TMab is not driven by the noncovalent affinity of the peptide/antibody complex per se, but rather by the precise positioning of the BPA moiety, suggesting a highly specific reaction with a residue in the antibody.

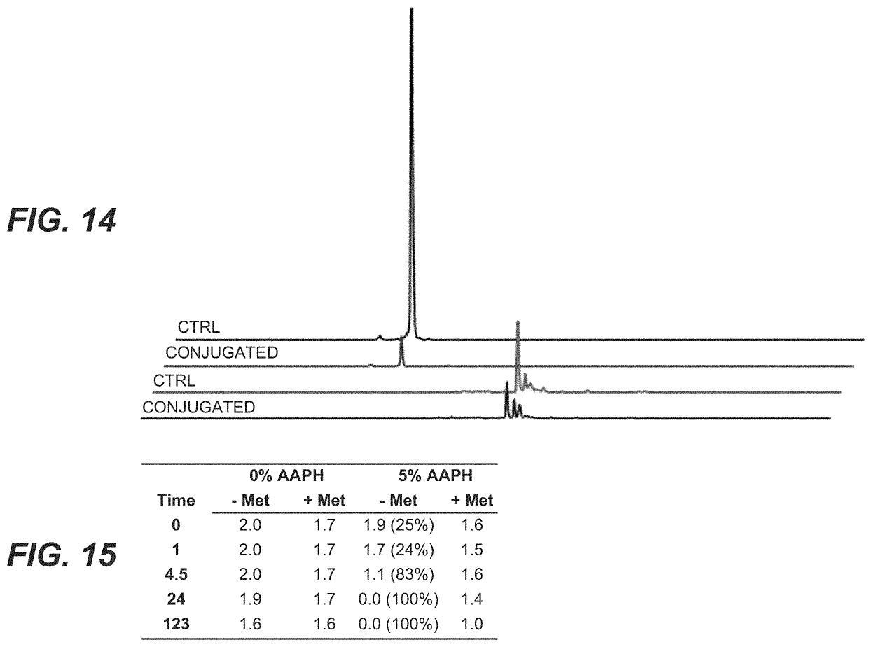

The conjugation site of BPA7 on TMab was characterized via tryptic peptide mapping of the covalent complex using tandem mass spectrometry. Given that benzophenone radicals are known to preferentially react with methionines over other amino acids, Met-252 or Met-428 in the Fc-III peptide binding pocket was likely reacting with the BPA residue of BPA7. A >90% reduction in peak intensity was detected for the tryptic peptide encompassing Met-252, indicative of a reaction with this peptide. Peak intensity for the peptide containing Met-428 was, by comparison, much less affected (FIG. 14). (Dorman, G.; Nakamura, H.; Pulsipher, A.; Prestwich, G. D., The Life of Pi Star: Exploring the Exciting and Forbidden Worlds of the Benzophenone Photophore. Chemical reviews 2016, 116 (24), 15284-15398: Wittelsberger, A.; Thomas, B. E.; Mierke, D. F.; Rosenblatt, M., Methionine acts as a “magnet” in photoaffinity crosslinking experiments. FEBS letters 2006, 580 (7), 1872-1876).

A crystal structure of BPA7 covalently conjugated to the human Fc domain derived from TMab at 2.6 Å was obtained (FIG. 4A). The electron-density omit map encompassing the Bpa residue of BPA7 showed that the carbon between the two phenyl rings of the BPA side chain is tetrahedral, with a (S) stereochemical configuration, and is covalently connected to the epsilon carbon of the Met-252 side chain on the Fc domain. The particular geometry of the complex between BPA7 and the Fc domain appears to drive a highly specific regio- and stereoselective reaction between the two.

The overlay of the original Fc-III peptide bound to the Fc domain onto the BPA7/Fc domain structure showed that the original binding pose of the peptide is largely preserved in the photoconjugate (RMSD less than 0.3 Å for both peptides; FIG. 4B). (DeLano, W. L.; Ultsch, M. H.; de Vos, A. M.; Wells, J. A., Convergent solutions to binding at a protein-protein interface. Science 2000, 287 (5456), 1279-83). On the Fc domain, the side chain of Met-428 must move more than 5.0 Å to accommodate the terminal phenyl ring of the BPA amino acid introduced in place of Val-10 on the peptide (FIG. 4C). This conformation of Met-428 has not been observed in any of the reported structures of the human Fc domain, even in complex with proteins that bind to the same general locale as does Fc-III (e.g., protein A). These results suggested that the conformation of the Met-428 side chain adopted in the BPA7/Fc complex may be intrinsically unfavorable, but that the energetic penalty paid to adopt this conformation is offset by the covalent bond formed between BPA7 and Met-252. Hydrophobic packing or favorable pi-thioether interactions between the BPA phenyl groups and the Met-428 side chain may also help to stabilize the Met-428 conformation. (Valley, C. C.; Cembran, A.; Perlmutter, J. D.; Lewis, A. K.; Labello, N. P.; Gao, J.; Sachs, J. N., The methionine-aromatic motif plays a unique role in stabilizing protein structure. The Journal of biological chemistry 2012, 287 (42), 34979-34991).

Example 8: Influence of Met-252 oxidation or mutations on photocrosslinking. Methionine 252 in the Fc domain of human IgGs is conserved in all human IgG antibody subclasses (IgG1, IgG2, IgG3 and IgG4) and in several antibodies from other species (e.g., rabbit IgG, murine IgG2 and rat IgG2C), although conservation is not universal in IgGs (FIG. 9). Modification of Met-252 can impact circulating antibody half-life in vivo: oxidation to the sulfoxide reduces half-life due to reduced FcRn binding whereas mutation of Met-252 and other residues can lead to increased half-life due to increased FcRn binding (e.g., the so-called “YTE” mutant, which includes the three mutations Met-252→Tyr, Ser-254→Thr, and Thr-256→Glu). (Dall'Acqua, W. F.; Kiener, P. A.; Wu, H., Properties of human IgG1s engineered for enhanced binding to the neonatal Fc receptor (FcRn). J Biol Chem 2006, 281 (33), 23514-24: Gao, X.; Ji, J. A.; Veeravalli, K.; Wang, Y. J.; Zhang, T.; Mcgreevy, W.; Zheng, K.; Kelley, R. F.; Laird, M. W.; Liu, J.; Cromwell, M., Effect of individual Fc methionine oxidation on FcRn binding: Met252 oxidation impairs FcRn binding more profoundly than Met428 oxidation. Journal of pharmaceutical sciences 2015, 104 (2), 368-377). Representative human and non-human monoclonal antibodies, were assessed for the impact of mutational or oxidative changes to Met-252 in the Fc on efficiency of photocrosslinking to BPA7.

Conjugation of BPA7 to another human IgG1 antibody, Rituximab, and a human IgG4 antibody were both effective (DAR=2.0 in both cases). Conjugation of BPA7, resulted in no detectable conjugate with a human IgG4 “YTE” mutant (DAR=0.0; Table 2). Crosslinking to a rabbit IgG was observed (DAR=1.2; antibody “C” in Table 2), but no conjugation was observed to a mouse IgG1 antibody (DAR=0; antibody “D”). These results are consistent with the conclusion that Met-252 is required for effective photoconjugation of peptide BPA7 to the Fc since antibodies lacking Met-252 conjugated. Rabbit IgG has a methionine at the corresponding position 252 and the residues surrounding it are identical to those in human IgG1. Conjugation to a rabbit IgG did not proceed as effectively as to human antibodies. Possibly, subtle conformational differences between human and rabbit mAbs that explains differential binding and/or photoconjugation to peptide BPA7.

TABLE 10
Sequence alignment of human, rabbit and mouse
IgG isotypes showing region surrounding Met-252
(human IgG1 numbering) and associated DARs
reached upon photoconjugation to BPA7.

| Antibody | Species | Subclass | Sequence | DAR |
|---|---|---|---|---|
| Trastuzumab | Human | IgG1 | DTLMISRTPEVTCVVV | 2.0 |
| Rituximab | Human | IgG1 | DTLMISRTPEVTCVVV | 2.0 |
| A | Human | IgG4 | DTLMISRTPEVTCVVV | 2.0 |
| B | Human | IgG4 | DTLYITREPEVTCVVV | 0.0 |
| C | Rabbit | IgG | DTLMISRTPEVTCVVV | 1.2 |
| D | Mouse | IgG1 | DVLTITLTPKVTCVVV | 0.0 |

The impact of oxidation of Met-252 in TMab on conjugation to peptide BPA7 was assessed. Both Met-252 and Met-428 are susceptible to oxidation under certain stress conditions (e.g., elevated temperatures, chemical oxidants, exposure to UV light), which converts the thioether side chain of these residues to a sulfoxide. (Chumsae, C.; Gaza-Bulseco, G.; Sun, J.; Liu, H., Comparison of methionine oxidation in thermal stability and chemically stressed samples of a fully human monoclonal antibody. J Chromatogr B Analyt Technol Biomed Life Sci 2007, 850 (1-2), 285-94: Ji, J. A.; Zhang, B.; Cheng, W.; Wang, Y. J., Methionine, tryptophan, and histidine oxidation in a model protein, PTH: mechanisms and stabilization. J Pharm Sci 2009, 98 (12), 4485-500: Lam, X. M.; Yang, J. Y.; Cleland, J. L., Antioxidants for prevention of methionine oxidation in recombinant monoclonal antibody HER2. J Pharm Sci 1997, 86 (11), 1250-5). To induce methionine oxidation in the Fc, samples were treated with the oxidant 2,2-azobis(2-amidinopropane) dihydrochloride (AAPH) at 37° C. for up to 123 hours. (Ji, J. A.; Zhang, B.; Cheng, W.; Wang, Y. J., Methionine, tryptophan, and histidine oxidation in a model protein, PTH: mechanisms and stabilization. J Pharm Sci 2009, 98 (12), 4485-500). Oxidation of Met-252 by mass spectrometry of the tryptic peptide covering this residue was monitored over time in antibody samples purified from the AAPH reaction followed by photocrosslinking reactions with to BPA7.

A negative correlation between extent of Met-252 oxidation on TMab and extent of crosslinking to BPA7 was observed (FIG. 15A). Since AAPH is a nonspecific oxidant of both Met and Trp, without being bound by any particular theory, it is possible that lack of BPA7 conjugation to AAPH-treated Trastuzumab is not due to Met-252 oxidation alone. It has been shown previously that the addition of free methionine in excess can selectively prevent AAPH-induced oxidation of Met-252 and other methionines in antibodies. (Ji, J. A.; Zhang, B.; Cheng, W.; Wang, Y. J., Methionine, tryptophan, and histidine oxidation in a model protein, PTH: mechanisms and stabilization. J Pharm Sci 2009, 98 (12), 4485-500: Xu, K.; Liu, L.; Saad, 0. M.; Baudys, J.; Williams, L.; Leipold, D.; Shen, B.; Raab, H.; Junutula, J. R.; Kim, A.; Kaur, S., Characterization of intact antibody-drug conjugates from plasma/serum in vivo by affinity capture capillary liquid chromatography-mass spectrometry. Analytical biochemistry 2011, 412 (1), 56-66). TMab treated with 5% AAPH for 24 hours in the presence of excess free methionine demonstrated less oxidation and greater conjugation (FIG. 15B). Oxidation of Met-252 in the Fc domain appears to ablate photoconjugation of BPA7.

BPA7 is highly selective for conjugation to the terminal epsilon carbon of the side chain of Met-252 in the Fc domain of antibodies that bear this residue. Both relatively small modifications of Met-252 (oxidation) and larger modifications (e.g., mutation to Tyr) prevent photoconjugation to BPA7. These data are consistent with findings that benzophenone-based photoaffinity probes preferentially react with methionine residues on their targets. (Wittelsberger, A.; Thomas, B. E.; Mierke, D. F.; Rosenblatt, M., Methionine acts as a “magnet” in photoaffinity crosslinking experiments. FEBS letters 2006, 580 (7), 1872-1876).

Example 9: Application of photoconjugation to construction of site-specific ADCs. To explore the applicability of the photoconjugation reaction to generation of antibody drug conjugates (ADCs), a variant of BPA7 bearing a protected thiol was synthesized. Such a variant enabled, after photoconjugation and deprotection, attachment of thiol-reactive payloads. N-succinimidyl S-acetylthioacetate (SATA) and a PEG-containing SATA variant (SATA-PEG) as the group bearing the protected thiol (attached to the N-terminus) were synthesized to give SATA-BPA7 and SATA-PEG-BPA7, respectively (FIG. 5A). Both of these peptides were photoconjugated to TMab, the conjugates were purified, the SATA acetyl groups removed with hydroxylamine and the conjugates were stored as free thiols available for conjugation to payloads.

Both SATA-BPA7 and SATA-PEG-BPA7 antibody conjugates were formed and deprotected efficiently as indicated by LCMS (FIG. 5B). The SATA-BPA7/TMab conjugate aggregated upon extended storage at 4 degrees C., as indicated by size-exclusion chromatography (FIG. 16). These results are consistent with previous reports highlighting the solubility-enhancing effects of PEG groups on ADCs. (King, H. D.; Dubowchik, G. M.; Mastalerz, H.; Willner, D.; Hofstead, S. J.; Firestone, R. A.; Lasch, S. J.; Trail, P. A., Monoclonal antibody conjugates of doxorubicin prepared with branched peptide linkers: inhibition of aggregation by methoxytriethyleneglycol chains. Journal of medicinal chemistry 2002, 45 (19), 4336-4343: Miller, M. L.; Roller, E. E.; Zhao, R. Y.; Leece, B. A.; Ab, O.; Baloglu, E.; Goldmacher, V. S.; Chari, R. V. J., Synthesis of taxoids with improved cytotoxicity and solubility for use in tumor-specific delivery. Journal of medicinal chemistry 2004, 47 (20), 4802-4805: Moon, S.-J.; Govindan, S. V.; Cardillo, T. M.; D'Souza, C. A.; Hansen, H. J.; Goldenberg, D. M., Antibody conjugates of 7-ethyl-10-hydroxycamptothecin (SN-38) for targeted cancer chemotherapy. Journal of medicinal chemistry 2008, 51 (21), 6916-6926). While freezing either conjugate at −80 degrees C. prevented aggregation, further studies proceeded with the use of the SATA-PEG-BPA7 conjugate. The free thiols of TMab/SATA-PEG-BPA7 were reacted with ε-maleimido-caproyl-valine-citrulline-para-aminobenzyl-monomethyl auristatin E (mc-vc-PAB-MMAE) and the conjugate was purified. The resulting ADC had a final DAR of 1.9, corresponding to final number of MMAE moieties attached to the antibody, and was 94.7% monomeric by SEC (FIG. 5D).

The cytotoxicity of the TMab/SATA-PEG-BPA7/MMAE conjugate and a THIOMAB™ antibody drug conjugate (TDC) bearing the same payload (DAR=1.9) was measured in Her2-expressing cell lines KPL-4 and SK-BR3 (FIG. 6). Potency as measured by IC₅₀ value for the photoconjugate was equivalent to that of the TDC (e.g., 1.7 versus 2.0 ng/mL in Sk-BR-3 cells) indicating that binding, internalization and release of the cytotoxic MMAE payload was likely unaffected by the photoconjugation format versus the more conventional TDC format.

The stability of the TMab/SATA-PEG-BPA7/MMAE conjugate was measured in plasma from rats, cynomolgus monkeys and humans (FIG. 7). Over 96 hours of incubation, minimal degradation or deconjugation of the payload from the photoconjugate was observed. The stability of the photoconjugate was comparable to that of a THIOMAB™ antibody/MMAE conjugate employing the LC K149C conjugation site, which we have shown previously gives rise to highly stable thiosuccinimide-linked TDCs in vivo. (Ohri, R.; Bhakta, S.; Fourie-O'Donohue, A.; dela Cruz-Chuh, J.; Tsai, S. P.; Cook, R.; Wei, B.; Ng, C.; Wong, A. W.; Bos, A. B.; Farahi, F.; Bhakta, J.; Pillow, T. H.; Raab, H.; Vandlen, R.; Polakis, P.; Liu, Y.; Erickson, H.; Junutula, J. R.; Kozak, K. R., High-Throughput Cysteine Scanning To Identify Stable Antibody Conjugation Sites for Maleimide- and Disulfide-Based Linkers. Bioconjugate chemistry 2018, 29 (2), 473-485).

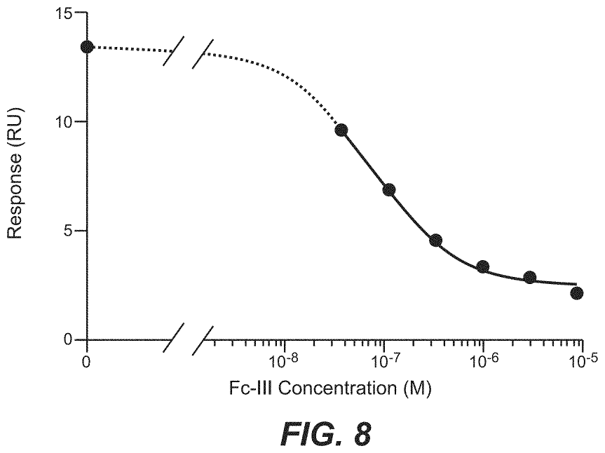

Binding to FcRn is useful for maintaining high circulating half-life of antibodies in vivo, a feature which is usually, but not always desired in therapeutic or imaging applications of antibodies. (Roopenian, D. C.; Akilesh, S., FcRn: the neonatal Fc receptor comes of age. Nature reviews. Immunology 2007, 7 (9), 715-725). Using a competition binding SPR assay, a decrease in FcRn binding to TMab was observed upon increasing concentrations of Fc-III (IC50˜75 nM; FIG. 8). BPA7 occupies the same site as Fc-III, making it possible that FcRn binding would be disrupted in the photoconjugate.

The antibodies and methods herein possess significant advantages relative to photoconjugation methods employing domains from protein A or protein G. For example, the BPA peptides described herein are only 13 residues long and can therefore be readily made and modified via solid-phase peptide synthesis. Incorporation of conjugation handles into Fc-III for the attachment of any payload or label is, in principle, possible with our approach. By contrast, even BPA-containing peptides derived from domains from protein A or protein G, which can be photoconjugated efficiently to antibodies are ˜60 residues in length and difficult to generate or modify synthetically. (Hui, J. Z.; Al Zaki, A.; Cheng, Z.; Popik, V.; Zhang, H.; Luning Prak, E. T.; Tsourkas, A., Facile method for the site-specific, covalent attachment of full-length IgG onto nanoparticles. Small (Weinheim an der Bergstrasse, Germany) 2014, 10 (16), 3354-3363: Hui, J. Z.; Tsourkas, A., Optimization of Photoactive Protein Z for Fast and Efficient Site-Specific Conjugation of Native IgG. Bioconjugate chemistry 2014, 25 (9), 1709-1719). The shorter length of the Fc-III-derived photoconjugation peptides described herein also likely lower immunogenicity in vivo relative to the reagents based on domains from protein A or protein G, both of which are bacterial in origin. A recent report highlighted the use of the Fc-III peptide containing a Bpa residue in generating immunotoxins and recapitulates, albeit at lower resolution, our finding that replacement of the valine in the Fc-III sequence with Bpa results in an effective crosslink to Met-252 in the Fc domain. (Park, J.; Lee, Y.; Ko, B. J.; Yoo, T. H., Peptide-Directed Photo-Cross-Linking for Site-Specific Conjugation of IgG. Bioconjugate chemistry 2018). However, like studies employing photoaffinity reagents based on protein A and G, that study employed recombinantly-expressed Fc-III fusion proteins incorporating the non-natural Bpa residue. Thus, other photoaffinity ligands known in the art are disadvantaged comparably to the BPA peptides herein because the BPA peptides are better accessible via chemical synthesis and have reduced size—features absent from known photoaffinity ligands.

The photoconjugation methods described herein allow for the facile generation of homogeneous antibody conjugates for various biological applications. As a prelude to such studies, we demonstrated functional activity in cells of a cytotoxic ADC generated from the photoconjugation method and showed that in plasma the photoconjugate was completely stable for at least 5 days, a finding that portends well for stability in vivo.

Applications of the antibodies and methods described herein include radioactivity-based immunotherapy or imaging for which long circulating half-lives can, in both cases, increase radiation-induced toxicity and, in the latter case, reduce image contrast. (Jaggi, J. S.; Carrasquillo, J. A.; Seshan, S. V.; Zanzonico, P.; Henke, E.; Nagel, A.; Schwartz, J.; Beattie, B.; Kappel, B. J.; Chattopadhyay, D.; Xiao, J.; Sgouros, G.; Larson, S. M.; Scheinberg, D. A., Improved tumor imaging and therapy via i.v. IgG-mediated time-sequential modulation of neonatal Fc receptor. Journal of Clinical Investigation 2007, 117 (9), 2422-2430). For ocular applications of antibody therapeutics, FcRn binding can be detrimental as it drives clearance from the eye, providing another potential area where the photoconjugation methods herein may be employed. (Kim, H.; Robinson, S. B.; Csaky, K. G., FcRn receptor-mediated pharmacokinetics of therapeutic IgG in the eye. Molecular vision 2009, 15, 2803-2812). Lastly, the photoconjugation method described herein may be useful in a variety of in vitro applications that would benefit from site-specific conjugation to wild-type antibodies. For example, the developed photoconjugation reaction herein uses 96-well plates with relatively small antibody amounts (˜0.4 mg), making it possible to generate libraries of homogeneously-labeled antibody conjugates from hybridomas provided the host species produces antibodies with Met-252 (e.g., rabbit). This capability could be useful for enabling more robust comparison of antibody clones for binding, internalization or potency studies, a process that would otherwise involve individual expression and purification of antibody mutants for conjugation. (Ohri, R.; Bhakta, S.; Fourie-O'Donohue, A.; dela Cruz-Chuh, J.; Tsai, S. P.; Cook, R.; Wei, B.; Ng, C.; Wong, A. W.; Bos, A. B.; Farahi, F.; Bhakta, J.; Pillow, T. H.; Raab, H.; Vandlen, R.; Polakis, P.; Liu, Y.; Erickson, H.; Junutula, J. R.; Kozak, K. R., High-Throughput Cysteine Scanning To Identify Stable Antibody Conjugation Sites for Maleimide- and Disulfide-Based Linkers. Bioconjugate chemistry 2018, 29 (2), 473-485: Catcott, K. C.; McShea, M. A.; Bialucha, C. U.; Miller, K. L.; Hicks, S. W.; Saxena, P.; Gesner, T. G.; Woldegiorgis, M.; Lewis, M. E.; Bai, C.; Fleming, M. S.; Ettenberg, S. A.; Erickson, H. K.; Yoder, N. C., Microscale screening of antibody libraries as maytansinoid antibody-drug conjugates. mAbs 2016, 8 (3), 513-523: Puthenveetil, S.; Musto, S.; Loganzo, F.; Tumey, L. N.; O&apos; Donnell, C. J.; Graziani, E. I., Development of solid-phase site-specific conjugation and its application towards generation of dual labeled antibody and Fab drug conjugates. Bioconjugate chemistry 2016, acs.bioconjchem.6b00054: Nath, N.; Godat, B.; Benink, H.; Urh, M., On-bead antibody-small molecule conjugation using high-capacity magnetic beads. Journal of immunological methods 2015)

Although the foregoing invention has been described in some detail by way of illustration and example for purposes of clarity of understanding, the descriptions and examples should not be construed as limiting the scope of the invention. Accordingly, all suitable modifications and equivalents may be considered to fall within the scope of the invention as defined by the claims that follow. The disclosures of all patent and scientific literature cited herein are expressly incorporated in their entirety by reference.

1. A BPA peptide composition comprising a peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11.

2. The BPA peptide composition of claim 1, wherein the BPA peptide is BPA7 (SEQ ID NO:8).

3. The BPA peptide composition of claim 1, wherein the BPA peptide is BPA10 (SEQ ID NO:11).

4. The BPA peptide composition of claim 1, wherein the BPA peptide is BPA 3 (SEQ ID NO:4) or BPA4 (SEQ ID NO:5)

5. A PhL peptide composition comprising a peptide comprising SEQ ID NO:12, SEQ ID NO:13, SEQ ID NO:14, SEQ ID NO:14, SEQ ID NO:15, SEQ ID NO:16, SEQ ID NO:17, SEQ ID NO:18, or SEQ ID NO:19, SEQ ID NO:20.

6. A Tdf peptide composition comprising a peptide comprising SEQ ID NO:21, SEQ ID NO:22, SEQ ID NO:23, SEQ ID NO:24, SEQ ID NO:25, SEQ ID NO:26, SEQ ID NO:27, SEQ ID NO:28, or SEQ ID NO:29.

7. An antibody-drug conjugate comprising
(i) an antibody; and
(ii) a BPA peptide of claim 1 covalently attached in the Fc portion of the antibody.

8. The antibody-drug conjugate composition of claim 7 having Formula (I):
Ab [symbol] B-E-L-D)ₚ (I)
wherein:
Ab is an antibody;
B is a BPA peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11 covalently attached to the Fc region of the antibody and to L;
E is an optional extension moiety as provided herein;
L is a linker moiety;
D is a drug moiety comprising a radiolabel, an antibody, or an anti-cancer agent such as a tubulin inhibitor, a topoisomerase II inhibitor, a DNA crosslinking cytoxic agent, an alkylating agent, a taxane, or an anthracycline agent; and
p is 1 or 2.

9. The antibody-drug conjugate composition of claim 8 comprising a homogenous mixture of antibody-drug conjugates wherein p is 2.

10. The antibody-drug conjugate composition of claim 8, wherein the antibody is a monoclonal, IgG antibody.

11. The antibody-drug conjugate composition of claim 10 wherein the antibody is a cysteine-engineered antibody.

12. The antibody-drug conjugate of claim 10, wherein Ab is trastuzumab or trastuzumab emtansine.

13. The antibody-drug conjugate of claim 8, wherein D is a maytansinoid, dolastatin, auristatin, calicheamicin, pyrrolobenzodiazepine dimer (PBD dimer), an anthracycline agent, duocarmycin, a synthetic duocarmycin analogue, a 1,2,9,9a-Tetrahydrocyclopropa[c]benzo[e]indol-4-one (CBI) dimer, a vinca alkaloid, a taxane (e.g. paclitaxel or docetaxel), trichothecene, camptothecin, silvestrol, or elinafide.

14. The antibody-drug conjugate of claim 13, wherein D is a duocarmycin comprising mycarosylprotylonolide.

15. The antibody-drug conjugate of claim 13, wherein D is a PBD dimer.

16. The antibody-drug conjugate of claim 13, wherein D is a CBI dimer.

17. The antibody-drug conjugate of claim 13, wherein D is an auristatin comprising MMAE or MMAF.

18. The antibody-drug conjugate of claim 13, wherein D is an anthracycline agent comprising PNU-159682, doxorubicin, daunorubicin, epirubicin, idarubicin, mitoxantrone, or valrubicin.

19. The antibody-drug conjugate of claim 13, wherein D is conjugated to a radiolabel.

20. The antibody-drug conjugate of claim 19, wherein the radiolabel is ¹¹C, ¹³N, ¹⁵O, ¹⁸F, ³²P, ⁵¹Cr, ⁵⁷Co, ⁶⁴Cu, ⁶⁷Ga, ⁷⁵Se, ^{81m}Kr, ⁸²Rb, ^{99m}Tc, ¹²³I, ¹²⁵I, ¹³¹I, ¹¹¹In, or ²⁰¹Ti.

21. The antibody-drug conjugate of claim 8, wherein L comprises formula (IV):
-Str-(Pep)ₘ(Y)ₙ- (IV)
wherein,
Str is a stretcher unit or S covalently attached the BPA peptide;
Pep is an optional peptide unit of two to twelve amino acid residues;
Y is an optional spacer unit covalently attached to D; and
m and n are independently selected from 0 and 1.

22. The antibody conjugation of claim 21, wherein Str comprises a maleimidyl, bromacetamidyl or iodoacetamidyl moiety.

23. The antibody conjugation of claim 21, wherein Str has the formula (V): 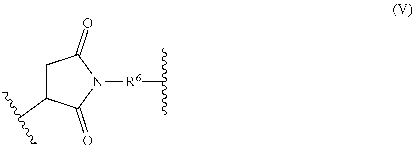
wherein,
R⁶ comprises C₁-C₁₂ alkylene, C₁-C₁₂ alkylene-C(═O), C₁-C₁₂ alkylene-NH, (CH₂CH₂O)ᵣ, (CH₂CH₂O)ᵣ—C(═O), (CH₂CH₂O)ᵣ—CH₂, or C₁-C₁₂ alkylene-NHC(═O)CH₂CH (thiophen-3-yl);
r is an integer ranging from 1 to 12; and
R⁶ is attached to Pep or Y.

24. The antibody-drug conjugate of claim 21, wherein pep comprises a peptidomimetic moiety comprising:

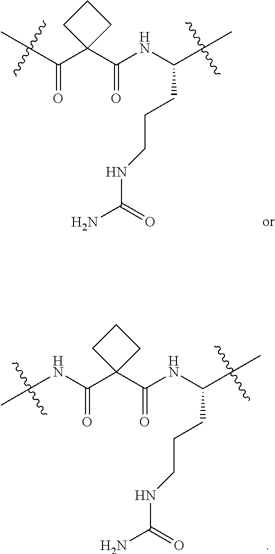 [SMILES: *C(=O)C1(C(=O)N[C@H](*)CCCNC(N)=O)CCC1 ; *NC(=O)C1(C(=O)N[C@H](*)CCCNC(N)=O)CCC1]

25. The antibody-drug conjugate of claim 21, wherein, L comprises formula (IV) where R₆ is (CH₂)₅, Pep is val-cit, sq-cit, or nsq-cit, and Y is p-aminobenzyloxycarbonyl (PAB).

26. The antibody-drug conjugate claim 8, wherein L comprises the formula (VI): 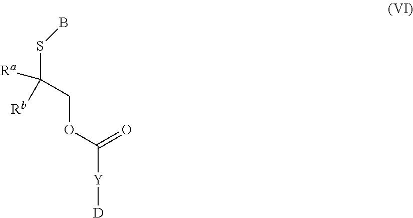
wherein,
B is a BPA peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11 covalently attached to the Fc region of the antibody and to L;
Y is para-aminobenzyl, p-aminobenzyloxycarbonyl (PAB), 2-aminoimidazol-5-methanol derivatives, ortho- or para-aminobenzylacetals, 4-aminobutyric acid amides, bicyclo[2.2.1] and bicyclo[2.2.2] ring systems, or 2-aminophenylpropionic acid amides; and
R^{a} and R^{b} are independently selected from H and C₁₋₃ alkyl, wherein only one of R^{a} and R^{b} can be H, or R^{a} and R^{b} together with the carbon atom to which they are bound form a four- to six-membered ring optionally comprising an oxygen heteroatom

27. The antibody-drug conjugate of claim 26, wherein Y is para-aminobenzyl or p-aminobenzyloxycarbonyl.

28. The antibody-drug conjugate of claim 8, wherein,
B is BPA7 (SEQ ID NO:8);
Ab is Trastuzumab;
D is MMAE or MMAF; and
L comprises a compound of formula (IV):
-Str-(Pep)ₘ-(Y)ₙ- (IV)
wherein Str is a compound of formula (V): 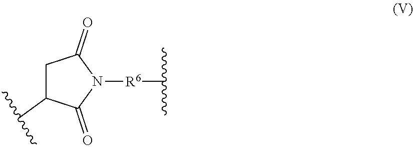
wherein, R₆ is (CH₂)₅,
Pep is val-cit, sq-cit, or nsq-cit; and
Y is p-aminobenzyloxycarbonyl (PAB).

29. The antibody-drug conjugate of claim 28, wherein the antibody binds to a tumor-associated antigen or cell-surface receptor.

30. The antibody-drug conjugate of claim 29, wherein the tumor-associated antigen or cell-surface receptor is selected from the group consisting of (1)-(53):
(1) BMPR1B (bone morphogenetic protein receptor-type IB);
(2) E16 (LAT1, SLC7A5);
(3) STEAP1 (six transmembrane epithelial antigen of prostate);
(4) MUC16 (0772P, CA125);
(5) MPF (MPF, MSLN, SMR, megakaryocyte potentiating factor, mesothelin);
(6) Napi2b (NAPI-3B, NPTIIb, SLC34A2, solute carrier family 34 (sodium phosphate), member 2, type II sodium-dependent phosphate transporter 3b);
(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B);
(8) PSCA hlg (2700050C12Rik, C530008O16Rik, RIKEN cDNA 2700050C12, RIKEN cDNA 2700050C12 gene);
(9) ETBR (Endothelin type B receptor);
(10) MSG783 (RNF124, hypothetical protein FLJ20315);
(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein);
(12) TrpM4 (BR22450, FLJ20041, TRPM4, TRPM4B, transient receptor potential cation channel, subfamily M, member 4);
(13) CRIPTO (CR, CR1, CRGF, CRIPTO, TDGF1, teratocarcinoma-derived growth factor);
(14) CD21 (CR2 (Complement receptor 2) or C3DR (C3d/Epstein Barr virus receptor) or Hs 73792);
(15) CD79b (CD79B, CD79β, IGb (immunoglobulin-associated beta), B29);
(16) FcRH2 (IFGP4, IRTA4, SPAP1 Å (SH2 domain containing phosphatase anchor protein 1a), SPAP1B, SPAP1C);
(17) HER2;
(18) NCA;
(19) MDP;
(20) IL20Rα;
(21) Brevican;
(22) EphB2R;
(23) ASLG659;
(24) PSCA;
(25) GEDA;
(26) BAFF-R (B cell-activating factor receptor, BLyS receptor 3, BR3);
(27) CD22 (B-cell receptor CD22-B isoform);
(28) CD79a (CD79A, CD79α, immunoglobulin-associated alpha);
(29) CXCR5 (Burkitt's lymphoma receptor 1);
(30) HLA-DOB (Beta subunit of MHC class II molecule (Ia antigen));
(31) P2X5 (Purinergic receptor P2X ligand-gated ion channel 5);
(32) CD72 (B-cell differentiation antigen CD72, Lyb-2);
(33) LY64 (Lymphocyte antigen 64 (RP105), type I membrane protein of the leucine rich repeat (LRR) family);
(34) FcRH1 (Fc receptor-like protein 1);
(35) FcRH5 (IRTA2, Immunoglobulin superfamily receptor translocation associated 2);
(36) TENB2 (putative transmembrane proteoglycan);
(37) PMEL17 (silver homolog; SILV; D12S53E; PMEL17; SI; SIL);
(38) TMEFF1 (transmembrane protein with EGF-like and two follistatin-like domains 1; Tomoregulin-1);
(39) GDNF-Ra1 (GDNF family receptor alpha 1; GFRA1; GDNFR; GDNFRA; RETL1; TRNR1; RET1L; GDNFR-alpha1; GFR-ALPHA-1);
(40) Ly6E (lymphocyte antigen 6 complex, locus E; Ly67, RIG-E, SCA-2,TSA-1);
(41) TMEM46 (shisa homolog 2 (Xenopus laevis); SHISA2);
(42) Ly6G6D (lymphocyte antigen 6 complex, locus G6D; Ly6-D, MEGT1);
(43) LGR5 (leucine-rich repeat-containing G protein-coupled receptor 5; GPR49, GPR67);
(44) RET (ret proto-oncogene; MEN2A; HSCR1; MEN2B; MTC1; PTC; CDHF12; Hs.168114; RET51; RET-ELE1);
(45) LY6K (lymphocyte antigen 6 complex, locus K; LY6K; HSJ001348; FLJ35226);
(46) GPR19 (G protein-coupled receptor 19; Mm.4787);
(47) GPR54 (KISS1 receptor; KISS1R; GPR54; HOT7T175; AXOR12);
(48) ASPHD1 (aspartate beta-hydroxylase domain containing 1; LOC253982);
(49) Tyrosinase (TYR; OCAIA; OCA1A; tyrosinase; SHEP3);
(50) TMEM118 (ring finger protein, transmembrane 2; RNFT2; FLJ14627);
(51) GPR172A (G protein-coupled receptor 172A; GPCR41; FLJ11856; D15Ertd747e);
(52) CD33; and
(53) CLL-1.

31. A pharmaceutical composition comprising the antibody-drug conjugate composition according to claim 8 and a pharmaceutically acceptable excipient.

32. A method of treating lung cancer, bladder cancer, renal cell cancer (RCC), melanoma, or breast cancer, the method comprising administering to said patient an effective amount of an antibody-drug conjugate of claim 8.

33. A method of treating breast cancer, the method comprising administering to a patient having said breast cancer an effective amount of an antibody-drug conjugate of claim 8.

34. A method of treating lung cancer, the method comprising administering to a patient having said lung cancer an effective amount of an antibody-drug conjugate of claim 8.

35. The method of claim 34, wherein the lung cancer is non-small cell lung cancer.

36. A method of treating bladder cancer, the method comprising administering to a patient having said bladder cancer an effective amount of an antibody-drug conjugate of claim 8.

37. A method of treating kidney cancer, the method comprising administering to a patient having said kidney cancer an effective amount of an antibody-drug conjugate of claim 8.

38. The method of claim 32, wherein the antibody-drug conjugate is co-administered with another anticancer agent.

39. The method of claim 38, wherein the anticancer agent comprises one or more therapeutic antibodies.

40. The method of claim 38, wherein the anticancer agent is radiation therapy or chemotherapy.

41. A method of imaging a patient for a tumor, the method comprising administering to the patient a composition comprising an ADC of claim 8 and detecting the quantity and location of the label.

42. The method of claim 41, wherein the label comprises ¹¹C, ¹³N, ¹⁵O, ¹⁸F, ³²P, ⁵¹Cr, ⁵⁷O, ⁶⁴Cu, ⁶⁷Ga, ⁷⁵Se, ^{81m}Kr, ⁸²Rb, ^{99m}TC, ¹²³I, ¹²⁵I, ¹³¹I, ¹¹¹In, or ²⁰¹Ti.

43. A method to prepare an antibody-drug conjugate composition of claim 8, the method comprising:
(i) reacting an antibody under photo-crosslinking conditions with a BPA peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11 thereby forming an antibody conjugate;
(ii) optionally removing a protecting group on the terminal end of the BPA peptide;
(iii) reacting the antibody conjugate with a drug (D) further comprising a linker to form the antibody-drug conjugate composition having Formula (I), wherein the linker comprises formula (IV):
-Str-(Pep)ₘ-(Y)ₙ- (IV)
wherein,
Str is a stretcher unit or S covalently attached the BPA peptide;
Pep is an optional peptide unit of two to twelve amino acid residues;
Y is an optional spacer unit covalently attached to D; and
m and n are independently selected from 0 and 1.

44. The method of claim 43, wherein the antibody is a monoclonal, IgG antibody.

45. The method of claim 43, wherein the antibody is a cysteine-engineered antibody.

46. The method of any one of claims 43-45, wherein the antibody binds to a tumor-associated antigen or cell-surface receptor.

47. The method claim 43, wherein the BPA peptide is BPA7 (SEQ ID NO:8).

48. The method of claim 47, wherein the BPA peptide further comprises an extension moiety comprising PEG.

49. The method of claim 48, wherein the extension moiety is PEG₁₂-SATA or SATA.

50. The method of claim 43, wherein photo-crosslinking conditions comprise irradiating under ultraviolet (UV) light.

51. The method of claim 43, wherein the antibody and the BPA peptide are irradiated with 365 nm UV light.

52. The method of claim 43, wherein the photo-crosslinking conditions comprise irradiating the antibody and the BPA peptide in a multi-well plate.

53. The method of claim 43, wherein photo-crosslinking conditions further comprise an antioxidant.

54. The method of claim 53, wherein the antioxidant is selected from the group consisting of 5-hydroxyindole (5-HI), methionine, sodium thiosulfate, catalase, platinum, tryptophan, 5-methoxy-tryptophan, 5-amino-tryptophan, 5-fluoro-tryptophan, N-acetyl tryptophan, tryptamine, tryptophanamide, serotonin, melatonin, kynurenine, indolyl derivatives, salicylic acid, 5-hydroxy salicylic acid, anthranilic acid, and 5-hydroxy anthranilic acid.

55. A method to prepare an antibody-drug conjugate composition of claim 8, the method comprising reacting an antibody under photo-crosslinking conditions with a BPA peptide comprising SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11, wherein the BPA peptide is covalently attached to a drug moiety (D) through a linker comprising formula (IV):
-Str-(Pep)ₘ-(Y)ₙ- (IV)
wherein,
Str is a stretcher unit or S covalently attached the BPA peptide;
Pep is an optional peptide unit of two to twelve amino acid residues;
Y is an optional spacer unit covalently attached to D; and
m and n are independently selected from 0 and 1,
thereby forming an antibody conjugate.

56. The method of claim 55, wherein the antibody is a monoclonal, IgG antibody.

57. The method of claim 55, wherein the antibody is a cysteine-engineered antibody.

58. The method of any one of claims 55-57, wherein the antibody binds to a tumor-associated antigen or cell-surface receptor.

59. The method of claim 55, wherein the BPA peptide is BPA7 (SEQ ID NO:8).

60. The method of claim 55, wherein the BPA peptide further comprises an extension moiety comprising PEG.

61. The method of claim 60, wherein the extension moiety is PEG₁₂-SATA or SATA.

62. The method of claim 55, wherein photo-crosslinking conditions comprise irradiating under ultraviolet (UV) light.

63. The method of claim 55, wherein the antibody and the BPA peptide are irradiated with 365 nm UV light.

64. The method of claim 55, wherein the photo-crosslinking conditions comprise irradiating the antibody and the BPA peptide in a multi-well plate.

65. The method of claim 55, wherein photo-crosslinking conditions further comprise an antioxidant.

66. The method of claim 65, wherein the antioxidant is selected from the group consisting of 5-hydroxyindole (5-HI), methionine, sodium thiosulfate, catalase, platinum, tryptophan, 5-methoxy-tryptophan, 5-amino-tryptophan, 5-fluoro-tryptophan, N-acetyl tryptophan, tryptamine, tryptophanamide, serotonin, melatonin, kynurenine, indolyl derivatives, salicylic acid, 5-hydroxy salicylic acid, anthranilic acid, and 5-hydroxy anthranilic acid.


SEQUENCE LISTING (35 sequences)

SEQ ID NO: 1 - protein, 13 aa, Artificial Sequence
  source: /note=“Description of Artificial Sequence Synthetic peptide”
  source: /note=“N-term Ac”
  source: /note=“C-term NH2”
DCAWHLGELVWCT

SEQ ID NO: 2 - protein, 13 aa, Artificial Sequence
  source: /note=“Description of Artificial Sequence Synthetic peptide”
  source: /note=“N-term Ac”
  MOD_RES (1)..(1): p-benzoyl-L-phenylalanine
  source: /note=“C-term NH2”
XCAWHLGELVWCT

SEQ ID NO: 3 - protein, 13 aa, Artificial Sequence
  source: /note=“Description of Artificial Sequence Synthetic peptide”
  source: /note=“N-term Ac”
  MOD_RES (3)..(3): p-benzoyl-L-phenylalanine
  source: /note=“C-term NH2”
DCXWHLGELVWCT

SEQ ID NO: 4 - protein, 13 aa, Artificial Sequence
  source: /note=“Description of Artificial Sequence Synthetic peptide”
  source: /note=“N-term Ac”
  MOD_RES (5)..(5): p-benzoyl-L-phenylalanine
  source: /note=“C-term NH2”
DCAWXLGELVWCT

SEQ ID NO: 5 - protein, 13 aa, Artificial Sequence
  source: /note=“Description of Artificial Sequence Synthetic peptide”
  source: /note=“N-term Ac”
  MOD_RES (6)..(6): p-benzoyl-L-phenylalanine
  source: /note=“C-term NH2”
DCAWHXGELVWCT

SEQ ID NO: 6 - protein, 13 aa, Artificial Sequence
  source: /note=“Description of Artificial Sequence Synthetic peptide”
  source: /note=“N-term Ac”
  MOD_RES (8)..(8): p-benzoyl-L-phenylalanine
  source: /note=“C-term NH2”
DCAWHLGXLVWCT

SEQ ID NO: 7 - protein, 13 aa, Artificial Sequence
  source: /note=“Description of Artificial Sequence Synthetic peptide”
  source: /note=“N-term Ac”
  MOD_RES (9)..(9): p-benzoyl-L-phenylalanine
  source: /note=“C-term NH2”
DCAWHLGEXVWCT

SEQ ID NO: 8 - protein, 13 aa, Artificial Sequence
  source: /note=“Description of Artificial Sequence Synthetic peptide”
  source: /note=“N-term Ac”
  MOD_RES (10)..(10): p-benzoyl-L-phenylalanine
  source: /note=“C-term NH2”
DCAWHLGELXWCT

SEQ ID NO: 9 - protein, 13 aa, Artificial Sequence
  source: /note=“Description of Artificial Sequence Synthetic peptide”
  source: /note=“N-term Ac”
  MOD_RES (11)..(11): p-benzoyl-L-phenylalanine
  source: /note=“C-term NH2”
DCAWHLGELVXCT

SEQ ID NO: 10 - protein, 13 aa, Artificial Sequence
  source: /note=“Description of Artificial Sequence Synthetic peptide”
  source: /note=“N-term Ac”
  MOD_RES (13)..(13): p-benzoyl-L-phenylalanine
  source: /note=“C-term NH2”
DCAWHLGELVWCX

SEQ ID NO: 11 - protein, 15 aa, Artificial Sequence
  source: /note=“Description of Artificial Sequence Synthetic peptide”
  source: /note=“N-term Ac”
  MOD_RES (11)..(11): p-benzoyl-L-phenylalanine
  source: /note=“C-term NH2”
CDCAWHLGELXWCTC

SEQ ID NO: 12 - protein, 13 aa, Artificial Sequence
  source: /note=“Description of Artificial Sequence Synthetic peptide”
  source: /note=“N-term Ac”
  MOD_RES (1)..(1): Diazirinyl leucine
  source: /note=“C-term NH2”
XCAWHLGELVWCT

SEQ ID NO: 13 - protein, 13 aa, Artificial Sequence
  source: /note=“Description of Artificial Sequence Synthetic peptide”
  source: /note=“N-term Ac”
  MOD_RES (3)..(3): Diazirinyl leucine
  source: /note=“C-term NH2”
DCXWHLGELVWCT

SEQ ID NO: 14 - protein, 13 aa, Artificial Sequence
  source: /note=“Description of Artificial Sequence Synthetic peptide”
  source: /note=“N-term Ac”
  MOD_RES (5)..(5): Diazirinyl leucine
  source: /note=“C-term NH2”
DCAWXLGELVWCT

SEQ ID NO: 15 - protein, 13 aa, Artificial Sequence
  source: /note=“Description of Artificial Sequence Synthetic peptide”
  source: /note=“N-term Ac”
  MOD_RES (6)..(6): Diazirinyl leucine
  source: /note=“C-term NH2”
DCAWHXGELVWCT

SEQ ID NO: 16 - protein, 13 aa, Artificial Sequence
  source: /note=“Description of Artificial Sequence Synthetic peptide”
  source: /note=“N-term Ac”
  MOD_RES (8)..(8): Diazirinyl leucine
  source: /note=“C-term NH2”
DCAWHLGXLVWCT

SEQ ID NO: 17 - protein, 13 aa, Artificial Sequence
  source: /note=“Description of Artificial Sequence Synthetic peptide”
  source: /note=“N-term Ac”
  MOD_RES (9)..(9): Diazirinyl leucine
  source: /note=“C-term NH2”
DCAWHLGEXVWCT

SEQ ID NO: 18 - protein, 13 aa, Artificial Sequence
  source: /note=“Description of Artificial Sequence Synthetic peptide”
  source: /note=“N-term Ac”
  MOD_RES (10)..(10): Diazirinyl leucine
  source: /note=“C-term NH2”
DCAWHLGELXWCT

SEQ ID NO: 19 - protein, 13 aa, Artificial Sequence
  source: /note=“Description of Artificial Sequence Synthetic peptide”
  source: /note=“N-term Ac”
  MOD_RES (11)..(11): Diazirinyl leucine
  source: /note=“C-term NH2”
DCAWHLGELVXCT

SEQ ID NO: 20 - protein, 13 aa, Artificial Sequence
  source: /note=“Description of Artificial Sequence Synthetic peptide”
  source: /note=“N-term Ac”
  MOD_RES (13)..(13): Diazirinyl leucine
  source: /note=“C-term NH2”
DCAWHLGELVWCX

SEQ ID NO: 21 - protein, 13 aa, Artificial Sequence
  source: /note=“Description of Artificial Sequence Synthetic peptide”
  source: /note=“N-term Ac”
  MOD_RES (1)..(1): 3-trifluoromethyl-3-phenyldiazarine
  source: /note=“C-term NH2”
XCAWHLGELVWCT

SEQ ID NO: 22 - protein, 13 aa, Artificial Sequence
  source: /note=“Description of Artificial Sequence Synthetic peptide”
  source: /note=“N-term Ac”
  MOD_RES (3)..(3): 3-trifluoromethyl-3-phenyldiazarine
  source: /note=“C-term NH2”
DCXWHLGELVWCT

SEQ ID NO: 23 - protein, 13 aa, Artificial Sequence
  source: /note=“Description of Artificial Sequence Synthetic peptide”
  source: /note=“N-term Ac”
  MOD_RES (5)..(5): 3-trifluoromethyl-3-phenyldiazarine
  source: /note=“C-term NH2”
DCAWXLGELVWCT

SEQ ID NO: 24 - protein, 13 aa, Artificial Sequence
  source: /note=“Description of Artificial Sequence Synthetic peptide”
  source: /note=“N-term Ac”
  MOD_RES (6)..(6): 3-trifluoromethyl-3-phenyldiazarine
  source: /note=“C-term NH2”
DCAWHXGELVWCT

SEQ ID NO: 25 - protein, 13 aa, Artificial Sequence
  source: /note=“Description of Artificial Sequence Synthetic peptide”
  source: /note=“N-term Ac”
  MOD_RES (8)..(8): 3-trifluoromethyl-3-phenyldiazarine
  source: /note=“C-term NH2”
DCAWHLGXLVWCT

SEQ ID NO: 26 - protein, 13 aa, Artificial Sequence
  source: /note=“Description of Artificial Sequence Synthetic peptide”
  source: /note=“N-term Ac”
  MOD_RES (9)..(9): 3-trifluoromethyl-3-phenyldiazarine
  source: /note=“C-term NH2”
DCAWHLGEXVWCT

SEQ ID NO: 27 - protein, 13 aa, Artificial Sequence
  source: /note=“Description of Artificial Sequence Synthetic peptide”
  source: /note=“N-term Ac”
  MOD_RES (10)..(10): 3-trifluoromethyl-3-phenyldiazarine
  source: /note=“C-term NH2”
DCAWHLGELXWCT

SEQ ID NO: 28 - protein, 13 aa, Artificial Sequence
  source: /note=“Description of Artificial Sequence Synthetic peptide”
  source: /note=“N-term Ac”
  MOD_RES (11)..(11): 3-trifluoromethyl-3-phenyldiazarine
  source: /note=“C-term NH2”
DCAWHLGELVXCT

SEQ ID NO: 29 - protein, 13 aa, Artificial Sequence
  source: /note=“Description of Artificial Sequence Synthetic peptide”
  source: /note=“N-term Ac”
  MOD_RES (13)..(13): 3-trifluoromethyl-3-phenyldiazarine
  source: /note=“C-term NH2”
DCAWHLGELVWCX

SEQ ID NO: 30 - protein, 23 aa, Unknown
  source: /note=“Description of Unknown tryptic peptide”
WQQGNVFSCSVMHEALHNHYTQK

SEQ ID NO: 31 - protein, 7 aa, Unknown
  source: /note=“Description of Unknown tryptic peptide”
DTLMISR

SEQ ID NO: 32 - protein, 16 aa, Homo sapiens
DTLMISRTPEVTCVVV

SEQ ID NO: 33 - protein, 16 aa, Homo sapiens
DTLYITREPEVTCVVV

SEQ ID NO: 34 - protein, 16 aa, Oryctolagus cuniculus
DTLMISRTPEVTCVVV

SEQ ID NO: 35 - protein, 16 aa, Mus sp.
DVLTITLTPKVTCVVV